(19) United States
(12) Patent Application Publication
(10) Pub. No.: US 2024/0108257 A1
(43) Pub. Date: Apr. 4, 2024

(54) WIRED IMPLANTABLE MULTIANALYTE MONOLITHIC INTEGRATED SENSOR CIRCUIT

(71) Applicant: Integrated Medical Sensors, Inc., Irvine, CA (US)

(72) Inventors: MUHAMMAD MUJEEB-U-RAHMAN, IRVINE, CA (US); MEISAM HONARVAR NAZARI, IRVINE, CA (US); MEHMET SENCAN, TUSTIN, CA (US)

(21) Appl. No.: 18/553,733

(22) PCT Filed: Mar. 8, 2023
(86) PCT No.: PCT/US23/14860

§ 371 Date: Oct. 3, 2023

Related U.S. Application Data
(60) Provisional application No. 63/318,790, filed on Mar. 11, 2022.
(60) Provisional application No. 63/333,443, filed on Apr. 21, 2022.
(60) Provisional application No. 63/336,299, filed on Apr. 28, 2022.
(60) Provisional application No. 63/346,739, filed on May 27, 2022.

(51) Int. Cl. A61B 5/1473; A61B 5/00

(52) U.S. Cl. CPC A61B 5/1473; A61B 5/0031; A61B 2562/028

(57) ABSTRACT

A multi-analyte sensor system is provided, comprising: a transmitter for containing a battery the transmitter being for placement on top of patient skin; and an implantable multianalyte monolithic integrated circuit for placement beneath the patient skin, wherein the implantable multianalyte monolithic integrated circuit comprises a sensor control circuit (e.g., Potentiostat, Voltametric Circuit) and an electrochemical sensing element, wherein the electrochemical sensing element is configured to sense concentrations of two or more analytes and generate an electrical signal representative of these concentrations, and wherein sensor control circuit is electrically connected to the electrochemical sensing element and enables independent control of detection method and conditions (e.g., redox potential) at the integrated sensing circuit.

### CROSS-REFERENCE TO RELATED APPLICATIONS

This application claims any and all benefits as provided by law, including benefits under U.S.C. 119(e) of U.S. Provisional Application No. 63/336,299, filed on Apr. 28, 2022 (Internal Reference: 003-USP4), U.S. Provisional Application No. 63/346,739, filed on May 27, 2022 (Internal Reference: 006-USP), U.S. Provisional Application No. 63/318,790, filed on Mar. 11, 2022 (Internal Reference: 009-USP), and U.S. Provisional Application No. 63/333,443, filed on Apr. 21, 2022 (Internal Reference: 012-USP), the contents of each of the above are incorporated herein by reference in their entirety.

### STATEMENT REGARDING FEDERALLY SPONSORED RESEARCH

This invention was made with government support under contract no. R44DK111001, and no. R43DK121621 awarded by the National Institutes of Health. The government has certain rights in the invention.

### BACKGROUND

### Field of the Invention

The present disclosure provides a scalable, flexible, electrochemical sensing platform for measuring multiple analytes, said platform including a transmitter, a wire, and a multianalyte monolithic integrated sensor circuit containing one or more integrated sensors.

### Description of the Related Art

Continuous monitoring of personal health has the potential to revolutionize healthcare by enabling preventative health management compared to traditional treatment strategies which rely upon a few measurements at discrete points. Continuous monitoring should enable the detection of diseases and conditions before users would be drawn to a doctor's office for a discrete blood test.

Realization of the potential of continuous monitoring of health requires new tools to measure analytes in the body effectively and continuously. There are currently some health monitoring devices being used to continuously measure in-vivo analyte levels. However, the technologies which have been deployed thus far have suffered from shortcomings preventing widespread adoption.

An excellent example of a continuous monitoring system that has come to market can be seen with Continuous Glucose Monitoring (CGM) systems. CGM systems from MEDTRONIC® and DEXCOM® are available for continuous glucose monitoring for diabetics using transdermal systems. Less than 10% of diabetics currently use Continuous Glucose Monitoring (CGM) systems of any kind, although it has been shown to be the best method for diabetes management.

Thus far, on market devices have been limited to detecting single analytes. For example, the CGM systems from MEDTRONIC® and DEXCOM® are limited to measuring glucose levels in the body.

However, in the exemplary case of diabetics, analytes beyond glucose are of extreme interest. A serious life-threatening complication of diabetes is diabetic ketoacidosis (DKA). When a person's cells do not get the glucose they need for energy, the body burns fat for energy which produces ketones. In DKA, ketones build up in the bloodstream turning blood more and more acidic. Accordingly, there exists a need for diabetics to know the level of ketones in their blood to prevent the development of DKA. Another life-threatening non-disease-specific complication of diabetes is lactate acidosis. One major treatment modality for type II diabetes is metformin. Metformin-associated acidosis has an estimated incidence of 4.3 per 100,000 person-years. Lactic acidosis is less frequent than some other complications with diabetes, but when present, the mortality rate may be as high as 50%. Thus, there exists a need for diabetics to know the level of lactate in their blood to prevent the development of lactate acidosis.

In view of these shortcomings, a need exists for a multianalyte continuous sensing system.

### SUMMARY

Accordingly, a clear need exists for an improved platform in the continuous health monitoring industry with the capability of measuring multiple analytes. The present disclosure addresses several of these shortcomings of the prior art to finally make continuous multianalyte health monitoring more mainstream.

In various instances, the present disclosure provides for a multianalyte monolithic integrated sensor circuit for placement inside the body which senses the concentration of an analyte by an electrode and transmits digital data to a transmitter outside the body. The multianalyte monolithic integrated sensor circuit and the transmitter are connected by a transdermal wire. By combining in a single multianalyte monolithic integrated circuit, the sensor chemistry driving analyte detection as well as the circuitry needed to translate the concentration of the analyte into digital information, embodiments of the present disclosure reduce complexity, size, and cost. Additionally, by placing the electronics adjacent to the electrodes which measure the analyte, the present disclosure permits the determination of a superior signal for a longer period as compared to traditional systems because of the permissive use of multiple discrete electrodes with minimal electronic signal travel. Moreover, this also reduces the complexity of the transmitter and hence enables a smaller and lighter transmitter. This increases sensor wear time and decreases the need of strong adhesives, hence minimizing skin irritation and allergies.

Unlike implantable wireless CGM systems, such as the EVERSENSE® system, the embodiments of the present disclosure present a wired connection for the transdermal delivery of power and, optionally, the exchange of digital information. The wired connection permits easier insertion of the device as well as a more stable supply of power. The transdermal wired connection also fixes the device allowing a smaller size. Lastly, the wire connection allows the device to be much more easily removed by the user.

According to a first aspect of the present disclosure, there is provided a monolithically integrated sensor chip configured for insertion into a patient comprising: at least two working electrodes for sensing two or more analytes and generating signals representative of analyte concentration, each working electrode paired with different respective potentiostats at independent working potentials, and a communication and power unit connected to the potentiostat, which power and transmits data representative of said analyte concentrations.

The monolithically integrated sensor chip may include independent working potentials are different working potentials.

The monolithically integrated sensor chip may include a potentiostat connected within half a millimeter to the entirety of at least one working electrode.

The monolithically integrated sensor chip may include a temperature sensor.

The monolithically integrated sensor chip may further comprise an digital to analog converter configured to set the independent working potentials of each respective at least two working electrodes.

The monolithically integrated sensor chip may further include at least one working electrode coated with a chemistry sensitive to one or a multitude of ketone bodies. Additionally, the monolithically integrated sensor chip may further include at least one working electrode coated with a chemistry sensitive to lactate. Additionally, the monolithically integrated sensor chip may further include at least one working electrode coated with a chemistry sensitive to glucose.

The monolithically integrated sensor chip may comprise an analog to digital converter, wherein the analog to digital converter is electrically connected to the potentiostat. The monolithically integrated sensor chip may be from 30 microns to 600 microns in thickness, 500 microns to 10,000 microns in length, and in a range from 100 microns to 4,000 microns in width.

The monolithically integrated sensor chip may have at least one working electrode patterned to increase surface area. The monolithically integrated sensor chip may have at least one working electrode patterned by forming holes.

The monolithically integrated sensor chip may have all potentiostats configured to be continuously powered by a battery.

The monolithically integrated sensor chip may have a communication or power unit configured to operate wirelessly.

The monolithically integrated sensor chip may have a communication or power unit configured to operate via a wired connection to a transmitter.

The monolithically integrated sensor chip may include a multiplexer electrically connected between the potentiostats associated with at least two working electrodes and the analog to digital converter.

The monolithically integrated sensor may further comprise a control logic programmed to process information from the at least two working electrodes by taking into account information from the temperature sensor.

In a second aspect of the present disclosure, there is provided an analyte monitoring system comprising the monolithically integrated sensor chip, a transcutaneous connector comprising at least one conductive path; and a transmitter for containing a battery, the transmitter for placement on top of patient skin.

The analyte monitoring system may be configured to be placed at a depth of 0.3 to 10 mm beneath the patient skin.

The analyte monitoring system may be a continuous analyte monitoring system.

According to a third aspect of the present disclosure, there is provided a monolithic integrated sensor chip configured for insertion into a patient comprising an integrated sensing and data acquisition unit for sensing two or more analytes and generating signals representative of analyte concentration, said integrated sensing and data acquisition unit having at least two working electrodes, a first working electrode configured to work via a potentiostat and a second working electrode configured to work via voltammetry; and an integrated communication and power unit connected to the integrated sensing unit, which powers the integrated sensing and data acquisition unit and transmits data representative of said analyte concentrations.

The monolithically integrated sensor chip may have independent working potentials are different working potentials.

The monolithically integrated sensor chip may have a potentiostat connected within half a millimeter to the entirety of the first working electrode.

The monolithically integrated sensor chip may further comprise a temperature sensor.

The monolithically integrated sensor chip may further comprise a digital to analog converter configured to set the independent working potentials of each respective at least two working electrodes.

The monolithically integrated sensor chip may have at least one working electrode coated with a chemistry sensitive to one or more ketone bodies. The monolithically integrated sensor chip may have at least one working electrode coated with a chemistry sensitive to lactate. The monolithically integrated sensor chip may have at least one working electrode coated with a chemistry sensitive to glucose.

The monolithically integrated sensor chip may further comprise an analog to digital converter, wherein the analog to digital converter is electrically connected to the potentiostat.

The monolithically integrated sensor chip may be from 30 microns to 600 microns in thickness, 500 microns to 10,000 microns in length, and in a range from 100 microns to 4,000 microns in width.

The monolithically integrated sensor chip may have at least one working electrode patterned to increase surface area. The monolithically integrated sensor chip may have at least one working electrode patterned by forming holes.

The monolithically integrated sensor chip may have all potentiostats configured to be continuously powered by a battery.

The monolithically integrated sensor chip may have a communication and power unit configured to be operated wirelessly.

The monolithically integrated sensor chip may have a communication and powered unit configured to be operated via a wired connection to a transmitter.

The monolithically integrated sensor chip may further comprise a multiplexer electrically connected between the potentiostats associated with at least two working electrodes and the analog to digital converter.

The monolithically integrated sensor chip may further comprise a control logic programmed to process information from the at least two working electrodes by taking into account information from the temperature sensor.

According to a fourth aspect of the present disclosure, there is provided a continuous sensor assembly comprising a flexible substrate and a monolithically integrated sensor chip comprising at least two working electrodes and surrounded on all sides by the flexible substrate except for wherein a top portion of the monolithically sensor chip is open to an analyte.

The continuous sensor assembly may have any monolithically integrated sensor chip described above.

The continuous sensor assembly may have a flexible substrate with a rigidity enforcing member. The continuous sensor assembly may have a rigidity enforcing member of metal and a thickness of 5 to 60 microns. The thickness of the rigidity enforcing members may be about 30 microns.

According to a fifth aspect of the present disclosure, there is provided a method of packaging a monolithically integrated sensor chip, said method comprising bonding a front of the monolithically integrated sensor chip to a conductor located on a flexible substrate, and covering the entirety of a back of the monolithically integrated sensor chip with a flexible material, wherein the flexible substrate and the flexible material cover all of the sides and back of the monolithically integrated sensor chip and only a portion of the front of the monolithically integrated sensor chip.

The method of packaging a monolithically integrated sensor chip may have any monolithically integrated sensor chip described above.

According to a sixth aspect of the present disclosure, there is provided a method of post-processing a monolithically integrated sensor chip, said method comprising lithographic patterning, metal etching, physical vapor deposition, and dicing such that parts of the CMOS sensor chip are coated with two different suitable material including two different noble metals while the remaining of the integrated sensor chip is not coated with either material.

The method of post-processing a monolithically integrated sensor chip with the post-processing done at wafer scale.

According to a seventh aspect of the present disclosure, there is provided a method of functionalizing a monolithically integrated sensor chip comprising at least two noble metal coated working electrodes, said method comprising coating a portion of the monolithically integrated sensor chip with a water-dissolvable sacrificial layer; coating a portion of the monolithically integrated sensor chip with a mask; patterning the mask to open the mask over a first noble metal coated working electrode; transfer to the sacrificial layer by exposure to water creating an opening over the first noble metal coated working electrode; coating the first working electrode(s) with a first hydrogel; and coating the wafer in an excess of water.

The method of functionalizing a monolithically integrated sensor chip may further comprise coating a portion of the monolithically integrated sensor chip with a water-dissolvable sacrificial layer; coating a portion of the monolithically integrated sensor chip with a mask; patterning the mask to open the mask over a second noble metal coated working electrode; transfer to the sacrificial layer by exposure to water creating an opening over the second noble metal coated working electrode; coating the second working electrode(s) with a second hydrogel different from the first hydrogel; and coating the wafer in an excess of water to form a monolithically integrated sensor chip with two working electrodes with two different coated hydrogels.

The method of functionalizing a monolithically integrated sensor chip sacrificial layer may be water soluble polyacrylic acid (PAA).

The method of functionalizing a monolithically integrated sensor chip mask may be a photo pattern able material.

According to an eighth aspect of the present disclosure, there is provided a method of attaching a monolithically integrated sensor configured to measure at least two analytes to a flexible electronics connector for placement within a human subject, said method comprising: attaching the monolithically integrated sensor configured to measure at least two analytes to the electronic connector by placing a conductor between a contact pad of the monolithically integrated sensor and a contact pad of the electronics connector, wherein the overall thickness of the integrated sensor and flexible electronics connector after attachment is less than 1 mm and protects the contact pads to avoid contact with human fluids but provides for access of human fluids to integrated sensing elements of the integrated sensor permitting measurement of the at least two analytes.

The method of attaching a monolithically integrated sensor may have an overall thickness of the integrated sensor and flexible electronics connector after attachment is less than 150 microns.

According to a ninth aspect of the present disclosure, there is provided a method of adding at least two functional chemistry layers sensitive to at least 2 different analytes on a monolithically integrated sensor on a CMOS wafer to selectively coat the sensing electrodes, said method comprising coating a first functional chemistry layer on sensing electrode(s) sensitive to a first analyte on the CMOS wafter by pico droplet deposition, spin coating, lithographic patterning, spray coating, stencil coating, or dip coating, and coating a second functional chemistry layer on sensing electrode(s) sensitive to a second analyte on the CMOS wafter by pico droplet deposition, spin coating, lithographic patterning, spray coating, stencil coating, or dip coating.

The method of adding at least two functional chemistry layers may have a first functional chemistry layer coated at a thickness of less than 100 um. The first functional chemistry layer may be coated at thickness of less than 10 um.

According to a tenth aspect of the present disclosure, there is provided a transmitter system for placement on patient skin comprising an integrated circuit and a power source both located on a flexible electronics substrate and used to power and communicate with a subdermal sensor configured to measure one or more analytes, wherein the transmitter is smaller than 3 cm in the largest dimension and weighs less than 20 grams.

The transmitter system may have the transmitter encased in a thin housing with a thickness of less than 5 mm that protects it from the environment without the use of hard casing.

According to an eleventh aspect of the present disclosure, there is provided a transmitter system for placement on patient skin comprising an integrated circuit and a power source both located on a rigid-flexible printed circuit board including a rigid substrate and a flexible substrate, said integrated circuit and power source used to power and communicate with a subdermal sensor configured to measure one or more analytes, wherein the rigid substrate is smaller than 3 cm in the largest dimension and weighs less than 20 grams, wherein the sensor is connected to the flexible substrate, and wherein the rigid and the flexible substrates are connected with conductive vias without the use of an interfacial connector.

The transmitter system may have the transmitter encased in a thin housing with a thickness of less than 5 mm that protects it from the environment without the use of hard casing.

### BRIEF DESCRIPTION OF THE FIGURES

Embodiments of the present invention will now be described, by way of example, with reference to the accompanying drawings, in which:

FIG. 1 is an image of an implantable multianalyte monolithic integrated circuit in accordance with one embodiment.

FIG. 2 is a diagrammatic view of a continuous diabetic multianalyte monitoring system in accordance with one embodiment.

FIG. 3A is a top schematic view of an implantable multianalyte monolithic integrated circuit in accordance with one embodiment.

FIG. 3B is a side cross-sectional schematic view of an implantable multianalyte monolithic integrated circuit in accordance with one embodiment.

FIG. 4 is a flow diagram of an exemplary process for fabricating a continuous multianalyte monitoring system in accordance with one embodiment.

FIG. 5 is a circuit block diagram of an embodiment of an implantable multianalyte monolithic integrated circuit and the corresponding transmitter in accordance with one embodiment.

FIG. 6 is a circuit block diagram of an embodiment of an implantable multianalyte monolithic integrated circuit in accordance with one embodiment.

FIG. 7 is a circuit block diagram of an embodiment of an implantable multianalyte monolithic integrated circuit in accordance with one embodiment.

FIG. 8 is a circuit of an element of a temperature sensor of an implantable multianalyte monolithic integrated circuit in accordance with one embodiment.

FIG. 9 is a circuit of a temperature sensor of an implantable multianalyte monolithic integrated circuit in accordance with one embodiment.

FIG. 10 is a graph of current output of a temperature sensor of an implantable multi analyte monolithic integrated circuit in accordance with one embodiment.

FIG. 11 is a circuit schematic of a potentiometric (voltammetric sensing) sensor of an implantable multianalyte monolithic integrated circuit in accordance with one embodiment.

FIG. 12 is a circuit schematic of a potentiostat (amperometric sensing) of an implantable multianalyte monolithic integrated circuit in accordance with one embodiment.

FIG. 13 is a circuit of an analog-to-digital converter (ADC) of an implantable multianalyte monolithic integrated circuit in accordance with one embodiment.

FIG. 14 is a circuit diagram of a digital-to-analog converter (DAC) of an implantable multi analyte monolithic integrated circuit in accordance with one embodiment.

FIG. 15 is a circuit schematic for sensing using time multiplexing of an implantable multianalyte monolithic integrated circuit in accordance with one embodiment.

FIG. 16 is a circuit schematic for sensing using time multiplexing of an implantable multianalyte monolithic integrated circuit in accordance with one embodiment in which the amperometric and voltametric measurements can be done using the same device.

FIG. 17 is a top schematic view of a series of implantable multianalyte monolithic integrated circuits in accordance with an embodiment.

FIG. 18 is a schematic view of different embodiments of the connector (between the sensing platform and the transmitter) of the continuous multi-analyte monitoring system.

FIG. 19 is a flow diagram of an exemplary process for fabricating a continuous multianalyte monitoring system in accordance with one embodiment.

FIG. 20 is a cross-sectional view of two different embodiments of CMOS sensing chip as received from a CMOS foundry according to an embodiment.

FIG. 21 is a cross-sectional flow diagram of post-processing of top metal layers of a CMOS stack of an implantable multianalyte monolithic integrated circuit in accordance with one embodiment.

FIG. 22 is a cross-sectional flow diagram of post-processing of top metal layers of a CMOS stack of an implantable multianalyte monolithic integrated circuit wherein different on-chip electrodes are coated with a different surface material in accordance with one embodiment.

FIG. 23 is a cross-sectional flow diagram of functionalization of a CMOS stack of an implantable multianalyte monolithic integrated circuit in accordance with one embodiment using droplet dispensing method

FIG. 24 is a cross-sectional view of functionalized implantable multianalyte monolithic integrated circuits using droplet deposition and using, for example, rocking or spinning to achieve uniform surface coatings within the sensing wells.

FIG. 25 is a cross-sectional flow diagram of functionalization of a CMOS stack of an implantable multianalyte monolithic integrated circuit using a sacrificial layer patterning method in accordance with one embodiment.

FIG. 26 is a cross-sectional flow diagram of surface functionalization of a CMOS stack of an implantable multianalyte monolithic integrated circuit with a thin membrane coating followed by a hydrogel coating in accordance with one embodiment.

FIG. 27 is a cross-sectional flow diagram of post-processing functionalization of a CMOS stack of an implantable multi analyte monolithic integrated circuit for coating 3 enzyme layers on a single device (expansion on 2-layer process shown in FIG. 25) in accordance with one embodiment.

FIG. 28 is a cross-sectional view of a CMOS stack of an implantable multianalyte monolithic integrated circuit after surface functionalization and with and without a protective layer in accordance with one embodiment.

FIG. 29 is a cross-sectional view of a step 1 and a step 2 of a packaging process of a connector and implantable multianalyte monolithic integrated circuit in accordance with one embodiment.

FIG. 30 is a cross-sectional view of a step 3 and a step 4 of a packaging process of a connector and implantable multianalyte monolithic integrated circuit in accordance with one embodiment.

FIG. 31 is a cross-sectional view of a step 5 and a step 6 of a packaging process of a connector and implantable multianalyte monolithic integrated circuit in accordance with one embodiment.

FIG. 32 is a cross-sectional view of a step 7 of a packaging process of a connector and implantable multianalyte monolithic integrated circuit in accordance with one embodiment.

FIG. 33 is a top view flow diagram of functionalization of a packaged connector and implantable multianalyte monolithic integrated circuit using sacrificial layers in accordance with one embodiment.

FIG. 34 is a top view flow diagram of functionalization of a packaged connector and implantable multianalyte monolithic integrated circuit using droplet deposition and spray coating in accordance with one embodiment.

FIG. 35 is a top view flow diagram of functionalization of a packaged flexible connector and implantable multianalyte monolithic integrated circuit using photosensitive hydrogels in accordance with one embodiment.

FIG. 36 is a cross-section view of a pre-functionalized and a functionalized implantable multianalyte monolithic integrated circuit bonded with a flexible connector in accordance with one embodiment.

FIG. 37 is cross-sectional view of a continuous multianalyte monitoring system having a transmitter consisting of a circuit board and electronics components in accordance with one embodiment.

FIG. 38 is an image of a continuous multianalyte monitoring system showing the packaging and internal components of the transmitter in accordance with one embodiment.

FIG. 39 is cross-sectional view of a continuous multianalyte monitoring system that uses a system on chip (SoC) together with a miniature battery in accordance with one embodiment.

FIG. 40 is circuit block diagram of a transmitter in accordance with one embodiment.

FIG. 41 is circuit block diagram of a transmitter that uses a capacitor and a resistor to couple and de-couple the power and data in accordance with one embodiment.

FIG. 42 is cross-sectional view of a continuous multianalyte monitoring system which uses a rigid-flex PCB in accordance with one embodiment.

FIG. 43 is cross-sectional view of a continuous multianalyte monitoring system in which the transmitter uses a flexible electronics substrate and a connector to connect with the sensor in accordance with one embodiment.

FIG. 44 is cross-sectional view of a continuous multianalyte monitoring system in which the transmitter uses a flexible electronics substrate without requiring a connector to connect with the sensor in accordance with one embodiment.

FIG. 45 is cross-sectional view of a continuous multianalyte monitoring system in which the transmitter is implemented on a flexible substrate and uses thin insulating coatings in accordance with one embodiment.

FIG. 46 is an algorithmic scheme to program the transmitter of a continuous multianalyte monitoring system in accordance with one embodiment.

FIG. 47 is a graph of peroxide response on an implantable multianalyte monolithic integrated circuit in accordance with one embodiment.

FIG. 48 is a graph of glucose response on an implantable multianalyte monolithic integrated circuit in varying glucose concentrations in accordance with one embodiment.

FIG. 49 is an in vitro graph of glucose and peroxide response on an implantable multianalyte monolithic integrated circuit in accordance with one embodiment.

FIG. 50 is a graph of glucose and ketone response on an implantable multianalyte monolithic integrated circuit in a solution of varying glucose concentration in accordance with one embodiment.

FIG. 51 is a graph of glucose and ketone response on an implantable multianalyte monolithic integrated circuit in a solution of varying ketones concentration in accordance with one embodiment.

FIG. 52 is a top view of an implantable multianalyte monolithic integrated circuit coated with a Glucose sensing (Gox) and a Ketone sensing (KDH) layer in accordance with one embodiment.

FIG. 53 is a graph of glucose and lactate response on an implantable multianalyte monolithic integrated circuit in a solution of varying glucose concentration in accordance with one embodiment.

FIG. 54 is a graph of glucose and lactate response from a single implantable multianalyte monolithic integrated circuit in a solution of varying lactate concentration in accordance with one embodiment.

FIG. 55 is a top view of an implantable multianalyte monolithic integrated circuit coated with a Glucose Oxidase (Gox) and a Lactate Oxidase (Lox) layer in accordance with one embodiment.

FIG. 56 is a graph of glucose and peroxide response on an implantable multianalyte monolithic integrated circuit tested in a human in accordance with one embodiment.

FIG. 57 is a graph of current in response to temperature fluctuation from the temperature sensor of an implantable multianalyte monolithic integrated circuit in accordance with one embodiment.

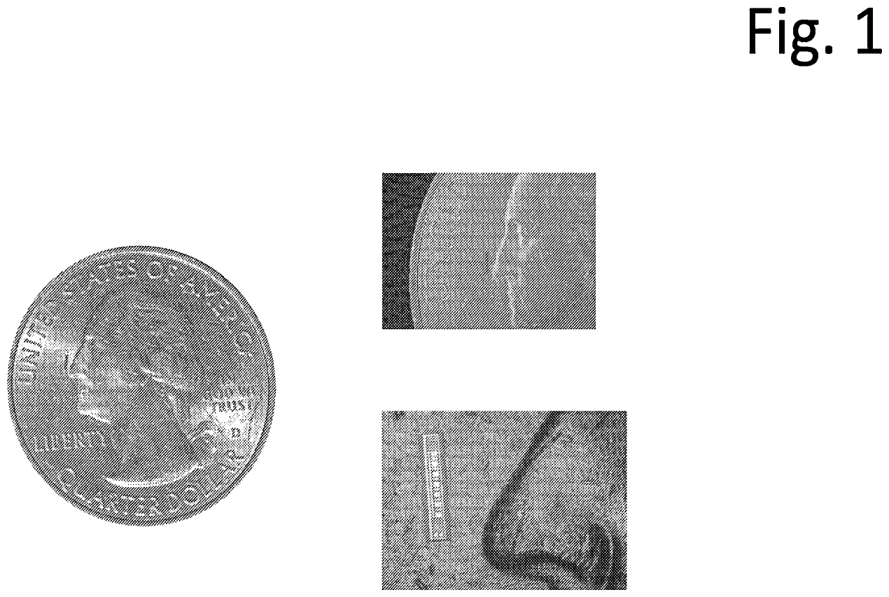

### DETAILED DESCRIPTION

The present disclosure is directed to an implantable multianalyte monolithic integrated sensor circuit and system that can be used in a variety of in-vivo applications providing continuous measurement of multiple types of health or biological markers (e.g., metabolites). Glucose, ketones, and lactate are example analytes discussed herein. As a person having ordinary skill in the art will appreciate, the described devices, systems and methods can be more generally applied to other analytes and analyte combinations. Additionally, some ex-vivo uses are easily envisioned.

### Definitions

To facilitate an understanding of the preferred embodiment, a number of terms are defined below.

The term “analyte” as used herein is a broad term, and is to be given its ordinary and customary meaning to a person of ordinary skill in the art (and is not to be limited to a special or customized meaning), and refers without limitation to a substance or chemical constituent in a fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the sensing regions, devices, and methods is glucose. Salts, sugar, protein, fat, vitamins, and hormones naturally occurring in blood or interstitial fluids can also constitute analytes in certain embodiments. The analyte can be naturally present in the fluid, for example, a metabolic product, a hormone, an antigen, an antibody, and the like. Alternatively, the analyte can be introduced into the body, for example, a contrast agent for imaging, a radioisotope, a chemical agent, a fluorocarbon based synthetic blood, or a drug or pharmaceutical. The metabolic products of drugs and pharmaceutical compositions are also contemplated analytes.

The phrase “continuous analyte sensing” as used herein is a broad term and is to be given its ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every 5 seconds or less to about one minute or more, preferably from about 10, 15, 20, 25, 30, 35, 40, 45, 50, 55, or 60 seconds.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are acetaminophen and ascorbic acid.

The term “sensing membrane” as used herein is a broad term, and is to be given its ordinary and customary meaning to a person of ordinary skill in the art (and is not to be limited to a special or customized meaning), and refers without limitation to a permeable or semi-permeable membrane that can comprise one or more domains and that is constructed of materials having a thickness of a few microns or more, and that are permeable to reactants and/or co-reactants employed in determining the analyte of interest. As an example, a sensing membrane can comprise polyurethane.

A “functionalization layer” as used herein is a broad term, and is to be given its ordinary and customary meaning to a person of ordinary skill in the art (and is not to be limited to a special or customized meaning), and refers without limitation to a hydrogel that can comprise one or more enzymes and that is constructed to be of a thickness of a few microns or more. As an example, a functionalization layer can comprise a BSA hydrogel with glucose oxidase contained therein.

A “monolithic substrate” is a substrate, upon which components are monolithically integrated and therefore such components are not adhered to and/or secured via mechanical means to the substrate. In various embodiments according to the present disclosure the monolithic substrate can be the result of processing using CMOS technology or other fabrication technology known to the skilled person. It is understood that a monolithic substrate has multiple faces, and at least a first face and a second face. A first and second face can be distinguished from other faces of the monolithic substrate in that the first and second face are larger than the other faces of the monolithic substrate.

The term “sensing element” refers to the region of the device responsible for the detection of a particular biological indicator. For example, in some embodiments for glucose monitoring, the sensing element refers to that region wherein a biological sample (e.g., blood or interstitial fluid) or portions thereof contacts an enzyme (e.g., glucose oxidase); a reaction of the biological sample (or portion thereof) results in the formation of reaction products that allow a determination of the glucose level in the biological sample.

The term “power source” is intended to have its ordinary meaning in the art. In various embodiments according to the present disclosure the power supply can comprise an RF antenna or photovoltaic cell for receiving external energy.

### System Design

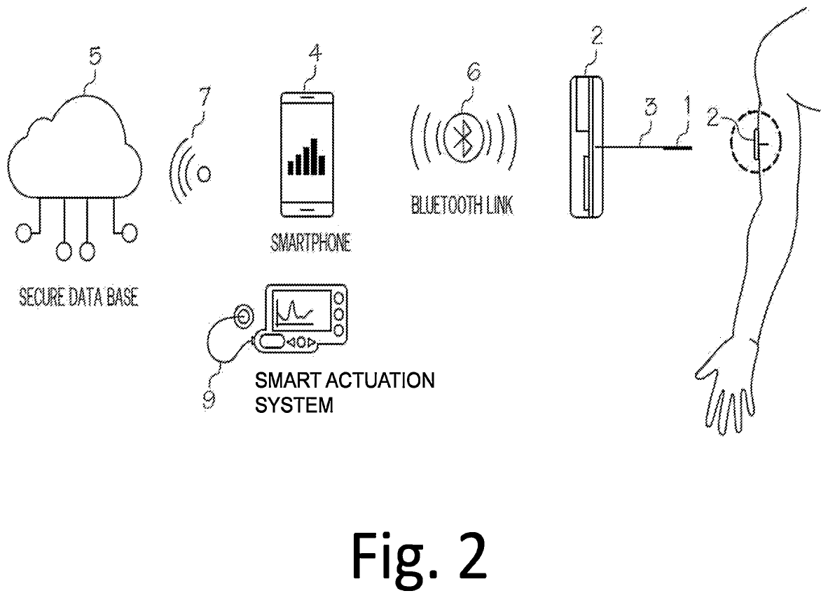

An overview of an embodiment of the system of the present disclosure can be seen in FIG. 2. The system can include a multianalyte monolithic integrated sensor circuit 1 that is connected to an external transmitter 2 through a connector 3 made of conductive material which may be coated with a biocompatible polymer. The connector 3 may be a flexible connector. The transmitter 2 provides electrical power to the multianalyte monolithic integrated circuit 1 via the flexible connector 3. It also communicates to the multi analyte monolithic integrated circuit 1 via the flexible connector 3. The communication can be bi-directional or unidirectional. Transmitter 2 can be wirelessly linked to a smart reader (e.g., smartphone) 4, smart actuation device (e.g., an insulin pump/pen) 9, and/or a secure database 5 using a wireless communication technology such as Bluetooth, Zigbee, or WiFi 6, 7. Alternatively, the smart reader 4 can act as a bridge between the transmitter and the secure database 5. The details of the important parts of the system are provided next.

### Multianalyte Monolithic Integrated Sensor Design

A) Overview

In accordance with some embodiments of the disclosure, the multianalyte monolithic integrated sensing circuit is, for example, an integrated circuit chip fabricated using CMOS fabrication technologies known to the person skilled in the art.

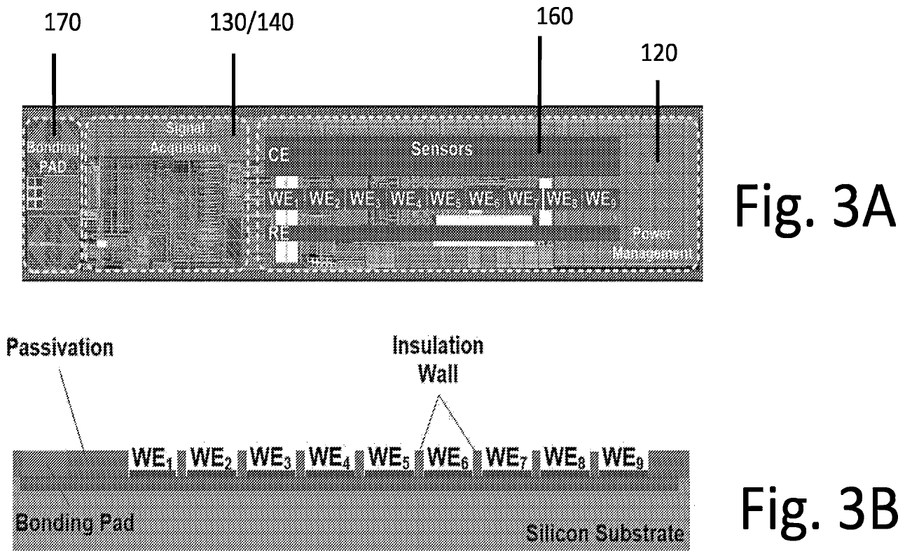

An overview of an embodiment of the multianalyte monolithic integrated sensor circuit 1 of the present disclosure can be seen in FIG. 3. FIG. 3A shows a top schematic view.

As seen in FIG. 3A, the multianalyte monolithic integrated sensor circuit 1 can include an integrated sensing element 160. The integrated sensing element 160 may also be referred to as an “electrochemical sensing element”. The integrated sensing element can comprise counter electrodes, working electrodes, and reference electrodes. The integrated sensing element contains at least two working electrodes. The multianalyte monolithic integrated circuit can further contain a power management unit 120, a sensor signal acquisition unit 130, a communication unit 140, and an optional interface unit 175. The interface unit in one embodiment consist of one or more contact pads 170. The interface unit may provide further circuitry to better communicate with other components of the system, e.g., the transmitter. The interface unit is most often wired in the present instance. In its simplest embodiment, the interface unit may consist of a couple of contact pads that provide a convenient interface for the wired connector which links the transmitter. The multianalyte monolithic integrated sensor circuit includes on-chip connectors in the interface unit. Exemplary dimensions are length 1 mm and width 0.15 mm and a thickness of 50 μm.

FIG. 3B shows a cross-sectional view of one embodiment of the multianalyte monolithic integrated sensor circuit. The integrated sensing element can comprise insulation wall(s) separating distinct working electrodes. The integrated sensing element can comprise passivation in areas not post-processed and/or functionalized. In a packaged form, on-chip connectors can contact a bonding bump and thus electrically connect to a flexible board connector. The CMOS stack of the multianalyte monolithic integrated sensor circuit 1 is formed from/on a silicon substrate (e.g., silicon wafer).

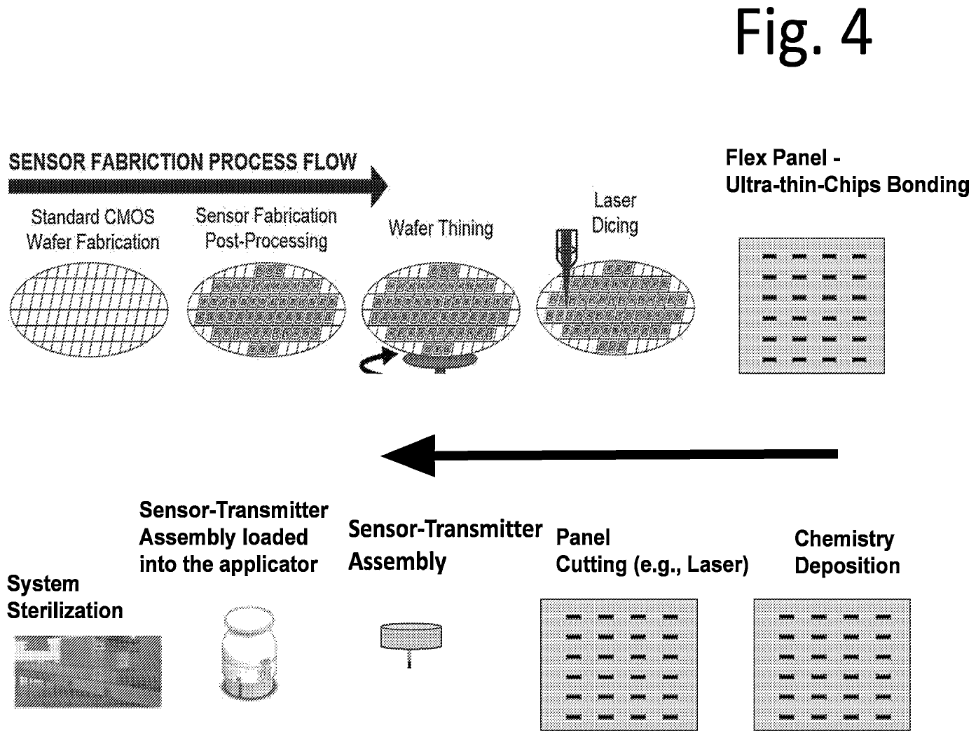

An embodiment of fabricating the integrated sensor 1, the flexible connector 3, the transmitter 2, and the applicator (inserter) 8 and assembling them together in a scalable manner is in FIG. 4. This fabrication uses a series of process steps commonly used in the semiconductor industry including CMOS fabrication, postprocessing, wafer thinning, dicing, bonding of the dies to flex panel, chemistry (enzyme, PU chemistry) deposition, panel cutting, assembly of the sensor with the transmitter and the applicator, and sterilization.

An important element of the multianalyte monolithic integrated circuit is the small size of the chip. The multianalyte monolithic integrated sensor circuit can include many interconnected functional modules or subsystems and can be in a range from 30 microns to 600 microns in thickness (e.g., 50 microns to 150 microns), 500 microns to 10,000 microns in length (e.g., 1500 microns to 3000 microns) and in a range from 100 microns to 4,000 microns in width (e.g., 400 microns to 1000 microns).

The small size of the multianalyte monolithic integrated sensor circuit along with shaping can minimize scar tissue formation in the body to a point where it only helps in keeping the system position stable but does not significantly isolate the implantable multianalyte monolithic integrated sensor circuit from accessing body fluids. This allows real-time measurement of important analytes (e.g., metabolic glucose level) for critical applications requiring instant changes to be reported as soon as possible (e.g., for hypoglycemic diabetic patients).

Designing the multianalyte monolithic integrated circuit in accordance with the specific implantation/insertion site (tissue orientation etc.) can help in reducing post-implantation complexities. For example, for implantation/insertion in biological tissues, the sensing platform can be shaped to minimize sharp edges to minimize tissue damage and hence immune system response. The multianalyte monolithic sensor circuit can be shaped to be longer in one dimension and much smaller in other dimensions to inject or insert the monolithic circuit using very small needles. This also allows the monolithic circuit to fit within the subcutaneous or subdermal space more easily. Minimizing the device thickness and coating it with a biocompatible soft material can also make it more flexible and reduce tissue damage.

A precisely controlled minimization of solid-state sensor size also reduces detection noise levels and can increase the Signal-to-Noise ratio (SNR), thus improving the sensitivity of the sensor. This miniaturization and accompanying SNR improvement is not possible without the added on-chip circuitry which can read the low current without much added noise of a longer-distance transmission. Furthermore, a compact integrated design minimizes contact resistance and capacitance between the sensor and the electronics, further enhancing sensitivity by improving the SNR of the sensor. Moreover, the decrease in electrode size reduces its capacitance which further reduces non-faradaic (charging) currents, thus improving SNR and decreasing the time it takes for the sensor to stabilize. The minimum SNR for a reliable detection is typically considered to be 3 (Signal to Noise Ratio: unitless).

In glucose oxidase functionalized embodiments, when in patient, the integrated sensor circuit of the present disclosure is capable of SNR ranges of 5-30, or more preferably 10-20. For lactate, the typical SNR range is 5-20 and for Ketones the typical SNR range is 5-15. The SNR is higher in the case of glucose due to the availability of a more sensitive chemistry. In post-processed form, when in peroxide, the integrated sensor circuit of the present disclosure is capable of SNR ranges of 5-100, or preferably 60-100, or more preferably 70-80. (Please see, Donald M. Morgan and Stephen G. Weber, Noise and Signal-to-Noise Ratio in Electrochemical Detectors, Anal. Chem. 1984, 56, 13, 2560-2567, herein incorporated by reference in its entirety).

Sensor fabrication can start with submitting the chip design files to a semiconductor manufacturer (e.g., TSMC (Taiwan), ON Semiconductor (Phoenix, AZ)). The standard semiconductor fabrication processes can generate standard wafers of certain sizes (e.g., 12-inch diameter wafers). To reduce the dimension of the device, the original thickness (e.g., 750 μm) of the semiconductor wafer can be thinned down (e.g., to 50-250 μm) through mechanical grinding, chemical and/or mechanical polishing or chemical etching (e.g., Xenon Difluoride (XeF₂) etching from backside). This step can be done before or after surface functionalization and membrane chemistry deposition. Once thinned, the silicon becomes more flexible and can improve the integration of the sensor device within the surrounding tissue and reduce foreign body response. Thinning and/or grinding can be performed by a thinning and grinding facility (e.g., Advanced International Technologies, Quick-Pak). Some common exemplary CMOS process nodes which can be used for fabrication of the multianalyte monolithic integrated sensor are TSMC 180 nm, 65 nm, 55 nm, 250 nm, 90 nm.

Different types of dicing methods (saw, laser, stealth, etc.) along with some polishing methods can be used to realize any desirable shape (e.g., circular, rectangular, oval). Laser cutting can be used to form rounded edges on the final diced device and help reduce potential implantation injury and subsequent foreign body response. Laser dicing can be accompanied by appropriate environmental condition (e.g., oxygen flow) to create a thin layer of thermal oxide on sidewalls during dicing. Steam can also be used to generate a wet oxide on sensor sidewalls. Sidewall polishing after dicing can also be used to reduce and remove sharp edges. Further, coating with biocompatible membranes can also be used to minimize any sharp edges.

B) Core Circuit Block Diagram

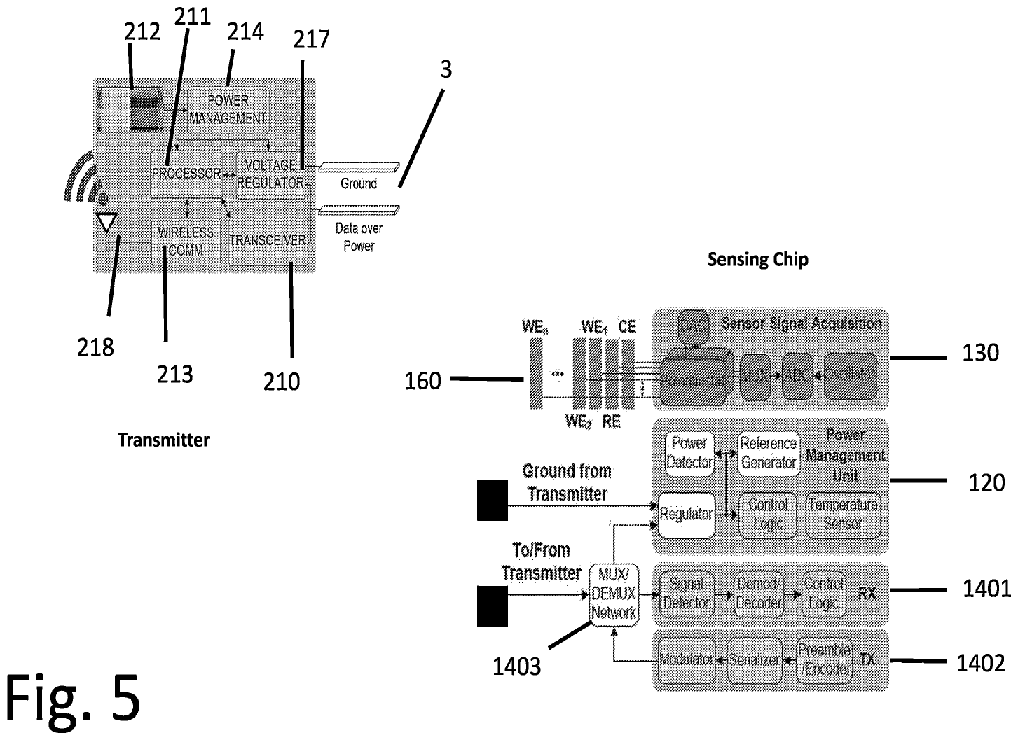

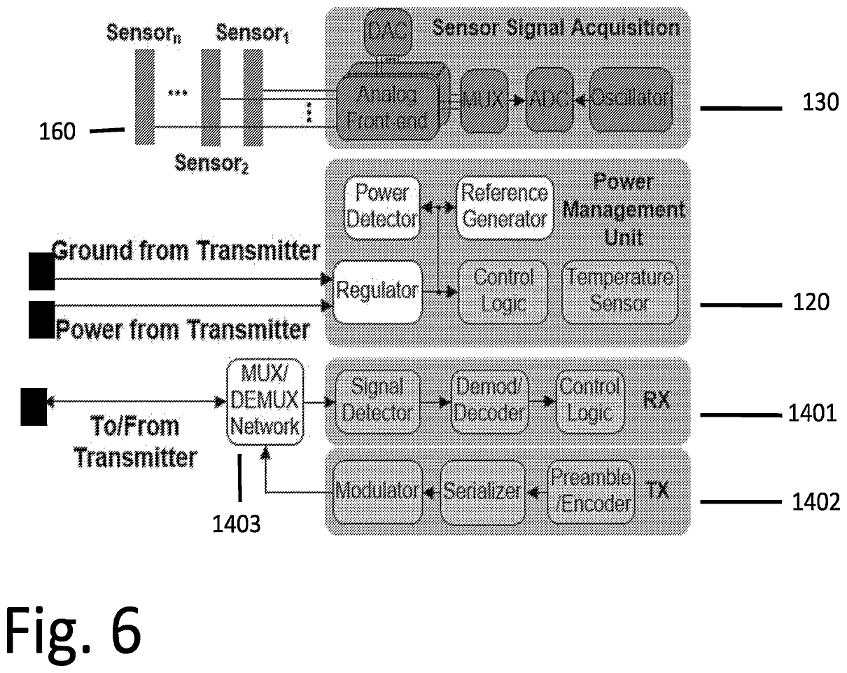

A more specific design view of an embodiment can be seen in FIG. 5 and FIG. 6.

The multianalyte monolithic integrated circuit again includes an integrated sensing element 160, sensor signal acquisition unit 130, power management unit 120, and a communication unit 140. The communication unit 140 further comprises a receiver subsystem (RX) 1401, a transmitter subsystem (TX) 1402, and a MUX/DEMUX network 1403 to separate the communication signal for the power signal.

FIG. 5 shows an interface unit comprising two electrical pads. One pad functions as ground from the transmitter. The other pad functions as data over power connected to the MUX/DEMUX network 1403. FIG. 5 contains a specific example wherein data is separated from power.

In a separate embodiment shown in FIG. 6, distinct conductors provide ground, power, and data from the transmitter 2 to implantable multianalyte monolithic integrated sensor circuit 1. In this instance, the interface unit comprises three pads. A pad is connected to ground into the power management unit 120. A pad is connected to power into the power management unit 120. A pad functions as data connected to the MUX/DEMUX network 1403.

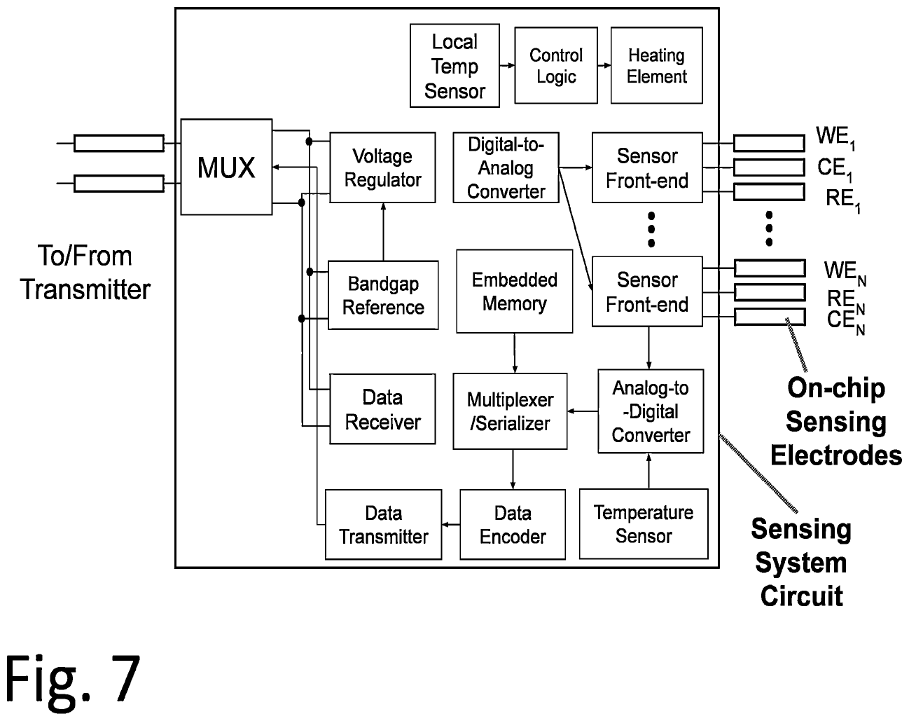

Another embodiment is shown in FIG. 7. As shown in FIG. 7, the monolithic integrated sensor circuit can include N on-chip sensors (each consisting of a set of on-chip sensing electrodes) with respective front-ends, an analog to digital converter, a temperature sensor, multiplexer/serializer, embedded memory, data encoder, data transmitter, data receiver, clock recovery, rectifier, voltage regulator, bandgap reference. The voltage regulator, the MUX (Multiplexer/Demultiplexer network) data receiver, data transmitter, data encoder, multiplexer/serializer, and embedded memory can function together to receive data from the external transceiver, receiver power from the external transceiver, as well as transmit data to the external transceiver (transmitter). In some cases, an on-chip heating element can also be used to cause a change in temperature locally to test chip functionality as well as help with a chip function (e.g., to clean the sensor surface).

As shown in FIG. 5 and FIG. 6, the receiver subsystem 1401 can compose of a signal detector, a demodulator/detector, and control logic. In the receiver subsystem 1401, the signal detector (e.g., envelope detector) can be used to extract the data transmitted from the external transmitter through, for example, a two-wire flexible connector.

A data encoding scheme (such as pulse width modulation or coding, pulse interval modulation or coding, pulse code modulation or coding, pulse count modulation or coding, Manchester Coding etc.) is used to send data from the transmitter to the sensor. Pulse code modulation being a preferred embodiment. Therefore, a data demodulator/decoder is utilized in the sensor receiver to decode the received signal which can include an activation tag for the implant as well as the sensor current measurement range. A control logic can perform signal conditioning and interpretation of the received data from the external transmitter 2.

As shown in FIG. 5 or FIG. 6, the transmission subsystem 1402 can include a preamble/encoder, serializer, and modulator. The TX unit can take sensor data from the sensor signal acquisition unit (e.g., ADC), encode it using a specific data encoding scheme for minimizing communication error (e.g., Pulse count coding), add predefined sequences (e.g., preambles, pilot sequences) and transmit the encoded data to the external transmitter using data communication-over-power scheme.

In the transmission subsystem 1402, the preamble/encoder can combine the sensor data into one or more packets that can be sent to the external transmitter. The packetized data can include the sensors (e.g., electrochemical, temperature) measured data and power level indicator. For example, the preamble/encoder can, in embodiments, combine all the data elements into a single data packet and add a preamble sequence at the beginning of the data packet for ease of detection by the external transmitter 2. The serializer can serialize data packets received from the preamble/encoder. An error detecting and/or correcting sequence (e.g., cyclic redundancy check or CRC, hamming code) can be added to the packets for immunity to communication and detection noise. The modulator can take the data in digital form and change it into waveform for sending to the transmitter 2 over the wire 3 of the implantable sensor circuit.

As shown in FIG. 5 or FIG. 6, the power management unit 120 can include a regulator (e.g., low V-LDO regulator), reference generator, power detector, temperature sensor, and control logic. In some embodiments the power management may also include a voltage limiter. The regulator can be a low-dropout regulator that regulates the incoming supply signal from the transmitter through a two-wire connector (which is an example of the wire 3) into a clean DC voltage (without ripples).

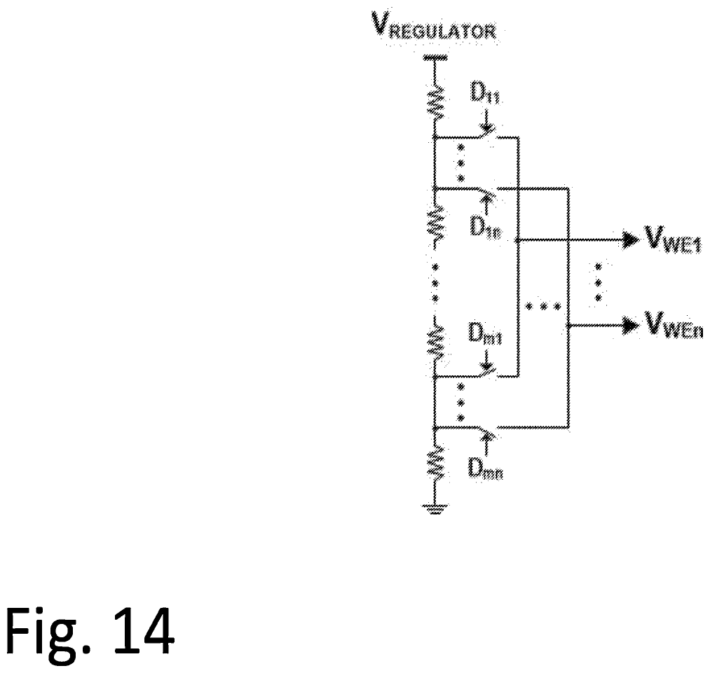

The reference generator can generate the reference voltages and currents used by the ADC, potentiostats, voltametric sensing circuits, and the oscillator of the sensor signal acquisition unit 130. The reference generator can provide high power supply rejection to eliminate sensitivity to supply ripple. Although below the sensor signal acquisition unit 130 is described as optionally containing the digital to analog converter (DAC) of FIG. 14 to allow the generated reference voltages to be programmable, in alternative embodiments, the reference generator of the power management unit 120 can comprise the digital to analog converter which allows the generated reference voltages to be programmable.

Various implementations of the reference generator of FIG. 5 or FIG. 6 may include a diode to provide a reference current. At different temperatures the performance of the diode may vary. Thus, a temperature sensor provides a means to measure the temperature and correct for the performance of the diode within the power management unit. These temperature measurements can be used outside of the power management unit. For instance, the temperature measurements can be provided to the user via the transmitter to calibrate the signal obtained from the sensing element 160. The temperature measurements themselves are also useful indications (e.g. of the glucose concentration within a patient's body) because in some cases, a decrease in glucose level causes cooling of the peripheral skin and tissue.

The power detector of FIG. 5 or FIG. 6 can be used to measure the incoming power signal level and determine whether the implant is underpowered, properly powered, or overpowered, and report the power data to the external transmitter as part of packet data.

A control logic can be implemented within the power management to execute the tasks of the regulator, reference generator, power detector, temperature sensor, and voltage limiter, for instance a processor. The control logic can, in various embodiments, execute tasks for the sensor signal acquisition unit or communication unit.

In embodiments, the power management unit may include a voltage limiter. A voltage limiter can massage power to be more usable by the multianalyte monolithic integrated circuit. For instance, the voltage limiter can protect the system from over-voltage by using different methods including by sinking more current and hence reducing the supply voltage from the transmitter. In accordance with some embodiments of the invention, an implantable multianalyte monolithic integrated sensor circuit 1 can be powered through a power management unit using the two-wire flexible connector and the data transmission can use a low-power wireless communication scheme (e.g., Bluetooth Low Energy, ANT, Zigbee) as implemented through the optional interface unit.

As shown in FIG. 5 or FIG. 6, the sensor signal acquisition unit 130 can include an oscillator, potentiostat(s), an analog to digital converter (ADC), and a multiplexer (MUX) (as well as an optional digital to analog converter (DAC) to externally control electrode redox potential). The oscillator can be used to provide an accurate and clean reference clock for the implant that is used by the communication unit, power management unit, and the sensor signal acquisition unit.

In one embodiment a potentiostat is connected to on-chip electrodes as shown in FIG. 5. The potentiostat maintains a fixed defined voltage between working electrode(s) and a reference electrode while providing current through a counter electrode. Each working electrode can have a dedicated potentiostat to maintain the appropriate voltage between the working and reference electrode and minimize crosstalk. However, a shared potentiostat is also workable.

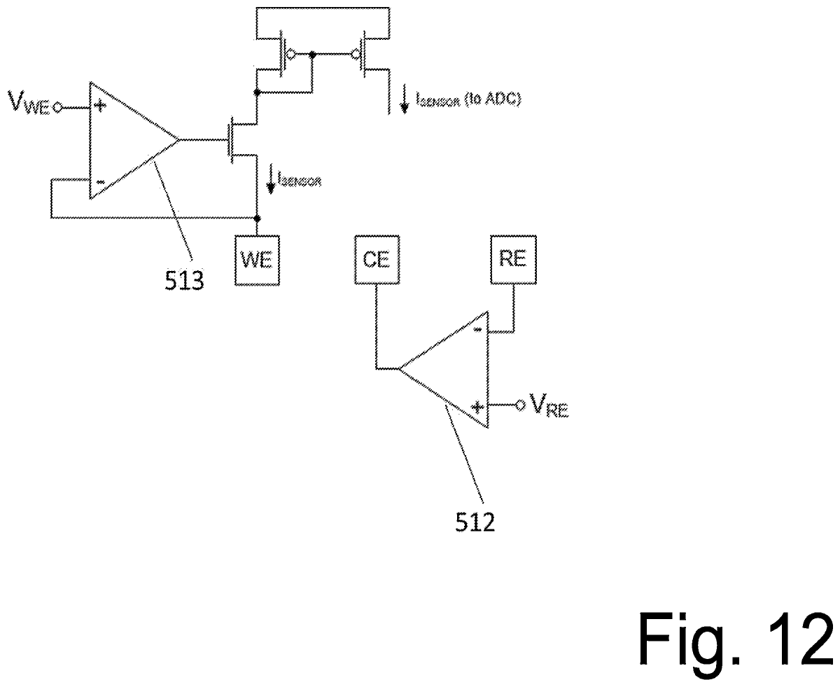

An embodiment of wiring schemes of the sensor signal acquisition unit (in particular the potentiostat) connected to working, counter, and reference electrodes can be seen in FIG. 12. At the top, an analog front end (AFE) of a model working electrode includes an amplifier 513 which can be used in negative feedback to maintain the potential at the working electrode, V W E, while buffering the sensor current I_{SENSOR} to the ADC. The sensor current I_{SENSOR} is inversely proportional to the resistance of the working electrode which varies with the glucose concentration in the blood. The bottom shows an amplifier 512 which can be utilized to form negative feedback between reference and counter electrodes. The reference electrode's potential is maintained at VRE through the negative feedback, while the amplifier provides counter electrode current. The AFE and the amplifier 512 may be collectively referred to as the potentiostat. Therefore, the potentiostat senses the changes in the resistance of the working electrode, by outputting a current which is inversely proportional to the varying resistance of the electrode. This method allows for independently controlling the potential difference between working and reference electrodes in a multi-analyte sensor where there are several working electrodes for sensing different analyte.

The potentiostat is continuously powered by the battery 212 of the transmitter 2 (shown in FIG. 5) via the connector 3. By continuously powering the potentiostat, the potentiostat is able to maintain the potential of the working electrode continuously. After installation of the battery 212 into the transmitter 2, it may be required to calibrate the system of FIG. 2. However, after the initial calibration, there is no need to further calibrate the system before the battery 212 is drained up. Therefore, the system of FIG. 2 is able to continuously measure the glucose level of a patient during the lifetime of the battery 212. The measurement frequency may be from once every tens of seconds to once every few minutes, and may be adjusted at the transmitter side (e.g., by a user using the smartphone 4 wirelessly connected to the transmitter 2). Note that it is still possible to supplement the initial calibration with second, third, or further calibration points to enhance accuracy or in the event the user believes deviation has occurred.

The CMOS sensing circuit with the potentiostat is placed at a depth of 0.3 to 10 mm and ideally at a depth of 2-3 mm beneath the patient skin. The 2-3 mm depth has been found to allow the potentiostat to generate a superior signal indicative of glucose concentration. The 2-3 mm depth also significantly shortens the communication distance between the potentiostat and the transmitter 2. It would be understood that the potentiostat may be placed at a depth of 1 to 5 mm beneath the patient skin or a depth of 0.3 to 10 mm beneath the patient skin.

With further reference to FIGS. 5 and 6, the potentiostat can be connected within 9, 5, or 2 millimeters of the entirety of at least three working electrodes WE1, WE2, and WE3. This means that, for example, when WE1 is arranged closest to the potentiostat within the working electrodes WE1 to WE3 and WE3 is arranged furthest from the potentiostat, the distance between the potentiostat and all the area of the furthest electrode WE3 is within 9, 5, or 2 millimeters. Alternatively, the potentiostat is connected within 0.5 millimeter or 400 microns to the entirety of working electrodes WE1, WE2, and WE3. The short connection distance between the working electrodes and the potentiostat allows for minimal electronic signal travel, which in turn improves the signal quality and reduces the power consumption of the system of FIG. 2. It should be pointed out that other continuous glucose monitoring systems have substantially larger distances between the potentiostat and the working electrodes. For example, the Dexcom G6 contains a wire that is at least 15 mm long, and thus, the distance between the entirety of the working electrode and the potentiostat of this device is far greater than those proposed in the instant disclosure.

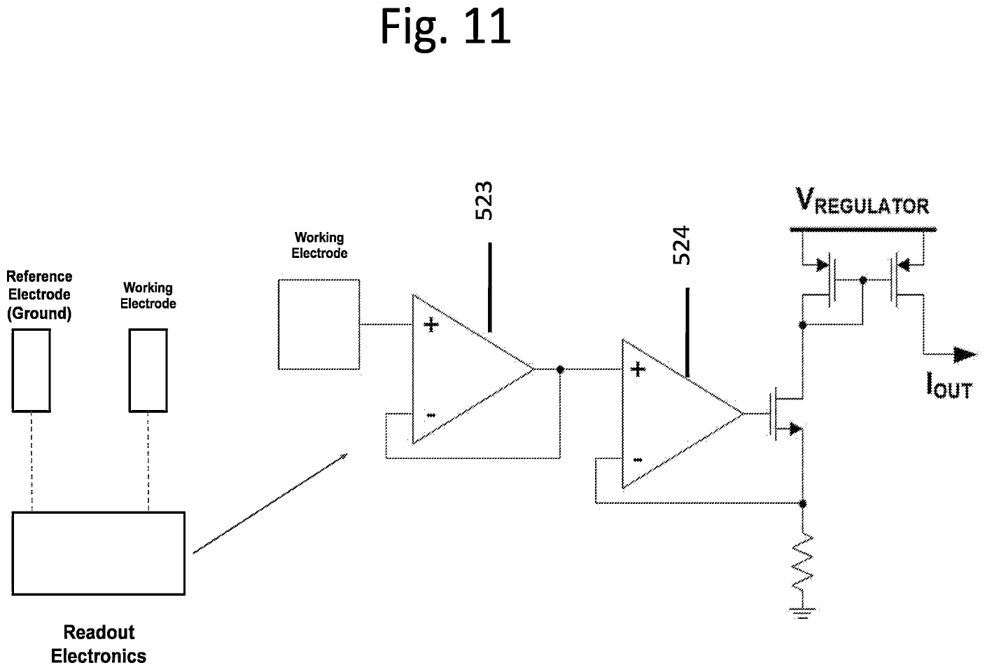

In one embodiment a potentiometric sensor is used alternatively/additionally to a potentiostat. An exemplary potentiometric sensor can be seen in FIG. 11. The WE is connected to a buffer amplifier 523 which in turn is connected to a voltage-to-current converter 524. Hence, the WE potential is translated into sensor current which is then fed to the same ADC that converts the amperometric sensor signal into a digital signal.

C) Multiplexing

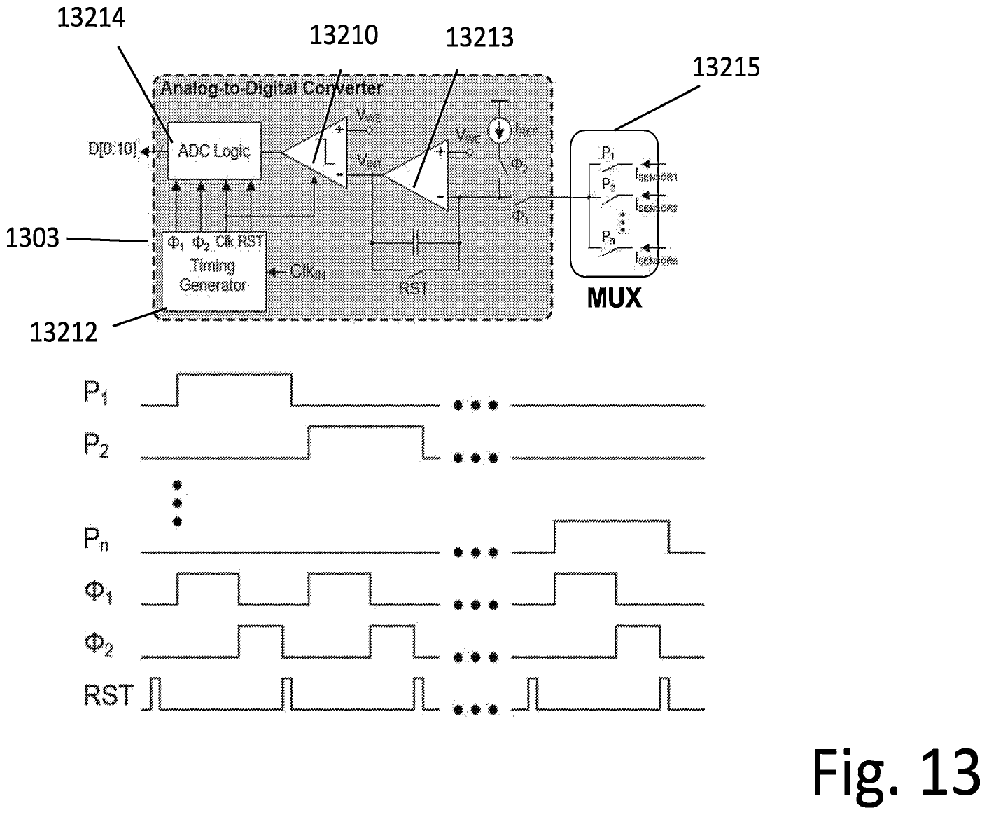

A dual slope ADC can be used to directly convert the sensing element current coming from the potentiostat into the digital domain as shown in FIG. 13. The ADC can, for example, have an 8-12 bit resolution. The ADC converts the potentiostat current into digital data values. Since in-vivo glucose readings don't change rapidly, each electrode pairing can be sampled every millisecond to once every 100 milliseconds, but preferably once every 10 milliseconds (100 samples/second). An ADC with high sampling rate (1K samples/s) can be utilized to sequentially digitize the signal from multiple electrodes as well as the temperature sensor.

An embodiment of an ADC of a sensor signal acquisition unit can be seen in FIG. 13. An ADC 1303 is depicted which can convert a sensor signal (e.g., sensor current) into a digital signal (e.g., bit stream) using a mixed-mode circuit consisting of an amplifier 13213, a comparator 13210, a timing circuit 13212, and a control and logic unit 13214. A reset switch RST is used to reset the system before conversion of a new signal from the sensor. A capacitor is used to accumulate the sensor current over a given time, after which it is converted to a digital signal using the rest of the circuit. The reset switch discharges this capacitor to restart the process. ADC 1303 is fed by a signal from a multiplexer 13215 which in turn is connected to the signals from all the on-chip sensors. The multiplexer is fed by control signals P₁, P₂, . . . , P_{N} which are used to control which sensors' signal goes to the ADC. The control signals are generated by the on-chip control logic.

A multiplexer (MUX) 13215 in the sensor signal acquisition unit can connect signals from multiple working electrodes on the monolithic integrated circuit to the ADC to be digitized in a time-multiplexed fashion. Each working electrode (sensor) current (I_{SENSORn}) is connected to the ADC at the time period Φₙ. In accordance with some embodiments, to support multi-analyte sensing without an excessive increase in power consumption, resource sharing can be enabled across the sensor signal acquisition unit by the multiplexer. In some embodiments of the disclosure, each individual working electrode can be controlled by a dedicated potentiostat while an analog-to-digital converter can be shared among all potentiostats through time division multiplexing in which the digitization period is divided among some or all the working electrode-potentiostat pairs. During each time slot, the output of one working electrode-potentiostat pair is digitized. In accordance with some embodiments of the disclosure, the sampling rate can be set to a rate that is well above the rate at which relevant physiological body changes occur to avoid sensed signal loss. Normally, the ADC can operate at a much faster rate than that of the physiological signals, hence such multiplexing doesn't create any loss of needed data.

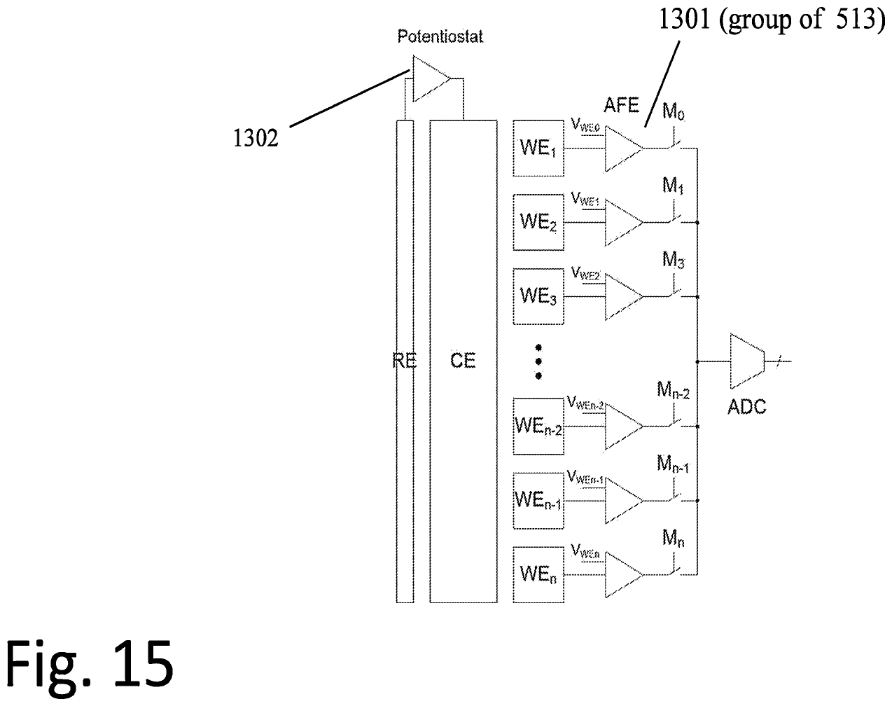

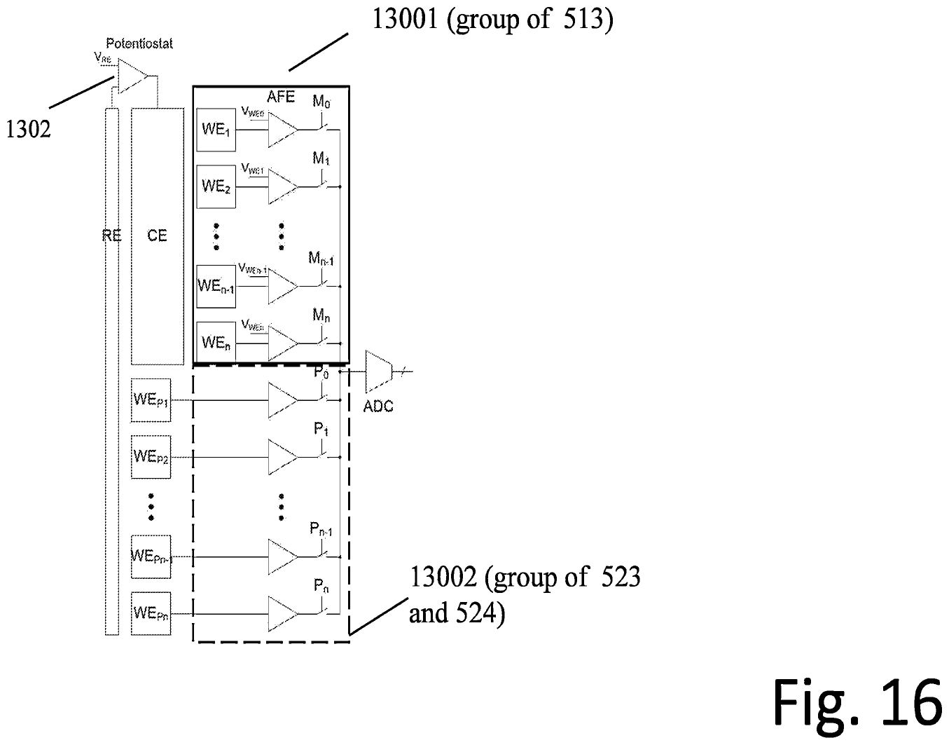

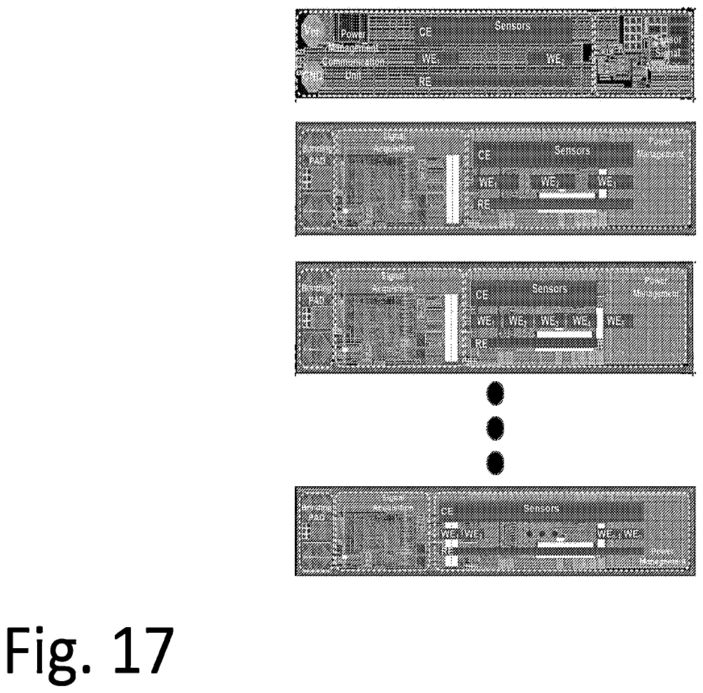

An embodiment of multiplexing sensing circuitry can be seen in FIG. 15. FIG. 15 describes an embodiment of sensing circuitry (e.g., the integrated sensing element 160 and the sensor signal acquisition unit 130) using minimal chip area through time multiplexing and sharing of counter (CE) and reference electrodes (RE) for the case of multi-analyte sensing using amperometry only. Multiplexing and sharing of counter (CE) and reference electrodes (RE) is shown. The time-multiplexing is achieved by using the multiplexer 13215 and the ADC 1303 and multiple separate working electrodes (WE₁, . . . , WEₙ) with respective analog front-ends (AFE). Multiple AFEs 513 (e.g., based upon op-amps 513) of FIG. 12 can be used as each AFE as each AFE with the grouping denoted as 1301. The multiple working electrodes can be managed each by multiplexer connections M₁-Mₙ to feed their corresponding signals (data) to the ADC in a time-multiplexed manner. The redox potential at each WE is controlled by the corresponding V_{WE} voltage. This enables the design to have different working potentials for different working electrodes, thus enabling a wide variety of analytes to be detected. FIG. 16 shows a scheme in which multiple types of detection methods can be combined on the same monolithic integrated sensor circuit 1. In an exemplary embodiment, multi-analyte amperometric and the voltametric (potentiometric) schemes are combined. Multiple separate working electrodes (WE₁, WE₂, . . . , WEₙ) with respective analog frontends (AFE) can be used for amperometric sensing whereas the multiple separate working electrodes (WEp₁, WEp₂, . . . , WEpₙ) are used for potentiometric (open-circuit voltage) sensing. The multiple working electrodes can be managed each by multiplexer connections M₁-Mₙ for the amperometric sensors and by P₁-Pₙ by the potentiometric sensors. The circuit element labeled as 13001 represents a set of analog frontends (AFE) for the amperometric sensors which comprise of a part of the Potentiostat circuit (amplifier 513) whereas the circuit element labeled as 13002 represents a set of analog frontends (AFE) for the voltametric sensors which comprise the potentiometric circuit elements (amplifier 523 and 524).

D) Temperature Sensor

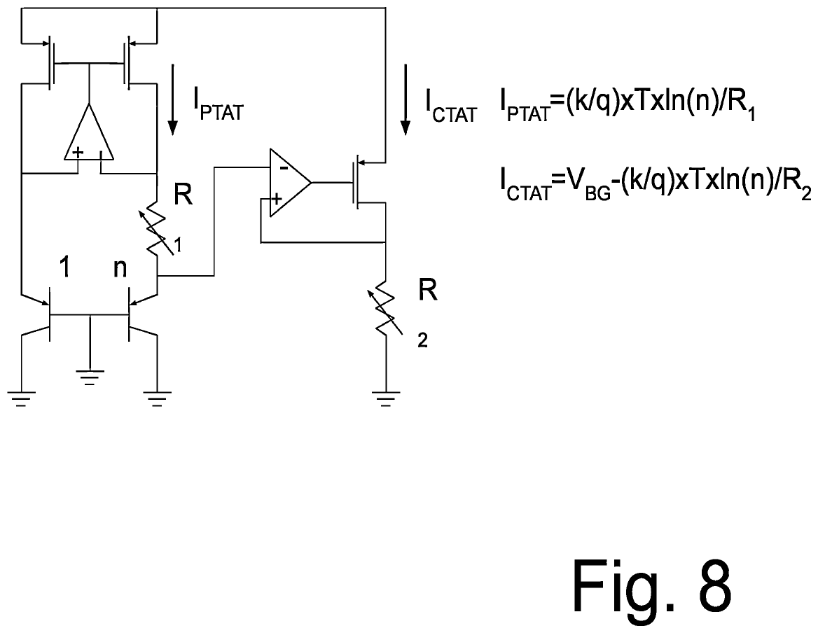

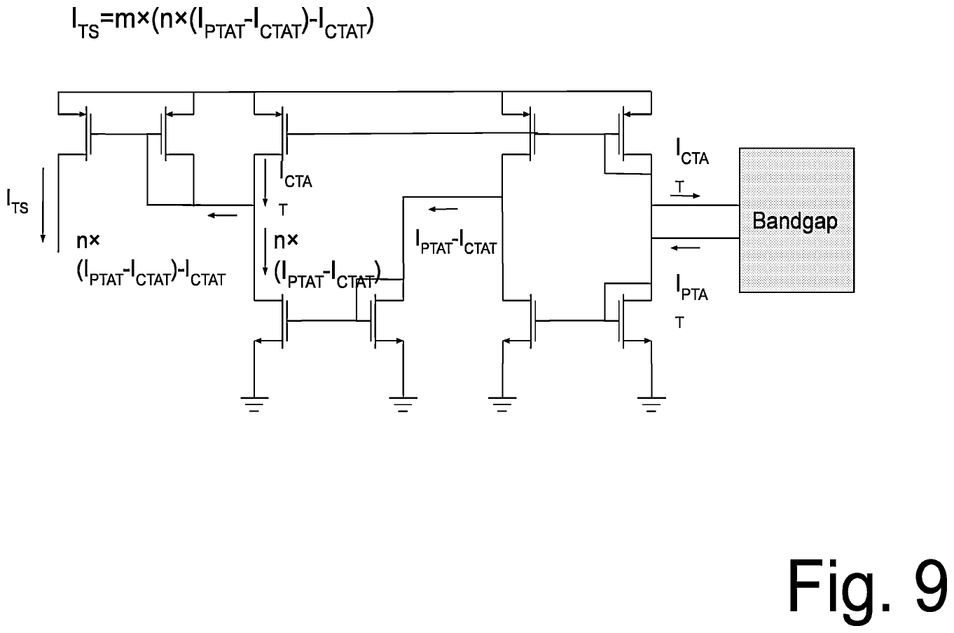

A temperature sensor may be included in embodiments of the power management unit of FIG. 5 or FIG. 6. An implementation of a temperature sensor can be seen in FIG. 8 and FIG. 9. FIG. 8 shows a temperature sensor circuit. A bandgap circuitry generates currents proportional to absolute temperature (PTAT) and current complementary to absolute temperature (CTAT). As shown in FIG. 8: k—Boltzmann, q—charge e, T—temperature, ln—natural log, n—ratio of the 2d area between the two bipolar transistors.

FIG. 9 shows an amplification scheme for the temperature sensor circuit current to increase sensitivity. To increase the dynamic range of the temperature sensor, the offset of the temperature dependent current is removed so that at the temperature range of interest the current varies between a value close to zero and the full range of the ADC. Multiple current amplification stages bring the range of the current variation close to the ADC dynamic range.

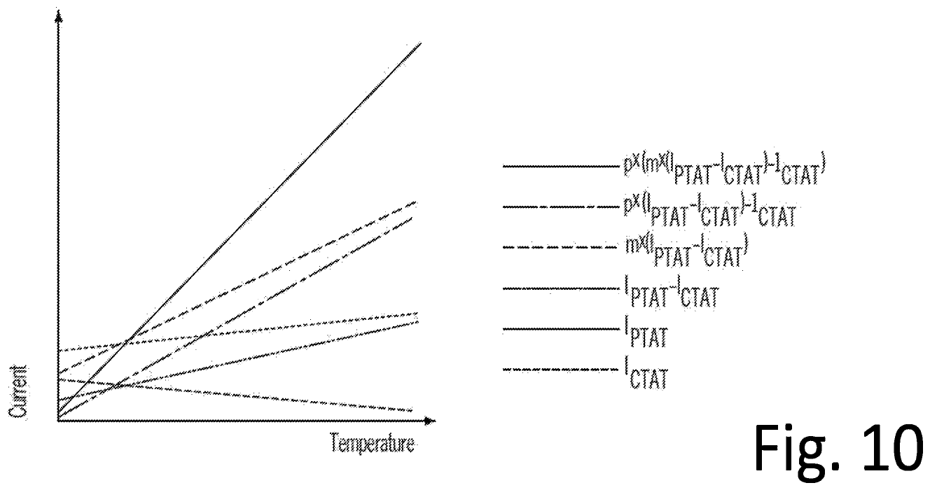

FIG. 10 shows a mathematical representation of the amplification method implemented.

Another method to implement the temperature sensor is via a resistance temperature detector (RTD). In this case, a thin filament like electrode can be made on top of the CMOS device (e.g., on the top layer) using a material with good temperature sensitive resistance. The circuit underneath can read the resistance and hence any change in temperature. An example of the RTD is a Platinum based RTD. Since the system enables fabrication of multiple electrodes on the CMOS substrate, fabrication of such a temperature probe is simple and can be done together with the fabrication of the electrochemical sensors. The RTD probe can be coated with a thin insulator layer afterwards (e.g., thin Silicon Nitride layer).

E) Programmable Potential

The redox potential at each WE can be controlled by the corresponding V_{WE} voltage (which may also be referred to as a reference voltage). This enables the design to have different working potentials for different working electrodes, thus enabling a wide variety of analytes to be detected. In particular, each of these op-amps 513 is connected to the desired redox potential (e.g., V_{WE1}, V_{WE2}, V_{WE3}, and so on) for that WE. These voltages can be either internally generated as a fixed value for each application (e.g., using on-chip reference voltage generators), or can be generated using a Digital to Analog Converter (DAC) so that they can be programmed (FIG. 14) by the user from a given set of values to allow for programmability in choosing what analyte to measure. The user input of given set of values (also referred to as “command”) may be transmitted from the transmitter 2 to the implantable multianalyte monolithic integrated sensor circuit 1 via the connector 3.

An example of the DAC circuitry can be seen in FIG. 14 including a resistor ladder comprising 2^{m} resistors connected between the voltage regulator output and the ground to form an m-bit DAC capable of generating 2^{m} different voltages. These voltages are connected to each working electrode through a set of switches controlled by Dᵢⱼ, where i represents WEᵢ and j is between 1 and 2^{m} for selecting the desired voltage level. For each working electrode there are 2^{m} dedicated switches.

As shown in FIG. 5 and FIG. 6, in some embodiments of the disclosure, the implantable monolithic circuit can include an electrochemical integrated sensing element that comprises multiple working electrodes (e.g., a detection reaction can occur at this electrode), a single counter electrode (e.g., can be used to balance the current generated by working electrode) and a single reference electrode (e.g., to provide a stable voltage reference signal inside the body). The electrochemical integrated sensing element transduces changes in analyte concentration into changes of electrical current. In an embodiment the integrated sensing element includes three working electrodes, one counter electrode, and one reference electrode. The sharing of a single counter and a single reference electrode does not create crosstalk as the current in each potentiostat can be determined by the corresponding working electrode. However, in a separate optional embodiment one counter and one reference electrode can be used corresponding to each single working electrode. In an example, the desired redox potential may be between −1.5V to 1.5V. In the event that the resistor ladder of FIG. 14 is connected between a positive voltage and a negative voltage, the desired redox potential may be between −1.5V to 1.5V.

To directly detect glucose, working electrode(s) can be functionalized with GOx hydrogel (the chemistry and deposition of GOx hydrogel is discussed more below). In an embodiment, indirect glucose sensing through differential Oxygen sensing can be implemented by using one working electrode to measure background O₂ using a non-enzyme loaded hydrogel, while another working electrode is functionalized by GOx hydrogel to measure left-over Oxygen from the Glucose-Oxygen reaction (Oxygen consumed by the enzyme). The difference between these two Oxygen concentrations can indicate the glucose concentration. The GOx functionalized electrodes can be intentionally placed apart (by having WE, in between) to minimize crosstalk. On the electronics side, multiple potentiostats (n) can be included to control the sensors. For example, GOx sensor working electrode can be held at +0.3V-0.6V with respect to the reference electrode while an O₂ sensing working electrode can be held at −0.3V-0.5V with respect to the reference electrode (oxygen detection potential). The current from the potentiostats and the temperature sensor can be digitized by the shared on-chip ADC in a time multiplexed manner.

### Multianalyte Monolithic Integrated Sensor Manufacturing

There are different ways the presented system can be manufactured. FIG. 4 shows one manufacturing scheme in which the sensors design is sent to a CMOS foundry which fabricates CMOS wafers (e.g., 8-inch wafers in TSMC 180 nm process). The wafers are then post-processed in a cleanroom to replace the top metal in 180 nm CMOS from Aluminum to Platinum (more suitable for sensing). Next, the wafers are thinned and cut to create individual sensor chips. The sensor chips are bonded with a flexible electronics substrate in the form of flexible PCB panels. One method to achieve this bonding is flip-chip bonding. Another method is wirebonding. After this, the sensors are coated with multiple chemistry layers (e.g., two different enzyme layers followed by polymer membranes) at the panel scale using a combination of droplet deposition and spin coating. After testing the sensors at the panel, the panels are cut (e.g., using lasers) to enable the separation of each sensor from the panel. The sensors are then attached to a transmitter that is independently assembled and validated with test sensors. After that, the assembled sensor-transmitter pair is loaded in an applicator and the entire device is sterilized using a suitable sterilization method like electron beam sterilization, Ethylene Oxide sterilization etc.

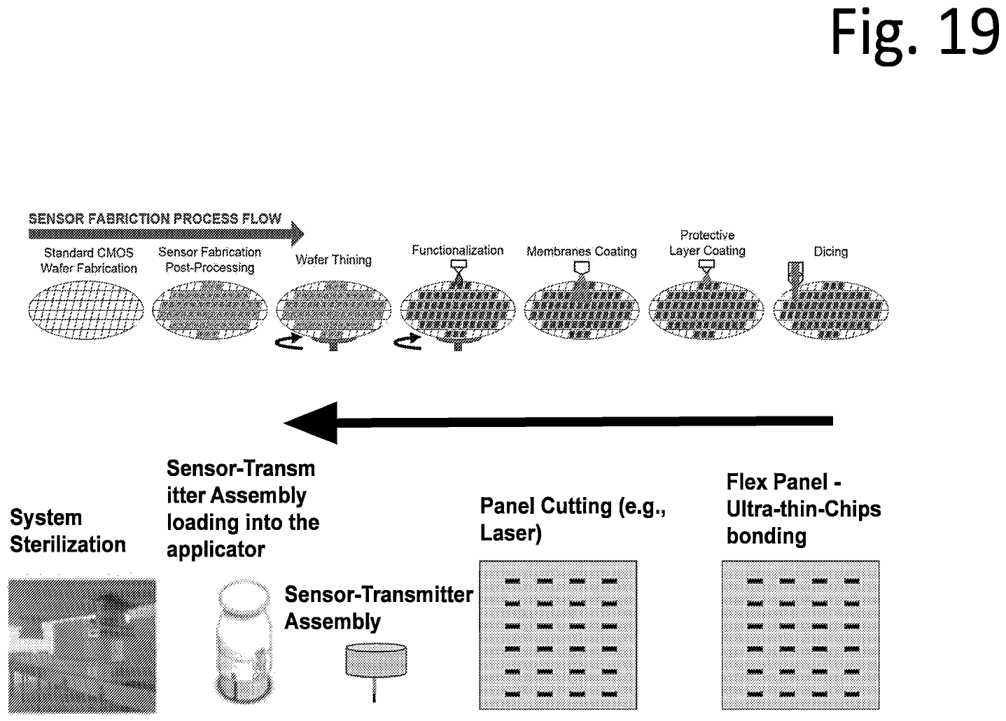

In a different embodiment as shown in FIG. 19, the CMOS wafers are post-processed followed by thinning and chemistry coatings and protective layer coating(s) followed by dicing. These functionalized sensors are then bonded with a flexible PCB panel while avoiding damage to the chemistry by avoiding high temperature or pressure in the sensing region. The flexible PCB panels are cut to separate the individual sensors which are then connected to a separately assembled transmitter. The sensor-transmitter assembly is then loaded into an applicator and the complete assembly is then sterilized using a suitable scheme (e.g., electron beam sterilization, ethylene oxide sterilization).

A) Metal Deposition

The process for forming the functionalized electrodes of the multianalyte monolithic integrated sensor circuit after receipt of a wafer from a commercial foundry (e.g., TSMC) is now described in greater detail.

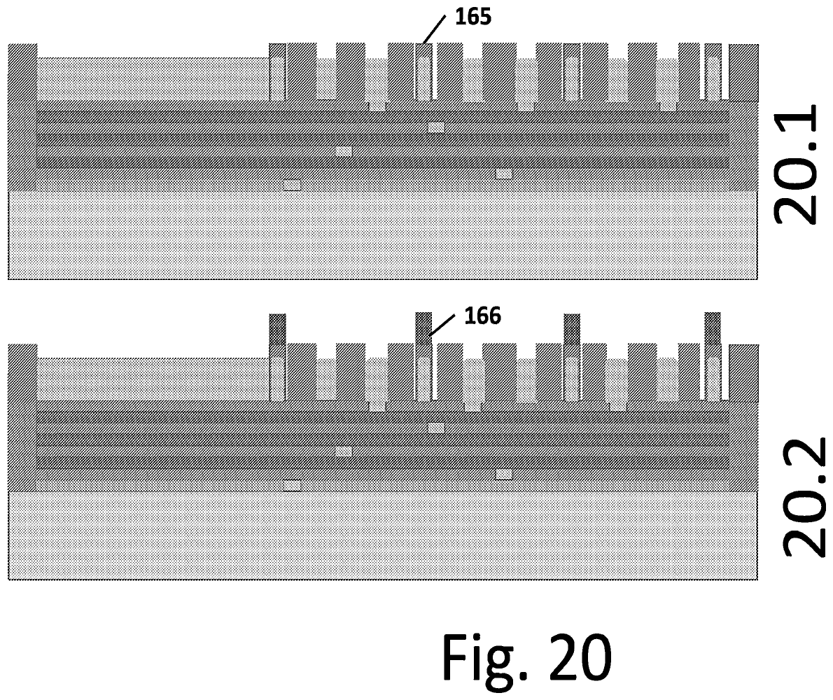

FIG. 20 shows two different embodiments of how the CMOS integrated sensing circuit may appear after CMOS fabrication. The view in FIG. 20.1 Shows a scheme in which the top insulation only uses the standard solid-state insulating layers like Silicon Oxide and Silicon Nitride (layer 165 in FIG. 20), while the view in FIG. 20.2 shows an embodiment in which a polymer layer is added in addition to the solid-state insulating layers (the combination is labeled 166 in FIG. 20) to make the well walls higher to increase the separation between the adjacent electrodes to minimize interference. The top insulator 118 can be either 165 or 166 as shown in FIG. 20.

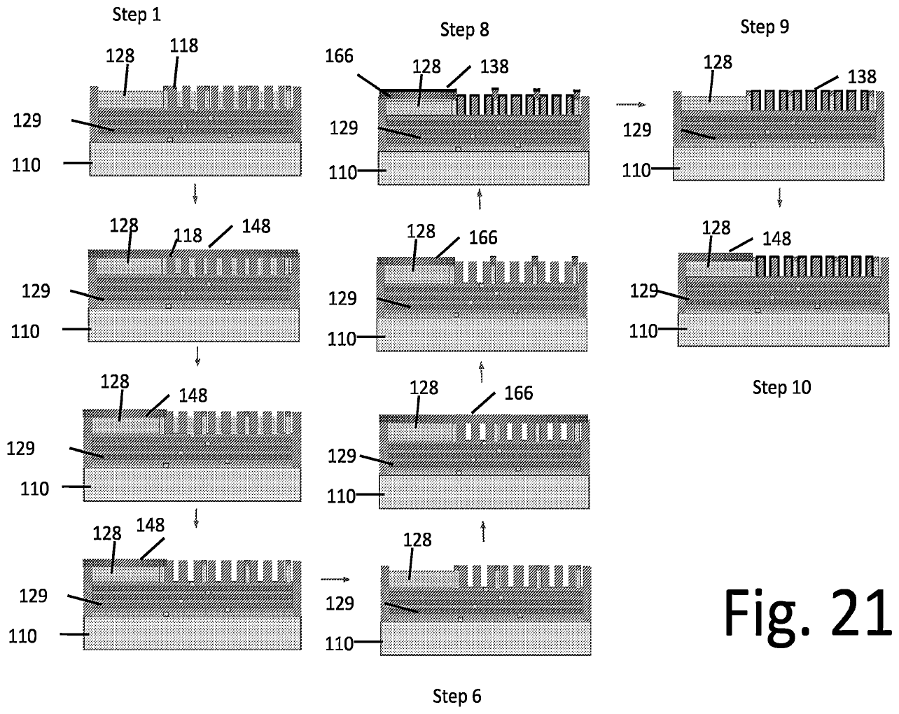

The wafer (diced or un-diced) as being processed is hereafter referred to as a monolithic circuit precursor. An example of a wafer or circuit precursor can be seen at FIG. 21 step 1. FIG. 21 at step 1 is a side cross-section schematic view of an integrated sensor circuit precursor, showing a substrate 110, a metal-insulator-metal stack 129, a top metal 128, and a top insulator 118.

Note that the top metal can be thick in high frequency CMOS processes. In some cases, a more suitable material can be coated on the top metal without etching it. For some other cases, a first step of post-processing involves removal of this top metal layer for better control of the morphology of the more suitable material deposited afterwards. This etching can be achieved by using wet etching (e.g., using a mixture of Nitric acid and Phosphoric acid) or dry etching (e.g., Chlorine based RIE Plasma).

After receipt of a precursor of an implantable monolithic sensor circuit from a commercial foundry or after post-processing for removing, for example, a thick top layer of top metal from selective areas (using lithography and etching), lithographic (e.g., photolithography) patterning can be done to expose the sensing element electrodes while covering the rest of the wafer with a suitable material (e.g., photoresist). Note that, for some applications, this patterning can be achieved using custom stencils, i.e., without lithography. Ins some other applications, this patterning can be achieved using electron beam lithography without the use of a photomask. The choice of the patterning method depends on parameters like required resolution, cost, and scalability.

In FIG. 21 step 2 a precursor is coated using lithography with a resist (e.g., photoresist) 148. In one embodiment, the top first layer metal 128 of a Metal-Insulator-Metal (MIM) structure is Aluminum. Underneath the top metal is the further Metal-Insulator-Metal (MIM) structure 129 found in CMOS devices. A silicon substrate is at 110. Note that several electrodes can be isolated from each other by top insulation 118 (Top insulation in CMOS process is often a stack of Silicon Nitride layer on top of Silicon oxide layer). Top insulation can be further augmented by an additional layer (e.g., Polyimide layer). This insulation also separates the contact pads 170 from the sensing element electrodes 160.

In photolithography, after photoresist deposition, it is patterned to provide a desired pattern (e.g., opening) in select areas for further processing. In FIG. 21 step 3, the photoresist 148 is patterned by exposing to UV and developing in a suitable developer (e.g., AZ300MIF).

In FIG. 21 step 4, a precursor is etched to remove the top metal 128 from sensor electrodes in areas where the photoresist is removed by patterning in the previous step, leaving the area which was protected by photoresist with the top metal and further exposing a second further layer of the Metal-Insulator-Metal (MIM) structure 129 having vi as the connect the top metal with the lower metal in other areas where the top metal is etched. The silicon substrate remains at 110. Electrodes remain isolated from each other by top insulation 118.

This patterning can be followed by another patterning to control the areas for the deposition of a desired material (e.g., suitable metal stack); for example, a Ti (Titanium) or TiW (Titanium-Tungsten) intermediate layer of small (e.g., 20 nm) thickness as the adhesion layer (and to avoid corrosion of an underlying Aluminum layer if the Aluminum layer is not removed) can be deposited followed by deposition of relatively thicker (e.g., 100 nm) of Platinum (or any other noble metal or corrosion resistant conductive alloy or other material that can be used in sensing). Physical vapor deposition (PVD) e.g., sputtering, e-beam deposition, and thermal evaporation, chemical vapor deposition, electroplating, and electroless plating are different methods that can be used for thin film deposition. Sputtering can form a relatively rough surface compared to e-beam or thermal deposition, both of which result in smoother electrodes. Moreover, after selectively removing the top metal from certain regions, that photoresist can be removed and a second photolithography step can be done to provide a cleaner pattern for patterning the subsequent metal deposited using PVD. This second photolithography and patterning is shown in steps 5-8 in FIG. 21.

In the most preferred embodiment, the top metal is protected in the contact pads area during postprocessing using a protective layer like photoresist. This keeps it clean and thick to enable easier connections with the flexible connector 3. In some cases, the top metal can be etched from the contact pads area as well and replaced with a suitable metal stack (e.g., Ti, Palladium, Gold), if it suits the bonding process with the flexible connector 3. In some other cases, a more suitable metal (e.g., via ENEPIG or ENIG) structure can be formed on top of the contacts pads to make it easier to bond it with the flexible connector 3. This process can be done before or after other postprocessing steps.

To achieve higher surface area and to enhance bonding between the sensing element and the subsequent chemistry layers, the metal surface can be designed to have rougher finish (as compared to smooth or mirror finish). This is achieved by controlling deposition method (e.g., electron beam deposition, thermal evaporation, chemical vapor deposition, sputtering), depositional environment (e.g., pressure), and deposition energy. In an embodiment, sputtering at 30 mTorr pressure and 100 W DC power generates a metal coating with a high surface area for a planar geometry. Moreover, such metal layers also improve the adhesion between the sensing electrodes and the subsequent chemistry layers.

After selective patterning using lithography, a precursor is coated with a desired material (e.g., suitable metal) in FIG. 21 step 8. A thin layer of desired material (e.g., Platinum) 138 is coated using a thin-film coating method. A substrate 110, a metal-insulator-metal stack 129 and a top insulator with an optional additional insulator/protection polymer layer 118 remains. The top insulation electrode isolation region remains protected by photoresist 148.

The use of patterned electrodes with one or more pillars can be helpful as noted above because strength of the sensing element signal can be proportional to surface area of the electrode. As noted above patterned electrodes can be formed by post-processing in an embodiment. In post-processing, it is possible to lithographically form the pattern for electrodes in a lithographic material (e.g., photoresist) while covering the rest of the chip with the lithographic material. This is to be done before the deposition of a suitable metal stack. As an example, AZ 5214E resist can be spun at 3000 rpm, baked at 95 degrees Celsius for 5 minutes, and exposed using i-Line (e.g., 365 nm UV radiation) exposure in an MA6 mask aligner for 2 seconds. LOR resist can be used to help with liftoff. Image reversal can also be used for this purpose. In this case, a post-exposure bake at 110 degrees Celsius for 2 minutes is performed followed by a flood exposure in MA6 for 3 seconds. For both positive and negative patterns, the resist can be developed in a developer (e.g., AZ300). This can be followed by sputtering of Ti (e.g., 20 nm) and/or TiW (20 nm) followed by Pt (100 nm). Sputtering parameters are optimized to achieve the desired morphology of the coated material (e.g., Pt). After sputtering, a conformal coating is achieved. Solvent Lift-off can then be performed (e.g., dipping sensors in acetone for 30 minutes) to remove metal from unwanted areas and only keep those on sensing element electrodes. Alternatively, materials can first be deposited everywhere and then etched with appropriate wet and/or dry etching methods.

A next step of the post-processing can be lift-off to remove metal layers from the unwanted regions where there is a photoresist layer coated above the CMOS chip. This is achieved by soaking the coated devices in solvents. Alternatively, unwanted metals from coated devices can be etched in appropriate etching solutions (e.g., in aqua regia) or using dry etching (e.g., Gas Plasma Etching). In this case, the metal is first deposited and then coated with a photoresist or a protective layer in certain areas while opening up the remaining areas from where the metal is then removed (e.g., using wet or dry etching).

In FIG. 21 step 9 a precursor is cleaned to remove the photoresist 148 and excess deposited material and leave deposited material only in desired places (e.g., on integrated sensing element electrodes). A substrate 110, a metal-insulator-metal stack 129, a thin layer of desired material 138 and a top insulator with an optional additional insulator/protection polymer layer 118 remains.

It is possible to perform another lithography followed by silver deposition, liftoff and Chlorine exposure through wet solution (e.g., Ferric Chloride) or dry plasma (e.g., Chlorine Plasma) to create silver-based reference electrodes (e.g., Ag/AgCl). Ag/AgCl reference electrodes are more suitable for some applications (e.g., open circuit potential measurements).

It is also possible to create polymer structures around the sensing element electrode area to create further isolation or to improve chemical functionalization. An example is the use of insulating material to cover the unexposed parts of the top metal. This layer may consist of Silicon Oxide and Silicon Nitride insulating layers found in standard CMOS process as well as additional insulating/polymer layers (e.g., polyimide) to protect the underlying circuitry. Polymer walls around the sensor can be used to act as ‘well structures’ as well as ‘adhesion promoting structure’ as some functionalization materials (e.g., Serum Albumin based Hydrogel) adhere better to an activated polymer surface than to a Silicon Nitride insulation structure. In some cases, such structures can be provided by the CMOS foundry or a similar foundry as part of the fabrication process. For example, polyimide structures can be provided to the end-user by the CMOS foundry and can work as adhesion promoters for some applications.

In accordance with some embodiments of the disclosure, the sensor electrode surfaces can be activated (e.g., with glutaraldehyde or air plasma, oxygen plasma, or argon plasma) prior to functionalization layer (e.g., hydrogel) deposition. This activation can help with adhesion of the sensor chemistry with the sensor or the previously deposited chemistry layers. Surface structures and/or modifications can also act as grafts for a functionalization layer (e.g., hydrogel) and result in a stronger adhesion and/or chemical interaction between the gel and the sensor electrodes.

Additionally, in optional embodiments of the disclosure, a layer that can limit sensor response to substances that interfere with sensor operation can be applied to the surface of one or more of the electrodes before coating a functionalization layer (e.g., a hydrogel). For example, a layer of thin polymers (e.g., polyaniline) can be formed on the sensor by spinning and UV/electron beam crosslinking. For example, a layer of poly-phenylenediamine polymer can be coated on electrodes surface using electrochemical deposition or UV crosslinking, before or after the enzyme coating. This allows the sensor to not react to ascorbic acid or acetaminophen which otherwise can create a false signal on platinum electrodes.

In another embodiment, parts of the CMOS device can be coated with a protective material while keeping the rest of the areas open for further processing (e.g., chemical coatings). Such a layer can be patterned using photolithography or other patterning methods. FIG. 21 step 10 shows such a coating 148 on the CMOS precursor covering the contact pads. It can be used to cover the sensing element electrodes as well as any other part as required by the next processing.

### A.1) Metal Deposition Structures

In one embodiment, the individual electrodes can be defined by openings in a top passivation layer (e.g., 118 in FIG. 21) of the CMOS fabrication process. An integrated design wherein electrodes are defined by the topmost Aluminum metal layer in the CMOS process can be used for electrochemical sensing. However, since Aluminum may not be suitable for stable electrochemical sensing due to corrosion, lithographic post-processing can be performed to replace or coat it with a noble metal (e.g., platinum, gold, silver) for working electrodes and counter electrodes and Pt/PtOx (more stable reference than Pt, easier to fabricate than Ag/AgCl) for reference electrodes. These post-processing steps are more specifically discussed below. Note that the reference electrode can also be coated with a noble metal instead of Pt/PtOx. The strength of the sensing element signal can be proportional to surface area of the electrode and the effective signal strength can be increased by utilizing patterned or non-planar electrodes instead of conventional planar electrodes. The working electrodes (and optionally counter and reference electrodes) can be designed to have pillar structures by patterning the top metal and passivation layers to enhance sensitivity as well as improving adhesion between the solid-state sensor and the hydrogel. Such structures can be formed using a semiconductor fabrication process, by post-processing or by a combination of both. Pillars created during a CMOS manufacturing process can have a size and spacing (determined by the CMOS process) of about 0.25 μm-25 μm and preferably 2 μm-5 μm and height of about 0.1 μm-10 μm and preferably 24 μm-5 μm (theoretically resulting in about 2-3× increase in sensor signal). Post-processed pillar structures are discussed below. Both foundry and post-processed pillars share similar dimensions and can be rounded or square. In accordance with some embodiments of the disclosure, the pillars can be partially or completely etched to form a more planar structure. When completely etched, the pillars are totally removed. However, the resulting insulation structure on the surface results in surface texturing after deposition of metal. When partially etched, pillars are thinned down and are shorter in height and possibly width.

An example of surface patterning which can be accomplished via foundry semiconductor processes and a degree of post processing is a hole-based reverse pillar design or a pillars-based design.

In a hole-based reverse pillar design, the electrodes are designed as metal array and the gap is filled by the top insulator. The metal is etched in post-processing to create holes in the insulator surface. This method uses the small features size available in the CMOS process to form structures that enable the formation of such high surface area structures with simple postprocessing steps like wet etching. The reverse design can be done to achieve pillars of insulating materials fill the gap between metal mesh. When the metal is etched in post-processing, the insulator pillars remain to form a pillar structure.

These patterned electrodes are different than those reported elsewhere. These electrodes are formed by using the design rules and materials available in the CMOS process itself (e.g., insulator), instead of having to form all the patterning on a silicon substrate afterwards. This simplifies the design and enables scalable and more controlled structures. This also enables the use of smaller features in advanced CMOS processes (e.g., 3 nm process) as those are achieved by advanced photolithography methods not available in cleanrooms outside of the advanced CMOS foundries. Moreover, this simplifies the post processing steps which are otherwise difficult to match with the features and control available in the CMOS fabrication process.

The size and shape of the electrode structure can be selected based upon the sensing application and the desired integrated sensor circuit 1 geometry. In accordance with some embodiments of the disclosure, the sensing element can include an arrangement of electrodes, e.g., a centrally located reference electrode (e.g., a rectangle of 50 μm by 1500 μm), an outer counter electrode (e.g., a rectangle of 600 μm by 1500 μm), and a working electrode (e.g., a 150 μm by 1500 μm) located between the reference electrode and the counter electrode.

In another embodiment, the sensing element can include 2 working electrodes of 20 μm by 80 μm, 7 counter electrodes of 60 μm by 80 μm, and one reference electrode of 20 μm by 780 μm. In general, it would be acceptable to vary the area of the working electrode from half to double that listed directly previously. In various instances, it would be acceptable to make the working electrode with as little as 15× times smaller area. The counter electrode should be always larger than the working electrode. In various embodiments, the counter can be as little as 3× the area of the working electrode; however, the maximum size of the counter electrode is only limited by the area of the implantable multianalyte monolithic integrated circuit. With respect to the reference electrode, smaller is better. For all practical purposes there is no electrical lower bound on the size of the reference electrode. It needs to be close to each counter. If, however, the reference electrode, is made of a material that may be consumed, such as AgCl, it is advisable for the counter to be of similar size to the working electrode.

### A.2) Multiple Metal Deposition

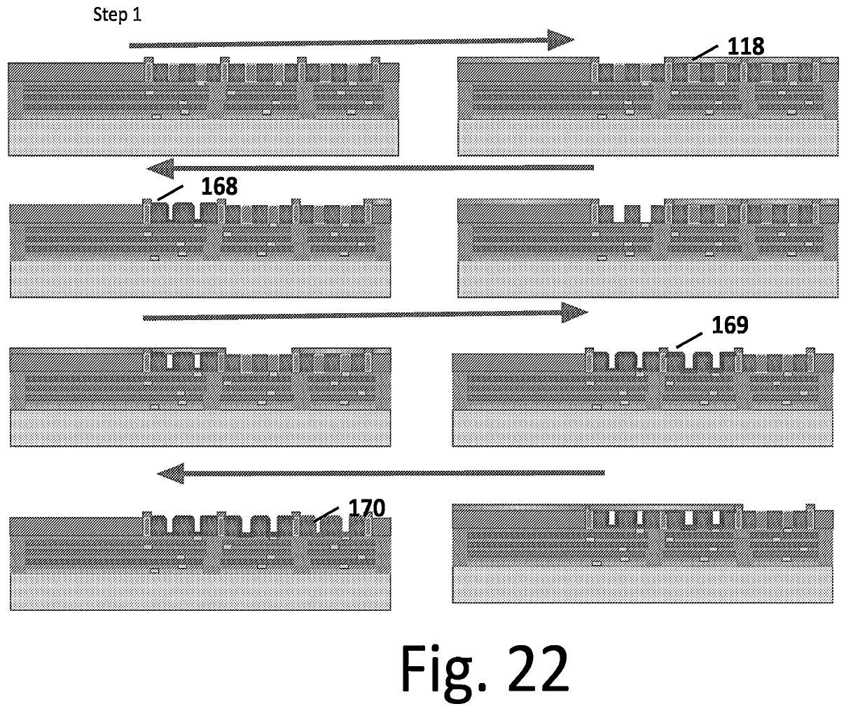

FIG. 22 shows the scheme in which the CMOS precursor has 3 different working electrodes that all need to be coated with different types of electrode material (typically noble metal but can be other types of conductive materials as well). It shows how the Photoresist 118 can be used to create opening on one working electrode while rest are covered with the photoresist, in a lithographic patterning process. Afterwards, either the metal or the insulator in the opening can be etched to make the patterned (waffle) electrodes. After this, a suitable conducting material (typically Platinum or Gold) can be coated on the surface of the device using PVD. In the opening area, the metal would cover the sensing element electrode while it would cover the Photoresist in the rest of the device. Next, lift-off can be used to remove the photoresist which also removes the metal above it, only leaving the metal where there was an opening in the photoresist. This leaves a thin film of first conductor 168 on the CMOS device in select areas on the select electrode(s). The process of lithographically creating an opening, followed by etching to create hollow structures and coating with a conducting element which is then removed from all other places by lift-off and only is left on the next electrode as layer 169 in performed. The process is repeated to have a third electrode coated with a conducting material 170. Between etching the material from one or more electrodes and coating a different material, another lithographic patterning step can be performed to have a cleaner patterning material and/or more selective patterning for the PVD process. The resulting CMOS device with 3 different coatings is shown at the end of FIG. 22. Other than the top metal, the rest of the device remains the same.

B) Wet Chemistry and/or Polymer Deposition

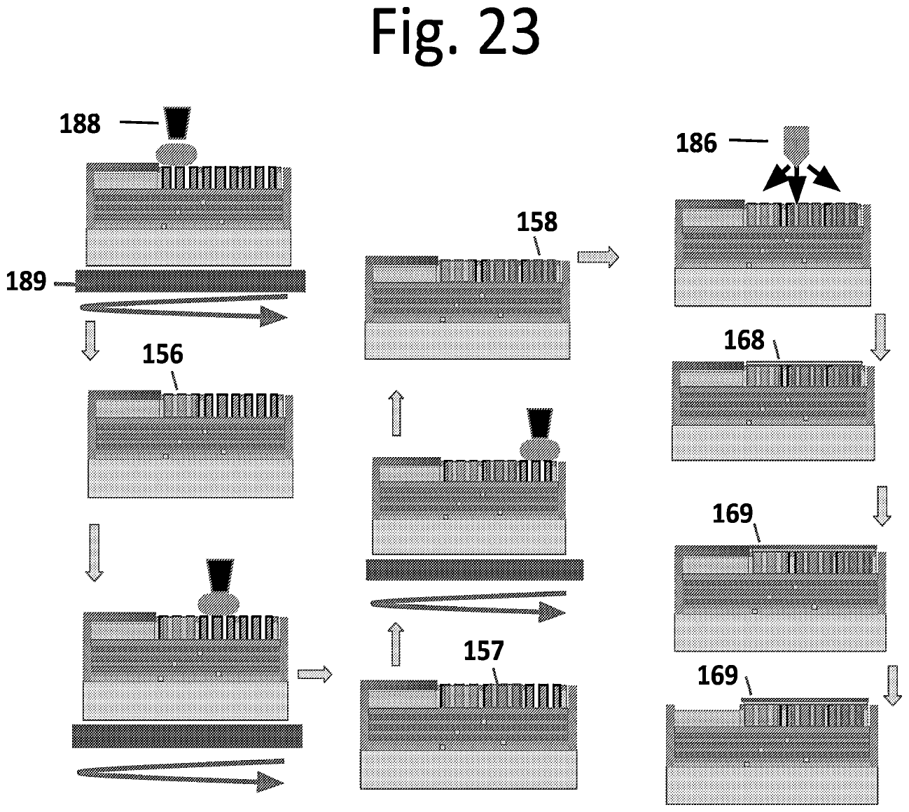

FIG. 23 demonstrates an embodiment of the use of fine droplet/spray deposition systems to precisely cover the integrated sensing element of a precursor with controlled amounts of a functionalization layer. Optionally, the use of a fine droplet/spray deposition system using solenoid and/or piezoelectric controlled actuation-based spray heads 188 simultaneously with spinning on table 189 can improve the uniformity of the functionalization layer (e.g., hydrogel). Spinning is optional and the process can work without spinning. For example, solenoid and/or piezoelectric controlled actuation-based spray heads 188 can be used to deposit, in pico to nano liters, precise amounts of enzyme (e.g., Glucose Oxidase) solutions and crosslinking agents (e.g., Glutaraldehyde) solution droplets to make an enzyme (e.g., glucose oxidase-based) hydrogel on the sensing electrode. The substrate can be spun after the droplet deposition to control the thickness of the hydrogel layer more precisely. A substrate 110, a metal-insulator-metal stack 129, a thin layer of desired material 138, and a top insulator with an optional additional insulator/protection polymer layer 118 remains.

An enzyme hydrogel is an exemplary functionalization layer. As noted above, in one embodiment, an enzyme is immobilized on the sensing element in a hydrogel (e.g., a cross-linked protein hydrogel). This can be done at a thickness 0.01 μm to 50 μm. The enzyme hydrogel layer may be under 3500 nm in thickness. Alternatively, the enzyme hydrogel layer may be less than 1000 nm in thickness. Further alternatively, the enzyme hydrogel layer may be between 200 nm and 800 nm in thickness. Further alternatively, the enzyme hydrogel layer may be between 600 nm and 800 nm in thickness. This can be done using different techniques. As an example, this can be done through immobilization of the enzyme such as GOx (Glucose Oxidase) in a hydrogel created by proteinaceous material with glutaraldehyde as the crosslinking agent. The proteinaceous material can be a blocking agent such Human Serum Albumin (HSA) or Bovine Serum Albumin (BSA) or some other Serum Albumin (SA). Herein a “blocking agent” is a material that blocks unwanted binding interactions of the sensor or sensor components with tissue materials and fluids and avoids or decreases fouling of the sensing element. Glutaraldehyde can be dispensed before application of the remaining elements of the hydrogel. Subsequently, a mixture of GOx, Serum Albumin, and in some embodiments, catalase, can be placed on the precursor. Glutaraldehyde can be used to aid hydrogel formation, and/or catalase can be used to increase sensor longevity by mitigating excess hydrogen peroxide production during glucose sensing. In accordance with some embodiments of the invention, it may be desirable to remove excess hydrogen peroxide from the hydrogel during glucose sensing, so a mixture of Catalase with GOx and Serum Albumin can be used. In accordance with some embodiments of the disclosure, it may be desirable to form the hydrogel after the solution is already dispensed on the electrode, by adding Glutaraldehyde to the mixture after it is dispensed on the electrode, for example, in a separate step. In accordance with some embodiments of the disclosure, Glucose Dehydrogenase can be used as the glucose sensing enzyme, in addition to or instead of Glucose Oxidase.

In some embodiments, the system can be coated with different enzymes on different sensing element electrodes. In FIG. 23, it shows the droplet deposition and optional spin coating process is repeated to form layers 156, 157, and 158 on different sensing element electrodes (e.g., different working electrodes or a combination of different working, counter, and/or reference electrodes).

In accordance with some embodiments of the disclosure, to selectively functionalize the sensor electrodes, a precise deposition of nano- to pico-liter of the hydrogel can be utilized. In one embodiment, the substrate can be heated or cooled and kept at a controlled temperature (e.g., 25 degrees Celsius to 35 degrees Celsius, with 25 degrees Celsius being an embodiment) in a controlled environmental chamber (e.g., to control temperature, humidity, chemical composition of the environment). Then, an accurate dispensing instrument (such as a BioJet Elite on a AD6020 aspirate dispense system by Biodot, Irvine, CA) with precise x, y, and z position control can be utilized. In one embodiment, the sprayed solutions are a protein solution of GOx and/or Catalase and HSA (1200 mg, 12 mg, and 1000 mg respectively in 15 ml DPBS, Sigma Aldrich Product codes G2133, SRE0041, SRP6182, D8537) and a crosslinking agent solution of 1% w/w glutaraldehyde in DPBS (Sigma Aldrich, St. Louis, MO, product codes G5882, and D8537).

In accordance with some embodiments, deposition can be performed in three steps to achieve a hydrogel of repeatable and controlled hardness and composition: 1) dispensing glutaraldehyde, 2) dispensing the mixture of GOx and SA, 3) dispensing glutaraldehyde. The three deposition steps can be done almost simultaneously through the use of three dispensing nozzles as gel formation starts happening almost instantaneously once SA and glutaraldehyde come to contact. In a different method, glutaraldehyde is only dispensed once. With the three-step process, or with a process where only steps 1 and 2 are performed, controlled temperature (e.g., 25 degrees Celsius) of the sensing element electrode surface and controlled environment (e.g., 80% RH, low particle count in air) during and after dispensing helps with uniform gel formation.

In accordance with a different embodiment of the disclosure, spin coating and/or spray coating can be used to achieve functionalization by applying the sensing chemistry on the sensing elements, instead of precise deposition. In this method, enzyme hydrogel mixture is dispensed or sprayed on the precursor or even entire wafer using nano-droplet dispenser, spray head, or pipette. The hydrogel formulation can be the same as that used in precise deposition. The wafer is then spun to achieve a thin sensing layer at controlled speed (between 200 to 20000 rpm with 2000 rpm being an embodiment) for set time (10 seconds to 3 minutes, another embodiment being 1 minute) to achieve a thin (10-50000 nanometer thick, e.g., 2-6 micrometer thickness) layer sensing chemistry.

In accordance with a further alternative embodiment, instead of precise deposition or spin coating and/or spray coating, the functionalization layer (e.g., hydrogel: crosslinking agent and or the protein mixtures) can be deposited on the wafer via dipping. In some instances, the sensor chips or the entire wafer can be mounted on a substrate that can be dipped vertically or horizontally in a solution of enzyme or enzymes and serum albumin and optionally glutaraldehyde. The hydrogel formulation can be the same as that used in precise deposition. The substrate can be dipped and dried one or more times for a total processing time ranging from 2 minutes to 2 hours depending on desired gel thickness and consistency. In some embodiments, the sensors can be dipped for one minute and dried in a chamber with 80% relative humidity for 5 minutes for 10 cycles for a total processing time of 60 minutes. In accordance with some embodiments, the sensors can be dipped in protein solutions and glutaraldehyde solution, sequentially. For instance, if there are a variety of sensing chemistries dispensed on the sensor, and many of these produce hydrogen peroxide, then, after the coating(s) are dispensed, the whole wafer can be dip coated in catalase solution followed by dip coating in glutaraldehyde to immobilize the catalase on the sensors' surface. In some embodiments of the disclosure, a cleaning solution such as DPBS can be used between dipping steps in order to prevent beading of the solutions on the sensors and resulting loss of uniformity.

In some cases, the hydrogel can conform to the pattern of underlying pillars to resultin patterned layers. Such patterning can allow for faster response time (lesser delay) but may have a shorter lifetime compared to sensors with thicker hydrogels and polymer layers. The hydrogel and polymer can be shaped either like blocks of materials covering the pillars or shaped liked pillars with empty space in between the pillars. The different shapes can be obtained by process control (e.g., if droplet functionalization of hydrogel is used a block of hydrogel formed; however, if spray, a thin layer mostly sticks to pillars and provides a conformal coating). In accordance with some embodiments of the disclosure, each working electrode can be isolated from the rest of the working electrodes and allow for unique functionalization of individual electrodes, e.g., different electrode sensing chemistries. These methods for selective functionalization of individual working electrodes are in addition to the ability of precise deposition to achieve this result as noted above. Options to facilitate selective functionalization include stencils, lithographic patterning, nanoimprint lithography, and selective activation. In accordance with some embodiments of the disclosure, where isolation is required, all the sensing element components for any one analyte application can be dedicated (e.g., separate working, reference and counter electrodes) and isolated from others.

Stencils can be used to selectively functionalize sensing elements with different chemistries. In these embodiments, a stencil, e.g., a metal sheet with holes corresponding to sensing element surfaces, can be placed on the die or wafer. Then sensing chemistries can be dispensed, dropped, dipped, or sprayed, or otherwise deposited. In some embodiments spraying is used. Then the stencil can be lifted from the surface to leave defined sensing chemistries deposited on sensors. The stencil process can be repeated or combined with other processes to achieve a variety of chemistries.

Alternatively, wafer scale lithographic patterning can be used. In some of these embodiments, a light-active chemical (e.g., a photoresist) can be placed on the die or wafer and patterned using light and a developer as known to those skilled in the art. Then dispensing, spin or spray coating, dipping, or any method described in the above surface functionalization paragraphs herein can be employed to deposit sensor chemistries on the specific sensors.

Nanoimprint lithography is yet another technique that can be used for this purpose. In this case, a special printing head/stamp can be used to transfer small gels on to the sensing element surfaces (e.g., electrode surfaces). The gel is first formed on this stamp (which can be made using lithographic patterning or molding) using any of the methods discussed herein (e.g., nano-droplet dispensing, spin coating, spray coating, dipping). Then the stamp is placed on the desired wafer and a method is used to release the hydrogel to the specific sensors on the wafer. This is facilitated either by increasing gel adhesion with the sensors on the wafer (e.g., by surface activation of sensors and particularly surfaces of sensing elements in a manner such as with oxygen argon or air plasma) or by using heat/UV to create some change on the stamp which releases the gel.

Specific sensing elements can also be patterned by selectively activating the sensing element surfaces (e.g., with an oxygen, argon, or air plasma, or chemical modification) and sensing chemistries can be deposited using any of the methods discussed herein (e.g., nano-droplet dispensing, spin coating, spray coating, dipping). Then, the sensing chemistries can be removed (e.g., washed with deionized water, or a mixture of deionized water and detergent such as 10% (w/w) Extran (MilliporeSigma, Burlington, MA) in deionized water) such that only sensing chemistries bonded to the activated surfaces remain.

FIG. 23 step 2 shows an embodiment of a device after deposition of the functionalization layer 156 using solenoid and/or piezoelectric controlled actuation-based spray heads 188. A substrate 110, a metal-insulator-metal stack 129, a thin layer of desired material 138, and a top insulator with an optional additional insulator/protection polymer layer 118 remains. The process is repeated for other sensing element electrodes with different solutions to result in different hydrogels on different electrodes.

FIG. 23 step 7 shows an embodiment of the use of spray coating (e.g., using fine dispensing heads 186) to make a film (stack of one or more thin films) on sensor surface to coat the functionalization layer, using an appropriate spray head 186 which showers microdroplets on an area of the sensing element. A substrate 110, a metal-insulator-metal stack 129, a thin layer of desired material 138, a first functionalization layer 156, a second functionalization layer 157, a third functionalization layer 158, and a top insulator with an optional additional insulator/protection polymer layer 118 remains.

In an optional embodiment, a crosslinking and/or drying step can be done in a chamber saturated with a crosslinking agent vapor, e.g., glutaraldehyde vapor, to aid or obviate the need for crosslinking via crosslinking agent via further application. For example, for vapor crosslinking a crosslinking agent in the solution may not be required. In accordance with some embodiments of the disclosure, the protein solutions can be precisely deposited (using a precision instrument as described above) on the sensor electrodes and spread using spinning in the presence of crosslinking agent vapor.

Before and/or after functionalization, (but typically after functionalization) different films (e.g., membrane materials) can be used to protect and/or restrain the functionalization materials on the sensing element 160 and achieve a desirable signal response for a particular sensor configuration. In some embodiments of the disclosure, a diffusion limiting layer can be useful.

For example, in the body there is 30 to 300 times more Glucose than Oxygen. If the sensing mechanism has a 1:1 stoichiometry (e.g., Glucose detection using GOx uses 1 molecule of Oxygen for every molecule of Glucose), then the sensor placed without a limiting membrane will be limited by oxygen concentration and will not be able to sense glucose for the entire physiological concentration (e.g., 40-400 mg/dl). A polymer membrane can be deposited to act as a suitable diffusion barrier that allows oxygen to go through unhindered but hinders glucose diffusion.

Examples of workable polymer membrane materials include polyurethane, a mixture of polyurethane and silicone, as well as a mixture of polyurethane and PEG. In accordance with some embodiments, the thickness of the polymer membrane can be in the range from 0.1 micron to 15 microns. The polymer membrane may be between 200 nm and 10,500 nm thick. Alternatively, the polymer membrane may be between 200 nm and 1500 nm thick.

Adhesion between the membrane coating and the underlying hydrogel, or between layers of coating, can be facilitated by use of chemicals (e.g., silanes, aldehydes) and/or physical processes (e.g., corona treatment, oxygen plasma, gas plasma, mechanical roughening). Specific membrane materials and construction can be used to further improve sensor performance. In one embodiment of the disclosure, a combination composition of polyurethane and silicone can act as a filter to regulate diffusion of glucose and as an oxygen recycling membrane as well as providing a biocompatible material. Oxygen recycling can improve the efficacy of the sensor, while the biocompatibility can allow the sensor to work for longer. To cover the sensor uniformly and minimize sensor to sensor and batch to batch variation, membranes can be deposited on the sensor through spotting (droplet coating), spraying or through wafer-level spin coating. Membranes can also be deposited on the backside of the wafer to increase biocompatibility. Another method to uniformly deposit membranes is to employ spray coating with a special instrument utilizing overlap between multiple depositions to achieve a uniform overall thickness.

A specific workable polyurethane membrane coating process includes loading 1% PurSil from DSM in THF (DSM Biomedical, Exton, PA and Sigma Aldrich, St. Louis MO) into an Air-jet spray coating unit (BioDot, Irvine, CA). A single coat of 1.25 microliter/cm is applied at 9 PSI pressure on sensor area, with dispensing height and aperture optimized for each coating unit installation. The wafer is dried in a vacuum oven at 35 degrees Celsius and 25.6 mm-Hg pressure for an hour and in ambient conditions for at least 12 hours (overnight). A second coat is applied, and sensors are dried with the same parameters. The sensors are allowed to stabilize in PBS (Sigma Aldrich, St. Louis MO) for 72 hours and characterized for analyte response.

Optionally, a membrane coating can also be patterned to reduce cell attachment. This patterning can be done using oxygen plasma or using nanoimprint lithography (bio-stamping). For oxygen plasma, after the membrane is coated on the surface, it is exposed to a high-power oxygen plasma (e.g., 300 Watt) in a plasma chamber without any mask or with a lithographic mask (e.g., AZ5214E for Photolithography, PMMA for electron beam lithography) or a stencil. The oxygen plasma will etch the exposed material and the material under the etch mask will be protected, hence shaping the surface. For stamping, the membrane is coated and patterned while being on a different substrate (e.g., Silicon substrate). Next, the membrane and the sensor surfaces are exposed to some process that prepares their surface for strong adhesion. One example is to expose both materials to a short, low-power oxygen plasma dose or to chemical linkers like Xylene. Next, the membrane is placed on the sensor surface and the bonding process is allowed to continue for some time. At the end, the substrate is gently removed, and the patterned membrane remains on the sensor surface.

FIG. 23 step 8 shows an embodiment of a device after deposition of a second film 168, such as a interference rejection layers, immune response suppressing layer, and/or biocompatibility layer, on the sensing element. A substrate 110, a metal-insulator-metal stack 129, a thin layer of desired material 138, functionalization layers 156, 157, 158 and a top insulator with an optional additional insulator/protection polymer layer 118 remains.

Another example of polymer coating, other than a membrane, is use of interference rejection layers that can be coated on the electrodes before or after surface functionalization. These layers can similarly be coated using spraying, dip coating, electrochemical coating, spin and/or spray coating. In accordance with some embodiments of the invention, a coating including o-phenylenediamine can be used for rejecting Ascorbic acid and/or Acetaminophen in glucose sensing applications.

Another example of polymer coating which can be used includes immune response suppressing layers. Implanted sensors can be attacked by the foreign body or immune response of the body. This can be mitigated by incorporating coatings that inhibit response and/or mitigate the effects and decrease this foreign body response. Drugs such as dexamethasone or nitric oxide limit such response. Note that, in some embodiments of the disclosure, drugs that inhibit adverse response by the body (e.g., dexamethasone, nitric oxide) can be mixed, encapsulated, or chemically included in the functionalization layers and/or membrane layers, instead of a separate coating, in a way that allows slow release of the drugs throughout the functional lifetime of the sensor. Another example of polymer coating which can be used includes a biocompatibility layer.

To improve biocompatibility of the system, the sensor can be coated with a biocompatible material. Proteins attach to hydrophobic surfaces; thus, an option to improve biocompatibility is to cover the device with a hydrophilic or super-hydrophilic polymer (Note that polyurethane, an option for membrane formation, is hydrophobic). For example, the biocompatible material can be poly-HEMA. A layer of pHEMA can be formed with a thickness of 5 microns to 100 microns and a preferable thickness of about 10 microns. In some cases, a copolymer of a biocompatible material can be made with polyurethane to coat the device in a single step.

An additional/alternative biocompatibility layer can be the deposition of Titanium and/or Platinum or a catalase hydrogel to mitigate the effects of reactive oxygen species. For example, a layer of catalase can be coated over top a layer of GOx hydrogel. The layer of catalase can be, for example 0.05 pin to 25 pin, alternatively 0.1 μm to 3 μm. It has been shown that reactive oxygen species from glucose oxidase can damage surrounding tissue. Platinum is known to breakdown the reactive species into less corrosive byproducts and hence is ideal for this application. For instance, platinum microspheres can be dispersed within the pHEMA hydrogel or any other hydrogel and used to coat the surface. In short, a solution of platinum spheres in water can be used to make the hydrogel and ultrasonic mixing of the components can be used to ensure proper dispersion of the spheres throughout the hydrogel. In a further embodiment of the disclosure both a hydrophilic biocompatibility layer and a means to quench reactive oxygen species are used in combination with an immune response limiting element. For example, dexamethasone (0.01%-3% w/w) can be mixed into the polyurethane layer. Poly-HEMA layer can be patterned with nanoimprinting to achieve a super-hydrophilic surface. 2 nm thick Ti/2 nm thick Pt can then be sputtered on the surface to quench reactive oxygen species while maintaining superhydrophilicity and porosity. As noted above, multiple layers of membrane and/or polymer materials can be applied to the implantable monolithic sensing circuit.

Another example of biocompatible layer (also known as sensor-tissue interface layer) is PVA. A specific workable PVA includes the following: Poly (vinyl alcohol) (MW 89,00-98,000 99%+ Hydrolyzed, Sigma 341584) was mixed with DI water 4% (w/v) (1-10% acceptable), by slowly adding PVA into DI water heated to 80 C and stirred at 400 rpm with a stir bar. The solution was capped and let stir for 12 hours prior to use.

To make the PVA layer, the solution is drop casted or spin coated on the sensor surface. In a typical recipe, the solution is dispensed to cover more than 90% of the surface to be coated (e.g., wafer, die, or flex PCB with the sensors attached). The surface is then spun, first at a lower speed (e.g., 500 rpm) for 10 seconds to spread the solution and then accelerated to a higher speed (e.g., 500-5000 rpm with 3000 rpm as a typical case) to get to a desired thickness. The sensors are then left to sit in a controlled environment (e.g., an oven set at 35° C.) for a known time (e.g., 24 hours) for the PVA gel to form.

In some embodiments, the multi-analyte sensing chip is coated with the biocompatible layer after being coated with the enzyme and optionally the diffusion-limiting membrane. In some cases, the chemistry is protected with a protecting layer (e.g., a photoresist or a sacrificial layer) before the next processing steps. FIG. 23 step 9 shows the device being coated with a protective layer 169. The protective layer can be patterned and removed from the bonding pad 170 to enable device packaging afterwards.

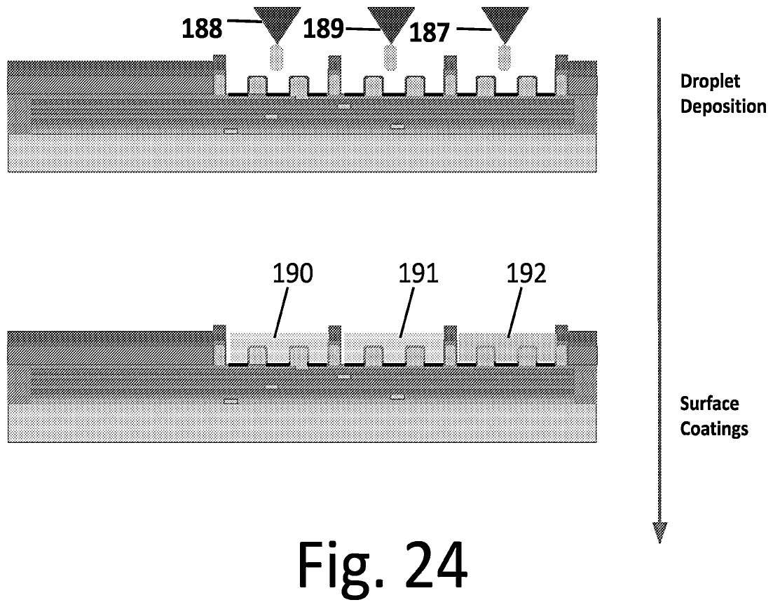

FIG. 24 shows a scheme of functionalizing three different sensing element electrodes with 3 different solutions to form three different layers 191, 192, and 190 on the CMOS device. This scheme does not use spinning. The 3 solutions can be deposited using three different deposition heads 187,188, and 189 on the same deposition system (e.g., Biodot Ultra and Rainmaker systems). The materials 191,192, and 190 can be same as materials 156, 157, and 158 or different.

B.1) Sacrificial Layer Wet Chemistry and/or Polymer Deposition

In some embodiments, lithographic patterning can be used to pattern one or more types of surface coatings on the CMOS device. Such patterning involves using lithographically patternable materials (e.g., photoresists, electron beam resists) that can be used to create selective structures on the CMOS device using light and/or electron beams or other such methods. These materials can be used to pattern the surface coatings directly or through an intermediate layer called a sacrificial layer. The sacrificial layer can be a layer more suitable for pattern transfer and more favorable to the surface coating materials than the lithographically patternable materials themselves. For example, in some embodiments, a Polyacrylic acid (PAA) layer can be used as a sacrificial layer since it is more easily removable than the lithographically patternable materials themselves and is more compatible with hydrogel materials.

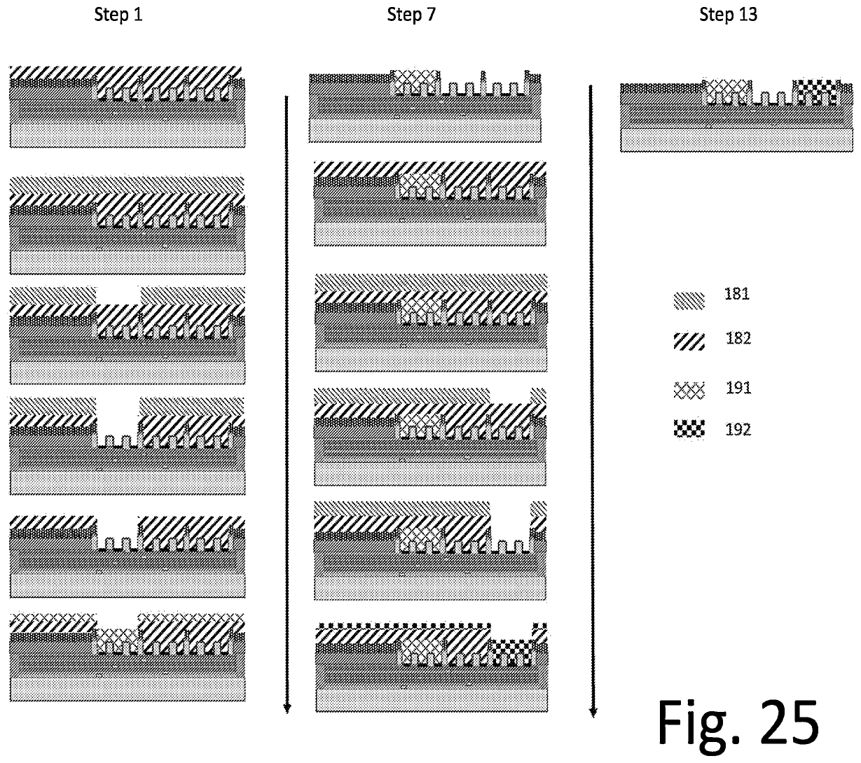

FIG. 25 shows one scheme of using spin-coating and/or spray coating to pattern three coatings uniformly across three electrodes on the sensing platform utilizing a sacrificial layer. The chemistries can be deposited in fewer or more steps depending on what steps each chemistry can tolerate, and on the treatments needed for each chemistry (e.g., plasma treatment, silane treatment).

FIG. 25 starts with a post processed sensor chip, i.e., wafer with conductive material deposited upon sensing electrodes as seen in the last step of FIG. 21 and FIG. 22.

In FIG. 25 Step 1, a sacrificial layer 182 is deposited on the sensor (e.g., by spin coating, spray coating). In some embodiments a water-soluble sacrificial layer can be used. For example, a mixture of Polyacrylic acid can be used to make a sacrificial layer. Same can be done by using Polyvinyl alcohol or other water-soluble materials as well as material that can be dissolved in other solvents. In some embodiments (not pictured) water-soluble sacrificial layers can be used to protect the sensing chemistries to allow for patterning the sensing chemistries with a photoresist via a lift-off process, where the water-soluble layer would protect the enzyme layer from the photoresist and chemical and physical methods used to pattern and clean the photoresist, including but not limited to developer, acetone, or plasma etching.

In FIG. 25 Step 2, a patterning chemistry 181 (e.g., photoresist, e-beam resist) is deposited over the sacrificial layer 182 (e.g., by spin coating, spray coating).

In FIG. 25 Step 3, the patterning chemistry 181 (e.g., photoresist, e-beam resist) is patterned.

In FIG. 25 Step 4, after patterning of the patterning chemistry 181 in Step 3, the sacrificial layer 182 is dissolved in a manner as to correspond to the pattern created by the patterning chemistry. This is called the pattern transfer process.

In FIG. 25 Step 5, the patterning chemistry 181 is completely removed. This can be done using different methods like using a chemical (e.g., photoresist remover solvents like acetone) or dry plasma or a combination of these methods based upon the type of material and the compatibility with the other materials on the CMOS device.

In FIG. 25 Step 6, chemistry 191 is deposited (e.g., by spin coating).

In FIG. 25 Step 7, the sacrificial layer 182 is removed (e.g., by dissolving in water in case of PAA) which also removes the chemistry 191 in areas where it was resting on the layer 182. This leaves the chemistry 191 on only the areas where layer 182 has been previously removed by patterning.

From FIG. 25 step 8 onwards, the process is repeated to form patterned sacrificial layer and transfer the pattern of chemistry layers 192 and 193 on the CMOS device.

In FIG. 25 Step 8, sacrificial layer 182 is deposited again.

In FIG. 25 Step 9, The patterning layer 181 is deposited again.

In FIG. 25 Step 10, the patterning layer 181 is patterned using the desired pattern for next chemistry layer.

In FIG. 25 Step 11, the pattern from second layer 181 is transferred to second layer 182.

In FIG. 25 Step 12, the second patterning layer is totally removed and the chemistry layer 192 is deposited (e.g., by spin coating). The two steps are shown together for brevity but are done the same way as for layer 191.

In FIG. 25, Step 13 the sacrificial layer 182 is dissolved leaving the target chemistries 191, and 192 on the target electrodes.

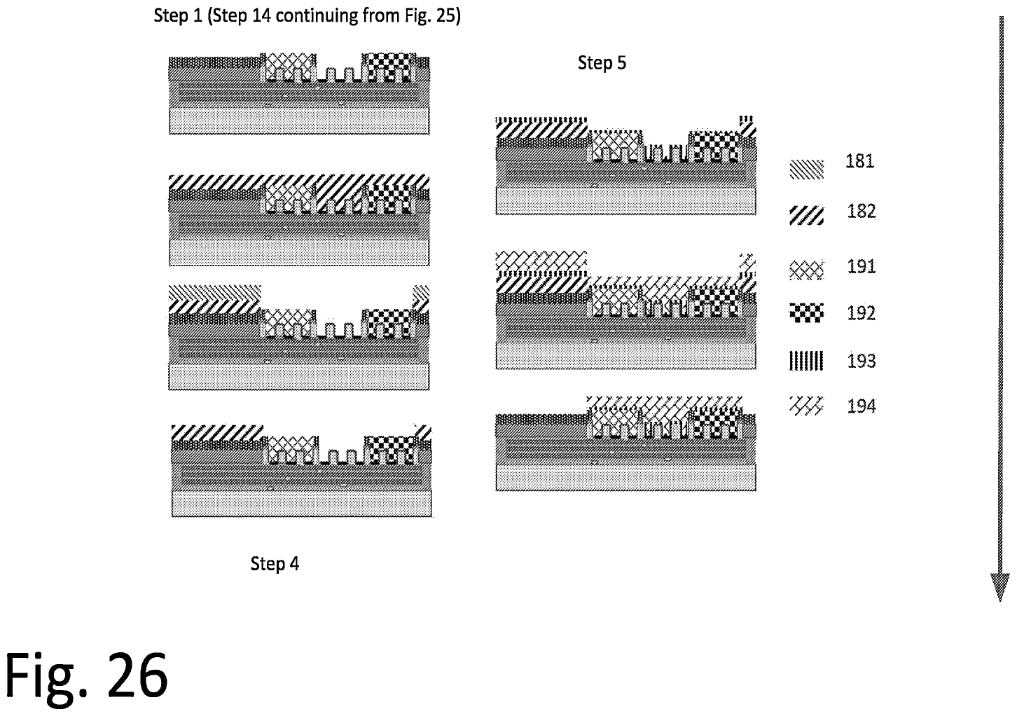

FIG. 26 shows a continuation of the process shown in FIG. 25 to coat the electrodes on the CMOS device with a membrane.

FIG. 26 Step 1 shows the starting device is same as the ending device of FIG. 25.

FIG. 26 Step 2 shows the device is coated with a third sacrificial layer 182.

FIG. 26 Step 3 shows the third sacrificial layer 182 is patterned (e.g., using lithographic patterning) to create an opening on the three sensing element electrodes while covering the rest of the CMOS device.

FIG. 26 Step 4 shows the photoresist is removed to leave the patterned sacrificial layer on the CMOS device.

FIG. 26 Step 5 shows the CMOS device is coated with a membrane 193 (e.g., using spin, dpi, spray coating etc.)

FIG. 26 Step 6 shows the CMOS device is coated with a biocompatible coating 194 (e.g., using spin, dip, spray coating etc.)

FIG. 26 Step 7 shows the sacrificial layer is removed to leave the CMOS device coated with the two chemistry layers 191 and 192, with a membrane coating 193, and with a biocompatible coating 194.

This process can be repeated for any number of chemistry layers to selectively coat the CMOS device with different chemistry layers.

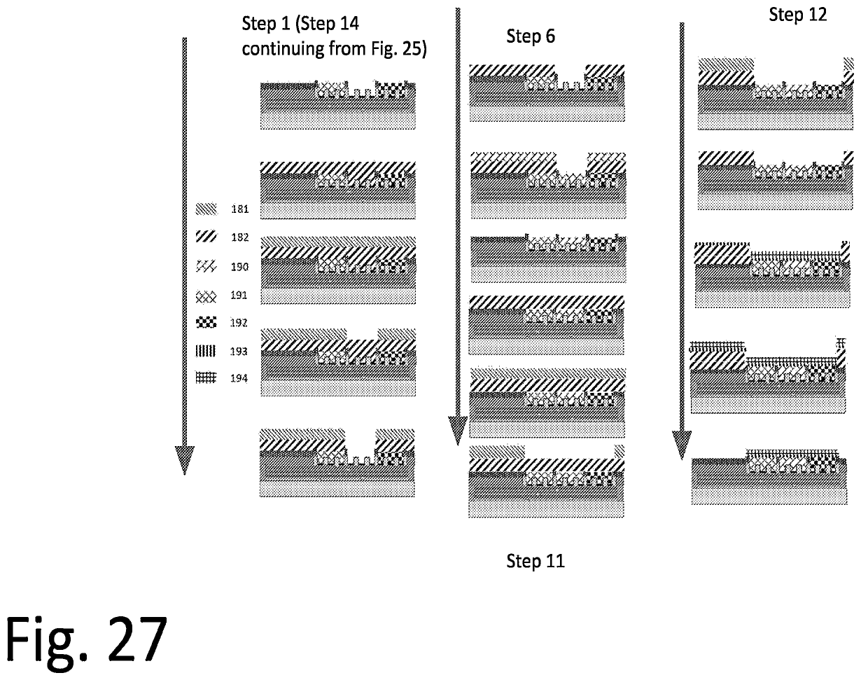

FIG. 27 shows one scheme of using spin-coating to pattern 3 different (as an example) coatings on three different electrodes on the sensing platform. FIG. 27, similar to FIG. 26, is a continuation of FIG. 25. The chemistries can be deposited in fewer or more steps depending on what steps each chemistry can tolerate, and on treatments needed for each chemistry (e.g., plasma treatment, silane treatment).

In this example in FIG. 27 step 1, shows the device at the end of FIG. 25. In FIG. 27 Step 2, a sacrificial layer (e.g., poly acrylic acid) 182 is deposited on the sensor (e.g., by spin coating, spray coating). In Step 3, a patterning chemistry (e.g., photoresist or PR, e-beam resist) is deposited over sacrificial layer (e.g., by spin coating, spray coating). In FIG. 27 Step 4, the PR is patterned. After deposition and patterning of the PR, the sacrificial layer is dissolved in photoresist developer as shown in FIG. 27 step 5. After that, the PR can be removed as shown in FIG. 27 step 6 and a third chemistry layer 190 is coated on the device as shown in FIG. 27 step 7. Afterwards, the SL is dissolved which lifts off the chemistry from undesired areas as shown in FIG. 27 Step 8.

After coating with the three chemistries, the sensing electrodes can be further coated with other layers to improve sensor functionality. For example, the sensor can be coated with one or more polymer membranes (layer 193). These layers can be patterned using a similar process, with or without a sacrificial layer. An example of using sacrificial layer to create opening for the membrane 193 over all 3 working electrodes is shown in FIG. 27, steps 9-12 (similar to sacrificial layer patterning in the previous steps). The same pattern can be used to coat the device with a biocompatible coating 194. The sacrificial layer is then removed. The resulting device with the 3 chemistry layers (191,192,190), the membrane 193, and the biocompatible coating 194 is shown at the last step of FIG. 27.

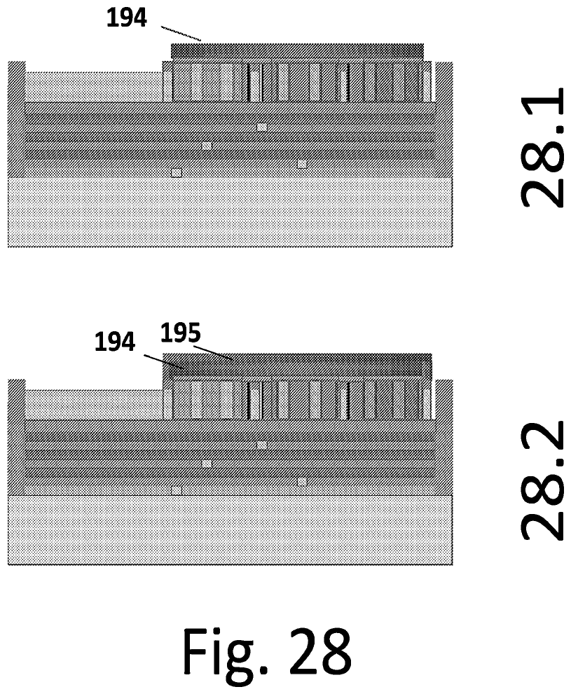

After coating with the desired surface chemistry layers, the CMOS devices can be coated with one or more protection layers to protect the sensor surface and/or the chemistry during further processing. The CMOS device with the surface chemistry without the protection layer is shown in FIG. 28A and with the protection layer 195 is shown in FIG. 28B.

C) Finalization

After deposition of one or more membrane and/or polymer materials, in an embodiment, the implantable monolithic sensing circuit can be considered complete.

In accordance with some embodiments of the disclosure, a post-processed sensor wafer can be cleaned with deionized water and/or pressurized gas and dried in vacuum oven (20-400 degrees Celsius, e.g., 40-200 Celsius; 0 to 30 mm-Hg below atmosphere, e.g., 26 mm-Hg). In accordance with some embodiments of the disclosure, a cleaning and drying step can be followed by a plasma cleaning and surface activation step. In some embodiments, the sensor can be cleaned under 50-600 mTorr pressure of oxygen or air or argon plasma with a power of 50-400 W. In some embodiments, Oxygen plasma at 100-500 mTorr, with a power of 90-200 W can be used. In accordance with some embodiments of the disclosure, after post-processing and drying, the wafers or sensors can be placed in a humidity controlled nano/pico liter dispenser equipped with aluminum chilled plate calibrated to be able to operate at 80% RH and 25 degrees Celsius plate temperature. The dispensation is used to deposit controller amounts (e.g., pico liters) of functionalization solution (e.g., GOx; HSA, LOx:HSA followed by Glutaraldehyde solution) to form surface coatings to make the CMOS device sensitive and selective to different analytes.

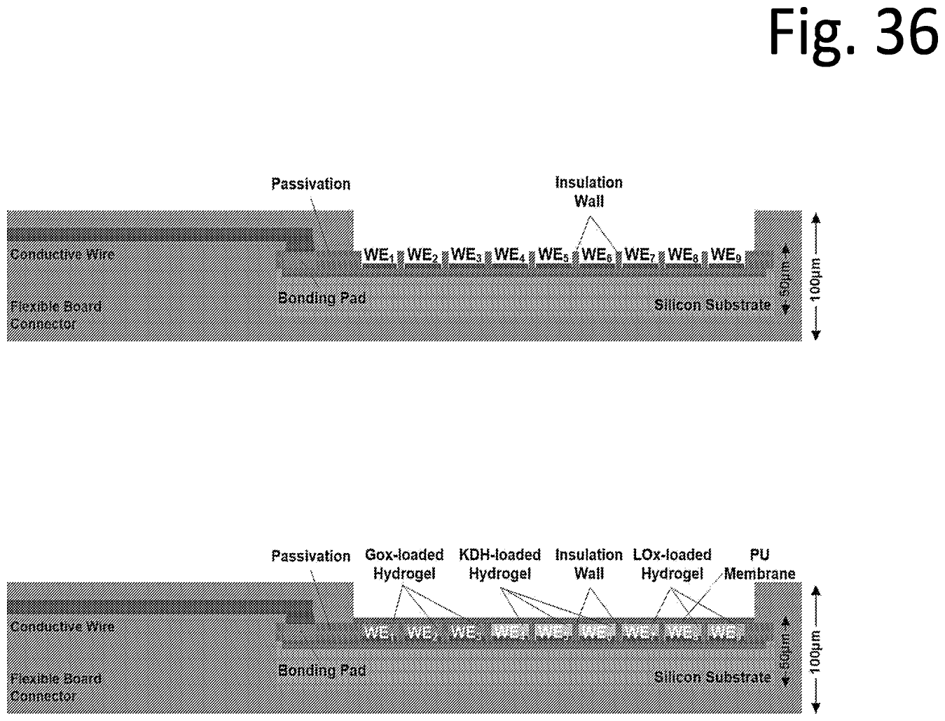

In some embodiments, the CMOS integrated sensing circuit 1 is attached to a flexible connector 3 to form a packaged device. FIG. 36A shows the components of an embodiment of a completely processed implantable multianalyte monolithic integrated sensor circuit including the silicon substrate with integrated contact pad 170 attached to flexible connector 3, the integrated working electrodes, WE₁, WE₂, WE₃, . . . , WE₉ representing the integrated sensing element, said working electrodes separated by insulation walls 118. The other electrodes (e.g., the counter and reference electrodes are present on the same chip but are not visible in this side view).

FIG. 36B shows the working electrodes coated with the functional chemistry, for example Gox-loaded hydrogel 191, Lactate Oxidase (L)x) loaded hydrogel 192, and Ketones Dehydrogenase (KDH) loaded hydrogel 190 and the polymer coating example PU membrane 193.

To get from the CMOS devices to the packaged sensors shown in FIG. 36, there are different manufacturing schemes that can be used. The next few sections explain a few embodiments of the whole device as well as the manufacturing schemes.

### Flexible Connector or Flexible Connecting Wire

In the present disclosure, a physical intermediate transdermal component 3 is used to connect the external transmitter 2 located on the skin to the multianalyte monolithic integrated sensor circuit 1 in the analyte concentration measurement system.

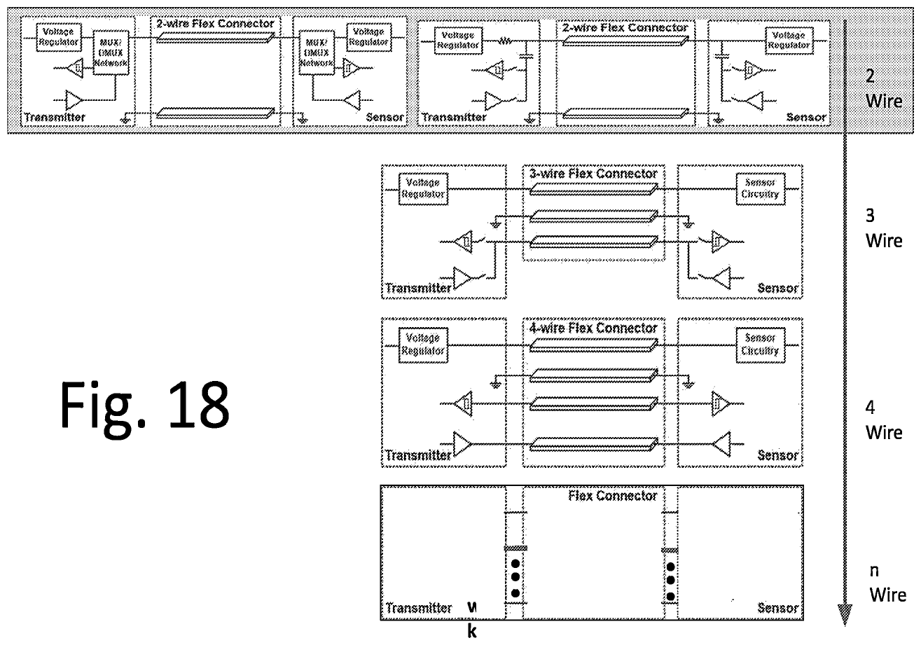

One typical embodiment of the intermediate component is a bidirectional wire connector (flexible connector 3, wire 3, or connector 3). The connector can be a single wire, two-wire, three-wire, four-wire, or higher instance flexible connector 3 as shown in FIG. 18. In some embodiments, the connector can be a flexible printed circuit board (Flex PCB) with the wires being conductive trace(s) therein.

Wires/conductive trace(s) can be made of copper, aluminum, gold, or other conductive material. The connector 3 can comprise a biocompatible polymer such as Parylene (e.g., parylene-C), liquid crystal polymers (LCP), or polyimide, which may almost completely cover any wire/conductive trace(s) except for points necessary to connect to the external transmitter 2 or integrated sensor circuit 1. Some materials are most suitable for others. For example, inert metal conductors (e.g., gold conductors) are better than Copper (Cu) in terms of their stability and biocompatibility. Moreover, LCP inert layers are better than Polyimide inert layers due to their lower water absorption properties.

FIG. 18 shows several schemes to connect the integrated sensor circuit 1 with the transmitter 2 via a flexible connector 3 comprising varying the numbers of wires/conductive traces. The connector 3 can have 1 or multiple wires/conductive traces to flow power and data between the sensor 1 and the transmitter 2.

Data communication can be performed using a two-wire connector per “communication over power” through superimposition of the data signal over the power wire. The same conductors can be used for both data and power (e.g., to save space) as shown in the 2-wire configurations shown at the top of FIG. 18. One of the wires can act as the power supply and signal and the other as the ground. If data and power share a conducting line, a coupling-decoupling (Co-De) network (also termed as Multiplexing-Demultiplexing or MUX/DEMUX network) on both sides of the same conductor is to be used. The Co-De network can be designed to separate data from power on either or both sides.

With regard to MUX/DEMUX network, at the transmitting side, this can be done through an AC coupling capacitor and a tri-state driver in one particular embodiment. At the receiving end, an AC decoupling capacitor can be utilized to extract data by decoupling it from the power signal followed by a hysteresis comparator which detects the signal while being resilient to the signal noise. Since both transmitter and the multianalyte monolithic integrated circuit will be operating in both transmitting and receiving modes, the aforementioned circuitries can be incorporated in both the transmitter and the sensor. Since the data is modulated over the power signal, the power signal received at the sensor is not clean. Hence a voltage regulator is utilized at the sensor to create a clean and stable DC supply. This DC power can be sent to the potentiostat which powers up the integrated sensing element. If a read command is received from the transmitter, the monolithic sensor circuit can send digital readings via the two-wire flexible connector to the transmitter. In some cases, a resistive coupling network can also be used for the MUX/DEMUX.

However, different conductors for data and power can also be used to simplify the system design as shown in the 3-wire configuration of FIG. 18. If full-duplex communication is desired, separate conductors can be extended to use n number of conductors, e.g., for sending redox voltage from the transmitter to the sensor, for sending data for multiple sensors from the sensor to the transmitter and so on.

The flexible connector can fit in a 12 gauge to 32-gauge needle with 23 to 28 gauge being an alternative range and 26 gauge being a further alternative. Rectangular needles can be used instead of standard hypodermic needles, in which case an equivalent needle gauge description can be used as the standard needle gauges are defined for cylindrical tubes and the needles made from those tubes. The equivalent needle gauge can be defined by comparing the hypotenuse of the rectangular needle (based upon the triangle formed by the base and the wall height) with the diameter of the cylindrical needle.

Regarding the mechanical properties of the connector 3, said properties of the connector 3 can be controlled by controlling its conductive materials, insulating layers, and shape. It is important to manage the mechanical properties of the connector to provide a connector of sufficient rigidity and strength to be successfully applied by an applicator yet flexible enough as to not induce a strong immune response by damaging surrounding tissue during user movement.

For example, stiffeners (e.g., polyimide, glass, steel, FR4) can be attached as a bottom layer of the flexible connector 3 to provide additional rigidity.

Alternatively, a layer of bottom Cu can be used to increase the stiffness of the connector 3. This use of standard flexible PCB insulated copper traces to control the stiffness of the flexible connector is a unique feature of this design. Control on size and thickness (e.g., 0.25 Oz, 0.5 Oz, 10z, 2 Oz etc. of Cu) can provide a control on the mechanical properties of the flexible PCB panel from which the flexible connector 3 is cut out later. For example, a panel with a dummy bottom metal underneath the top metal designs can be stronger than a panel without the bottom Cu. The Cu is completely covered with sealed insulators to prevent any interaction with the body fluids. In some cases, Cu can be covered with more biocompatible materials (e.g., noble metals like Palladium and Gold) or can even be replaced by those. This stiffness control enables thinner devices as compared to the use of stiffeners, and hence enables the use of smaller needle sizes which reduces insertion pain and foreign body response. The top metal and the bottom metal layers may also be referred to as “first metal layer” and “second metal layer”, respectively.

Regarding a connector (e.g., flex-PCB), the total thickness in some embodiments can be 100 to 250 μm, preferably 150 μm. A flex-PCB can be made of three layers (1 conductive layer and two insulating layers), five layers, or more depending on the design of the conductive traces and stiffness requirements.

In a specific exemplary embodiment, the flexible connector 3 is a flexible PCB. The flexible PCB is of five total layers (generally known as 2-layer PCB based upon 2 conductive layers) with a top Cu layer of ½ oz (18 μm) and bottom Cu layer of 2 oz (70 μm). A top overlay can be used of 1.5 mils (37 μm)−1 mil adhesive+0.5 mils. The substrate of the PCB can be for example 1 mil (25 μm). Preferably, a flex-PCB will involve a stiffener in areas where mechanical strength is required, e.g., its connection with other electronics (e.g., the transmitter board). The stiffener can be for example 75 μm (3 mils of FR4) or a similar thickness of stainless steel or Polyimide.

The bonding pads of the flexible connector 3 can be made with different finishes including Electroless Nickel, Immersion Gold (ENIG), Electroless Nickel, Electroless Palladium, Immersion Gold (ENEPIG), Immersion Gold on Cu directly as few examples. The pads are used on one end to connect with the integrated sensor circuit 1 and on the other end with the transmitter 2 using different methods detailed next.

A variety of processes can be used to join the connector 3 to the monolithic sensor circuit 1. In accordance with some embodiments of the disclosure, a two-wire connector 3 is made up of a flexible polymer substrate, e.g., polyimide or parylene-C, with thin copper traces (e.g., 20-100 μm wide, alternatively 20-50 μm wide; and 20-100 μm apart, alternatively preferably 20-50 μm apart; with 1-30 μm thickness, alternatively 10-30 μm thickness). The copper traces are sandwiched by the polymer substrate to avoid exposure to bodily fluids. On the implantable multianalyte monolithic integrated circuit 1 side two bonding pads are created by removing a passivation layer during a CMOS manufacturing process, removing any biocompatible membrane and/or polymer and plating gold on top of the copper wire connection on the circuit. At the connection site to the implantable multianalyte monolithic integrated circuit 1, the copper traces can be exposed and covered with gold for good connection to the sensor using a flip-chip bonding technique or wire bonding.

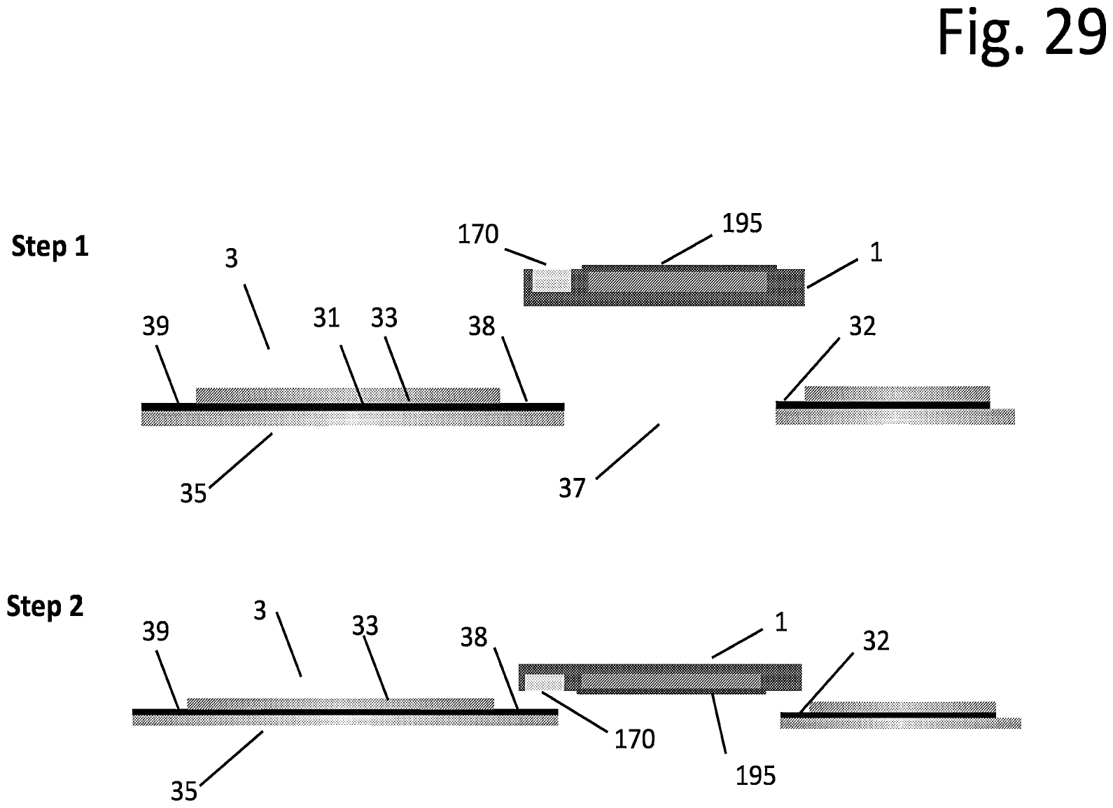

FIG. 29 (step 1) provides a packaging process scheme of the sensor circuit 1 onto the connector 3 (or wire 3) which may be flexible. FIG. 29 (step 1) illustrates a sensor circuit 1 with contact pads 170 and a protective layer 195 positioned adjacent to a connector 3 (e.g., a flexible PCB panel). The connector 3 may include conductive traces 31 in a conductive layer of the flexible PCB, i.e., under a PCB top insulation layer 33. The part of the conductive trace that is adjacent to the distal end and is not covered by the insulator 33 is called the contact pad on the sensor side 38 and the part away from that that is not covered with the insulator layer is called contact pads 39 for the interfacial connector. The sensor circuit 1 can include multiple sensors 160 (or sensing elements) at some distance from CMOS pads 170. It also shows the flexible connector wire 3 further comprising a flexible substrate 35. Under the other side of the CMOS device and beyond a cut out 37 in the flexible PCB may lie another chip side contact pad 32 on the flexible PCB.

FIG. 29 step 2 shows how the CMOS device 1 is aligned with the flexible connecting wire 3 such that the contact pads 38 on the Flex are aligned with contact pads 170 on the chip. Moreover, the extra pads 32 on flex is aligned with extra pads or other structures on the chip. These extra pads on both sides are optional and can be used for electrical connection or only for mechanical strength. This alignment can be done with systems known as wafer aligners or other alignment systems. The remaining system components i.e., the flexible substrate 35, the flexible top insulation 33, the connector contact pads 39, the protection layer 195 remain.

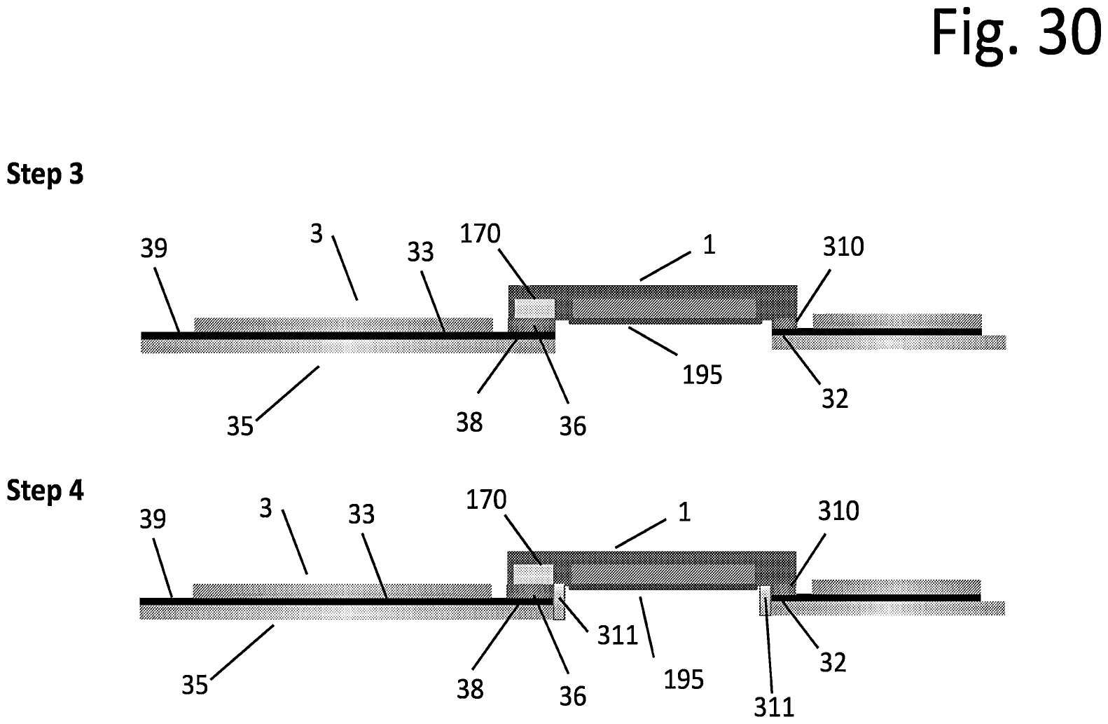

FIG. 30 step 3 shows a conductive material 36 can be used to electrically connect the contact pads of CMOS device 1 with the corresponding contact pads of flexible connector 3. Another bonding material 310 (can be same as 36 or different) is used to attach the extra contact pads 32 on the flex side with the corresponding pads or structures on the CMOS side. The contact pads on either or both sides can have small “bumps” or a multi-layer conductive film (e.g., ENIG or ENEPIG) to improve the adherence. In addition, conductive materials like Anisotropic conductive epoxy or anisotropic conductive adhesive (ACA) can be used to support the adhesion and conduction between the two devices. The bumps on both sides can be joined together either by cold or hot processes by a combination of mechanical (e.g., force, ultrasonic), thermal (e.g., micro-welding, hot fusion), and related methods. The remaining system components i.e., the flexible substrate 35, the flexible top insulation 33, the connector contact pads 39, the protection layer 195 remain.

FIG. 30 step 4 shows how the bonded contact pads are sealed with insulating material 311 to ensure no test liquid can reach the contacts pads area. This can be done using different methods including pray coating with a stencil (to protect the areas that should not get the insulating layer), pico-droplet dispensing, lithography etc. The remaining system components i.e., the flexible substrate 35, the flexible top insulation 33, the connector contact pads 39, the protection layer 195, and the additional bonding material 310 remain.

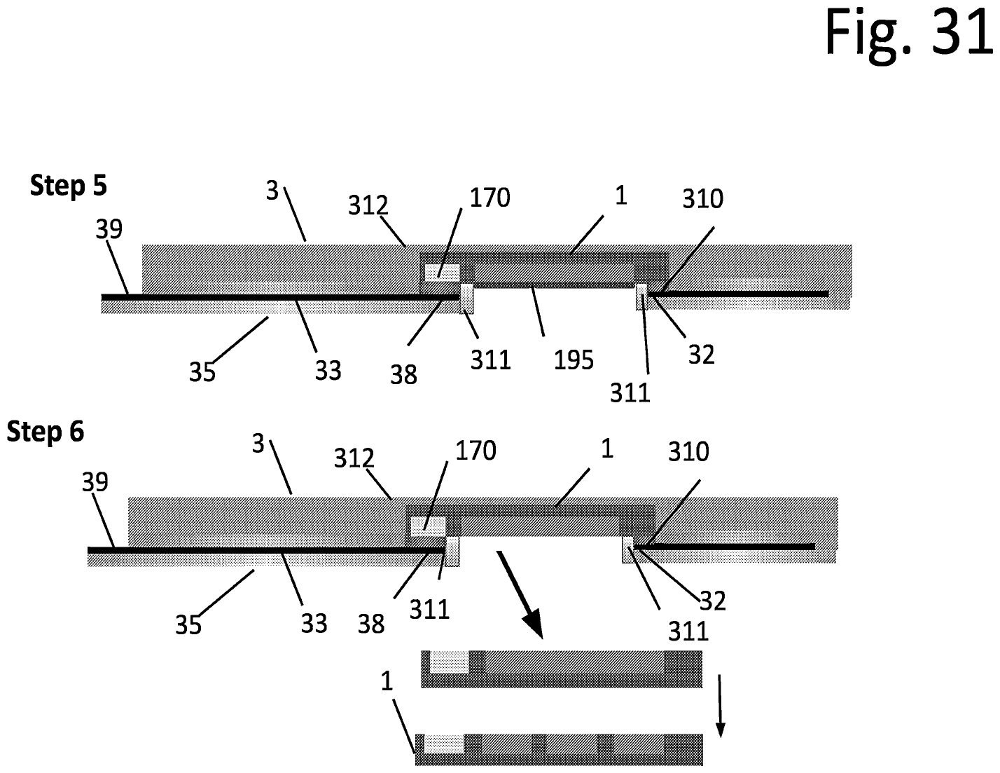

FIG. 31 step 5 shows how a backside overcoat, or a similar coating process can be used to provide a protective backside coating 312 of the packaged device. This can be done by several methods like spray coating polyimide and baking (e.g., thermal, UV, combination). The remaining system components i.e., the flexible substrate 35, the flexible top insulation 33, the connector contact pads 39, the protection layer 195, the additional bonding material 310, and the sealing material 311 remain.

FIG. 31 step 6 shows the resulting device with a CMOS chip packaged within a flexible PCB. It also shows the removal of the CMOS surface protection layer 195 at this step using chemical or dry etching (e.g., Oxygen Plasma). The remaining system components i.e., the flexible substrate 35, the flexible top insulation 33, the connector contact pads 39, the additional bonding material 310, the sealing material 311, and the backside coating 312 remain.

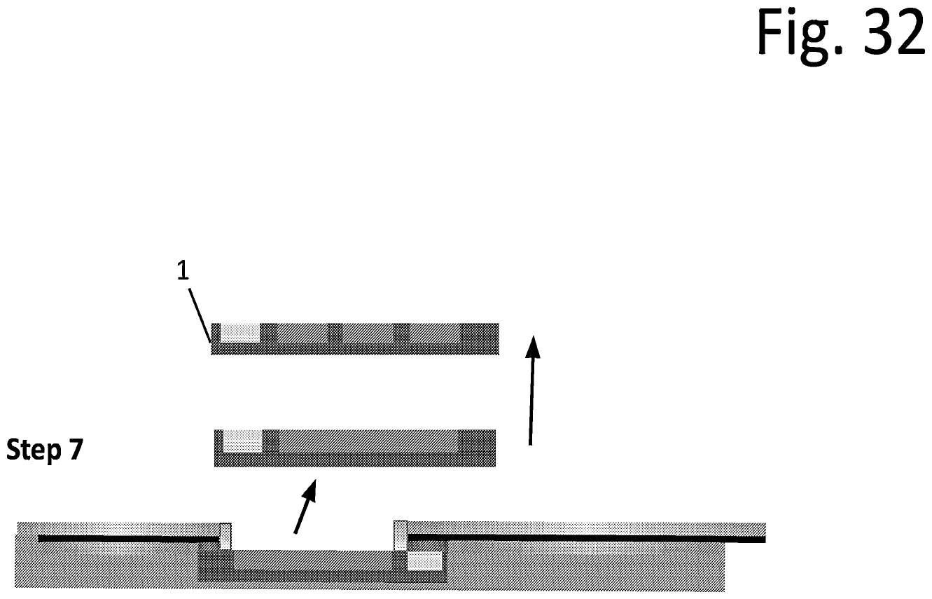

FIG. 32 shows how the device in the last step (FIG. 31 step 6) can be turned upside down to represent a packaged device.

A) Wet Chemistry and/or Polymer Deposition on Flex

It is possible to coat the CMOS devices with chemistry (one or more layers) either before or after packaging those with the flexible PCB. For example, in one embodiment, the CMOS wafer can be coated with different chemistry layers after postprocessing and coating the surface with suitable conductive materials. This wafer-scale chemistry coating has several advantages in terms of scalability and repeatability. In this case, the chemistry needs to be protected from the rest of the processing (e.g., packaging with the flex PCB). Also, the rest of the packaging needs to avoid processes and process conditions not suitable for the chemistry layers. For example, the packaging of CMOS chips with the flexible PCB process needs to avoid high temperatures that can negatively impact the surface chemistry. For example, the attachment of the flex and CMOS device needs to use cold bonding (e.g., only using mechanical energy). The insulation layers formed afterwards also needs to avoid high temperature processes and either use UV curing or localized heating (e.g., using lasers or photonics).

In some embodiments, the surface chemistry layers can be coated after the CMOS wafer is cut and the sensor chips are packaged with the flexible PCB. In this case, the chemistry layer is less exposed to further process steps. Tis methods can be scaled using roll-to-roll manufacturing scheme. The packaged devices can be coated with the chemistry either in the form of a panel of devices or as individual devices.

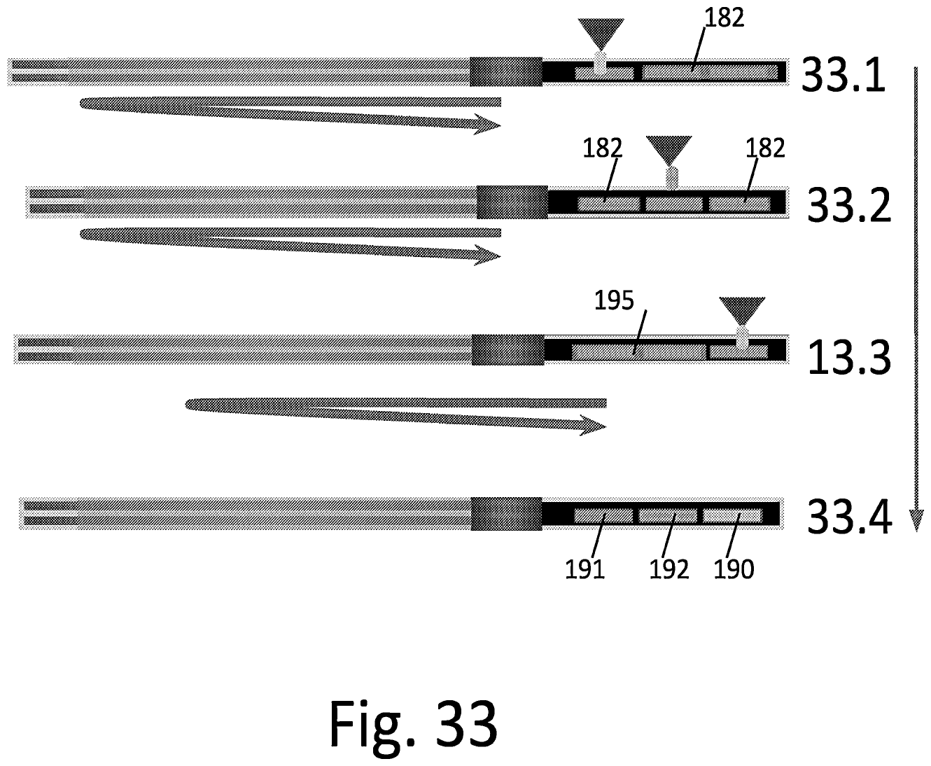

FIG. 33 shows the process steps to coat a packaged sensor (CMOS device bonded with the Flex PCB). It shows a simplified view of the sacrificial layer-based process to coat three integrated sensing element electrodes with three different chemistry layers. Depending upon the chemistry layers, the process can be done without the sacrificial layer in which the patterning layer itself can be used to protect the parts of the device that should not be coated with the chemistry layer being used in that particular process step. The chemistry layers can be formed using spin coating, spray coating, or dip coating. At the end, the packaged device with functional coatings is shown in FIG. 33 step 4 (33.4).

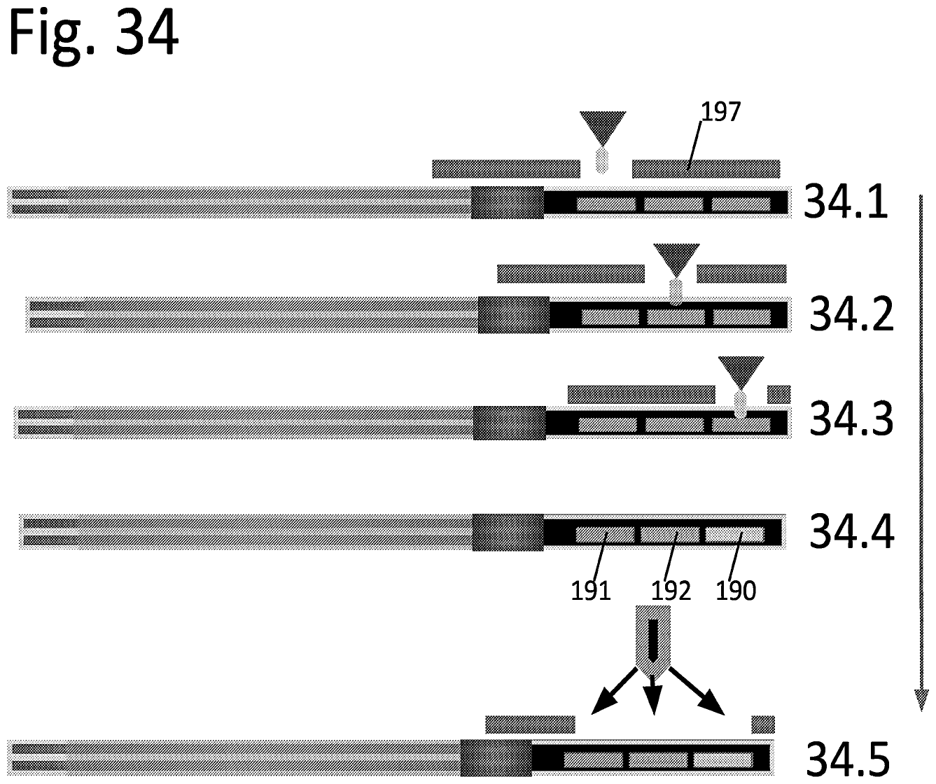

FIG. 34 shows the process steps to coat a packaged device with three different chemistry layers using a pico-droplet dispensing together with a stencil 197 to avoid cross-contamination. The process can be used to coat the device with as many chemistry layers as desired. FIG. 34 step 4 shows the device coated with three functional layers (191,192,190). As an additional step, the device can be coated with a membrane and/or biocompatible layers as discussed before. As an example, a spray coating method to cover the sensing electrodes and protecting the rest of the packaged device (using stencil or screen) is shown in FIG. 34 step 5 (34.5).

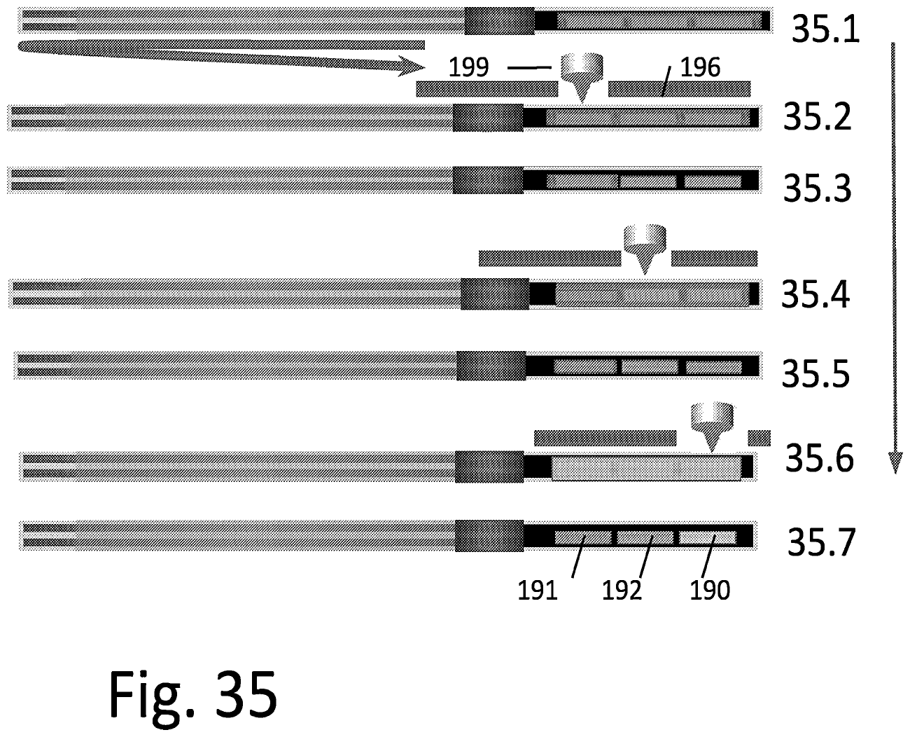

FIG. 35 shows a process in which a photo-definable hydrogel is used to cover selective parts of the CMOS device (shown after packaging but can also be used before packaging). In this case, a photo-sensitive (e.g., Photo-polymerizable) material (e.g., poly-HEMA, PEG-DA with a Photoinitiator) is used to cover the CMOS device. Then suitable light (e.g., UV) is shone on the device through a suitable photomask 196 through a suitable light source 199 which allows light to go through in certain areas and block it from the rest of the areas. The parts where light goes through to the hydrogel, it crosslinks the hydrogel and hence those parts remain. The rest of the parts are dissolved in water when the whole device is placed in water. This process can be repeated to cover the device with one or more layers of surface chemistry.

### Transmitter

The analyte concentration measurement system requires an external transmitter 2 located on top of the skin of the user to secure the transdermal connector 3 and the monolithic sensing circuit 1. The transmitter 2 can provide electrical power to the multianalyte monolithic integrated circuit 1 via the flexible connector 3. The external transmitter 2 can be used to power monolithic integrated circuit 1 and to communicate with implantable multianalyte monolithic integrated circuit 1, i.e., to receive data from and send data (e.g., commands) to sensor before and after the multianalyte monolithic integrated circuit 1 is inserted.

A transmitter of an embodiment of the instant disclosure is unique such transmitter that processes digital signals from a digital glucose sensor, as compared to the ones processing analog signals from passive sensors.

The transmitter is an electronic device with a power source (e.g., a battery). A printed circuit board (PCB) can be used to make a transmitter system with all components to function as the transmitter 2. One or both of thick (rigid) and thin (flexible) PCB technologies can be used, depending upon the application.

A schematic diagram of an example of a transmitter according to some embodiments of the disclosure can be seen in FIG. 5. The transmitter 2 can include a transceiver 210 that can generate and detect communication signals flowing over a combined power/data connection through wire 3, a high-performance digital microprocessor unit 211 for system control, a low-energy wireless communication chipset 213 (e.g., Bluetooth), an antenna 218 for wireless communication (e.g., Bluetooth antenna), a power management unit 214 connected to a battery 212, and a voltage regulator unit 217 to provide regulated power to the implantable multianalyte monolithic integrated sensor circuit 1.

In some embodiments, the transmitter can be made using a system-on-chip (SoC) 260 that combines several functions including the wireless communication transceiver, the microprocessor, the analog frontend, power management circuit, etc.

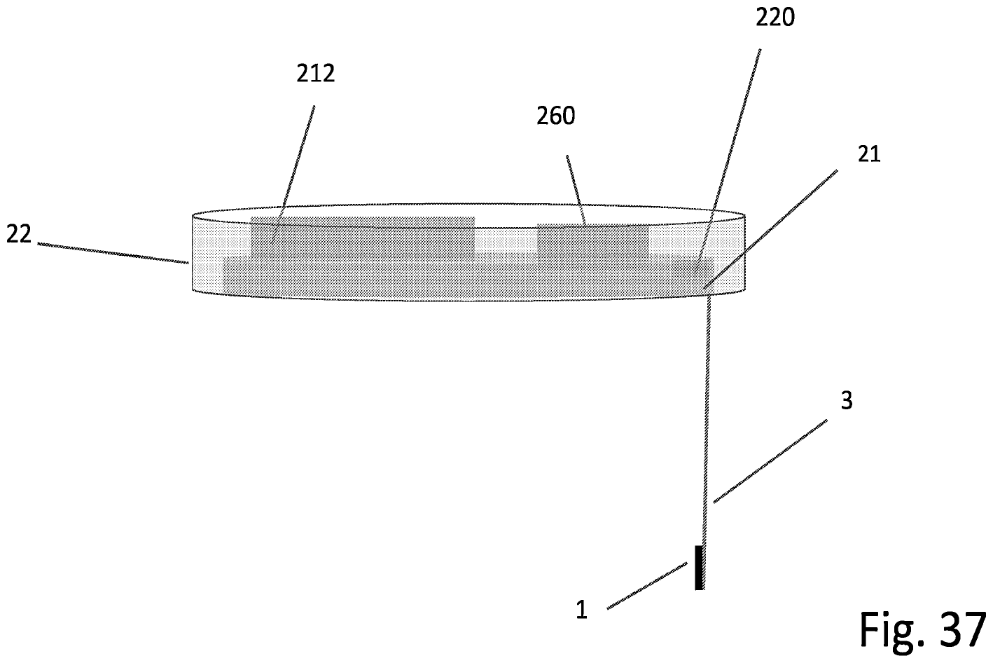

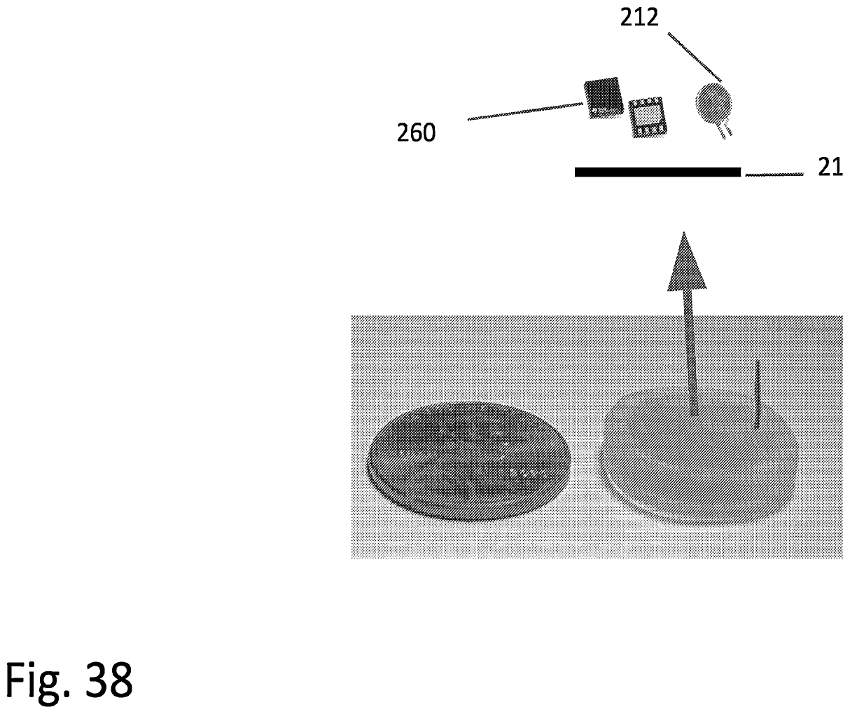

An example of such a transmitter is shown in FIG. 37. It still uses a battery 212 to power the system. The transmitter 2 can be formed on a printed circuit board 21 that has all the above-mentioned components and lies within a sealed housing 22. The sensor 1 is connected to the transmitter 2 via the flexible connector 3 which connects into an interface connector 2. The SoC can use ultra-low-power design which enables use of extremely small and even flexible batteries.

In an exemplary commercial off-the-shelf transmitter design, a system-on-chip can be used to provide the functions of a microprocessor, a wireless communication link (e.g., BLE) with an integrated antenna, as well as on-system memory. Similarly, a battery unit can contain the battery along with a battery management circuit. A standard coin cell battery is possible. Examples of a suitable system-on-chip for use in the transmitter include TAIYO YUDEN® EYSHSNZWZ BLUETOOTH® Low Energy Module and Lilypad coin cell battery module. Possible SoCs include also, for example, nRF52, DA14531, HJ-131, etc.).

Additional electronic circuitry like a transceiver can be implemented to pre-process the incoming data from the sensor circuit 1 to make it easier to be read by the processor. In one example, the transceiver may include operational amplifiers (e.g., Texas Instrument TLV9061) to amplify the incoming signal and a comparator (e.g., Texas Instrument TLV7011) to create a rail-to-rail signal. In some examples, this transceiver circuit can be implemented within the SoC e.g., by using on-chip amplifiers and comparators.

The transmitter can also include usability features like a temperature sensor to detect environment temperature reading which can then be compared with the temperature sensor on the integrated sensor 1 to detect change in temperature from the environment to the tissue around the sensor. Another optional embodiment is for the transmitter to have a light sensor (e.g., a Photodiode) that acts as a way for the system to detect its state (i.e., still in the packaging, vs outside of the packaging).

Although a transmitter could be implemented via a commercial off the shelf technology, it is possible to use a custom solution using application specific integrated circuits (ASIC) or a custom System-on-Chip (SoC).

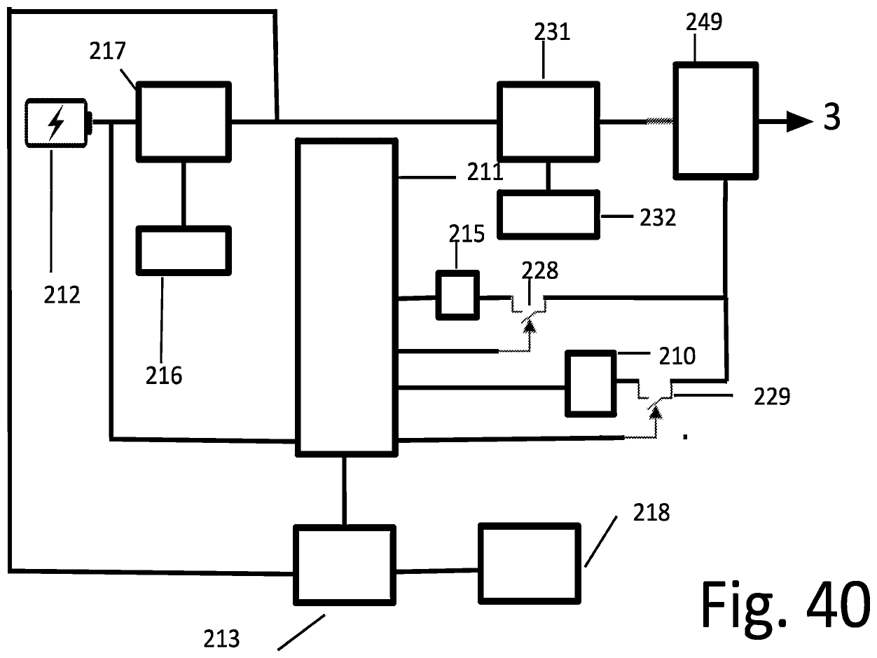

FIG. 40 shows a detailed electrical schematic of a transmitter embodiment which can be fully integrated into a custom ASIC or SoC. The ASIC may include a battery 212, a voltage regulator 217, a voltage reference 216 (can be within the, a microprocessor/microcontroller 211, a wireless communication transceiver 213, an antenna 218, wire 3, a DC decoupling capacitor 242, a switch 228, a switch 229, an Rx unit 210, a Tx unit 215, a secondary voltage regulator 231, a voltage reference circuit 232, and resistor 243.

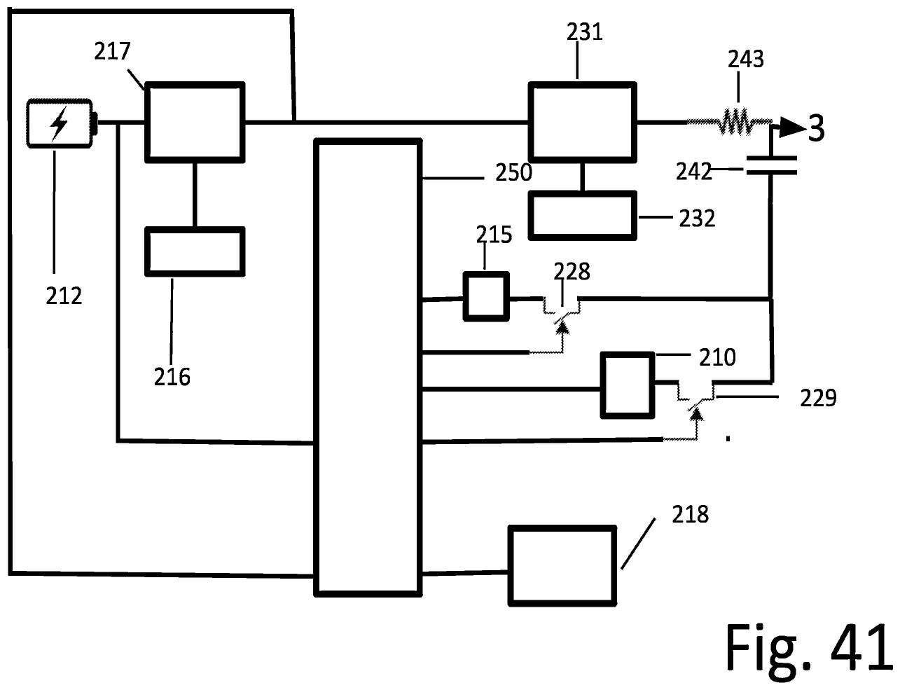

FIG. 41 shows another ASIC or SoC embodiment in which a battery 212 powers the system via a voltage regulator 217 that uses a voltage reference 216 to set the output voltage. Different types of batteries can be used to power the system, including Lithium-ion, Silver, Zinc, Lithium polymer, thin-film etc. For example, a lithium-ion battery voltage can range from 3.3V to 2.8V based upon remaining charge. The voltage regulator can provide a stable output voltage (e.g., 3V) independent of such battery status, thus helping with stable circuit operation for longer duration. The voltage regulatory powers the rest of the transmitter components including an SoC 250 including a microprocessor/microcontroller and a wireless communication transceiver (e.g., BLE transceiver) feeding an antenna 218. The microcontroller generates a suitable tag signal to communicate with the receiver (implantable monolithic sensing circuit 1). The tag signal is fed to a TX unit 215 that shapes the tag and couples it to the wired connection 3 via a DC decoupling capacitor 242. A switch 228 is closed when this tag signal is being transmitter while another switch 229 is kept open to prevent leakage of this signal into the Rx unit 210 of the transmitter. After the tag is sent and a certain duration is passed, a signal is expected from sensor 2. At this time, switch 228 is opened while switch 229 feeding the signal to the Rx unit 210 of transmitter 2 is closed. The Rx unit 210 can shape the received signal, e.g., by using a monostable circuit to increase the width of the received signal. The received signal passes through the DC decoupling capacitor 242 before being fed to the Rx unit 210. The main voltage regulator 217 feeds power to a secondary voltage regulator 231 that is used to generate a more suitable voltage (e.g., 2V) for the receiver (e.g., implantable monolithic sensing circuit 1) using voltage reference circuit 232. The resistor 243 is used to isolate the voltage regulator from the transmission line 3. The resistor 243 and the capacitor 242 are example embodiments and can be replaced with other types of electrical networks to perform this same function of combining the data with the power on one end (from transmitter to the sensor) and separating the data and power on the other end (from sensor to the transmitter).

In some cases, the SoC and the battery can be further integrated. This can reduce the size of the device even more. An example embodiment with this design is shown in FIG. 40. In another embodiment, the integration can even be done at the chip scale. Such batteries have lower energy density but can be useful for extremely efficient and low-power designs. In some cases, such low power batteries can be used to power the sensor continuously since it uses extremely low power. Wireless communication typically requires more power which can be derived from a different source e.g., from an NFC or RFID link with a reader device.

In some embodiments, the transmitter can be formed using a PCB while avoiding the need for an interfacial connector (e.g., a Zero-insertion force or ZIF connector) for the flexible connecting wire 3 that has the integrated sensor 1 on its distal end. This is achieved by using a rigid-flex PCB design in which a rigid PCB and a flexible PCB are attached without the need for a bulky connector. A rigid-flex PCB can be designed in which the transmitter 2 uses the rigid part while the flex part is used for flexible connector 3.

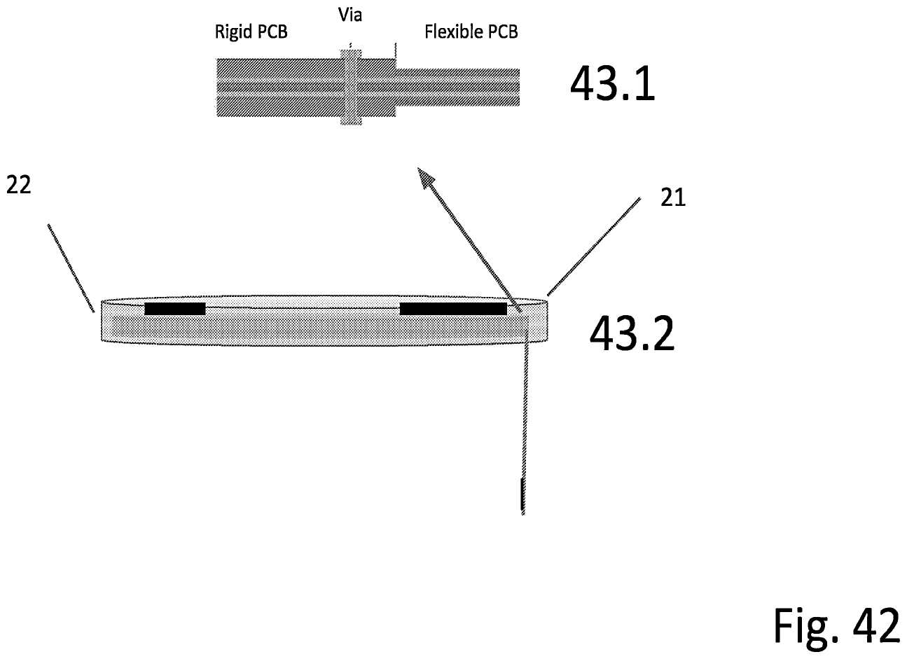

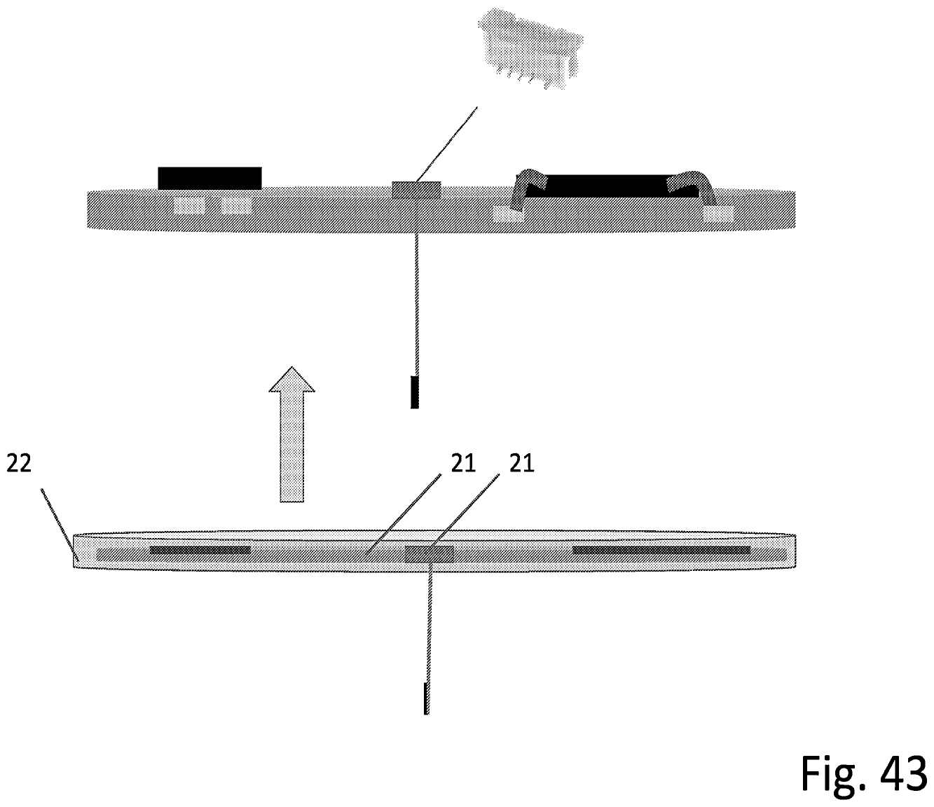

One embodiment of a rigid-flex PCB design is shown in FIG. 42. Example materials for rigid board are FR4 and Rogers material whereas the example materials for flexible board are Polyimide and Liquid crystal Polymer (LCP). An example embodiment will use a 2 conductive layers process on both the rigid and the flexible PCBs. The flex PCB is connected to the rigid PCB via an interlayer-via which connected both sides electrically. This avoids the need for an interfacial connector which simplifies device design. The flexible PCB can be sandwiched between two rigid PCBs or can be attached to a single rigid PCB. The flex PCB will have between 1 to 6 conductive layers whereas the rigid PCB will have between 1 to 6 conductive layers. Both use different types of insulating layers (e.g., Polyimide for Flex, Solder mask for FR4) and the number of layers depends upon the number of conductive layers (e.g., 3 insulating layers for 1 conductive layer PCB). The flex will have to be able to bend up to 90 degrees (as shown in FIG. 43.2) to be able to fit in a needle for insertion. In some embodiments, the transmitter can be formed using a flexible PCB substrate with electronic components which avoids the need for a rigid PCB. This makes the transmitter lighter and smaller. The use of flexible substrate instead of rigid substrates would enable a thinner and more flexible transmitter that conforms better with the skin. The flexible transmitter uses electronics components similar to the rigid substrate. It also has an interfacial connector available for attaching the flexible connector wire 3 that has the integrated CMOS sensor 1 near its distal end.

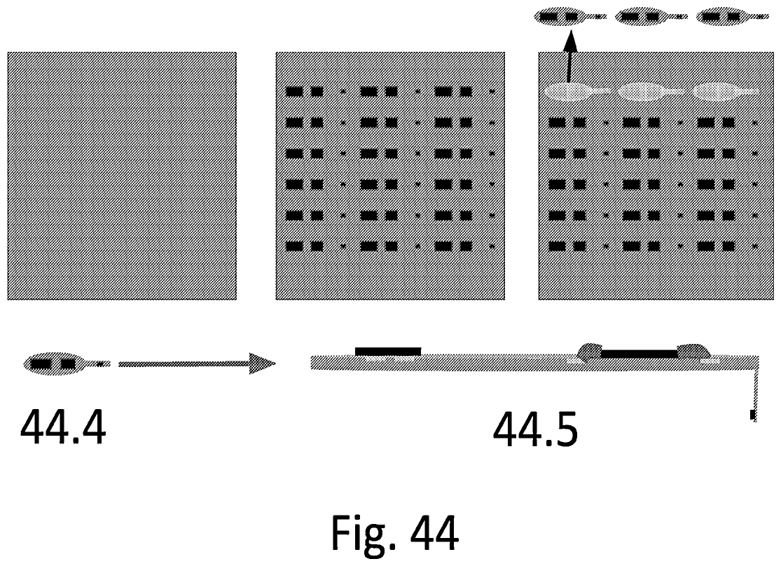

An example of a flex-based design with an interfacial connector is shown in FIG. 44. The flex PCB specs used for the transmitter and those used for the flexible connector 3 can be different. For example, a thicker substrate and thicker Cu can be used for the transmitter whereas thinner layers can be sued for the flexible connector 3 that interfaces with the CMOS sensor 1.

In some embodiments, a single flexible PCB substrate can be used for both the transmitter PCB and the flexible connecting wire 3 such that the fabrication process is extremely simplified. In such an embodiment, the intermediate interface connector can be eliminated. Both the transmitter and the sensor can use chip-on-flex, flip-chip, chip-in-flex packaging to attach electronic components to the PCB substrates. The resulting device can be cut into precise shape using suitable cutting schemes (e.g., laser dicing). In one embodiment, the flexible connector wire 3 segment can be made using higher flexibility while the transmitter part would be made with lesser flexibility (to retain its shape and mechanical integrity under stress).

An example embodiment of a single flexible PCB substrate transmitter can be seen in FIG. 44. The flexible connector 3 part will be able to bend up to 90 degrees as shown. To improve flexibility, the part that needs to bend is kept thin and uses minimal layer thicknesses. The flexible PCB may have 1 to 6 conductive traces. The minimum trace width can be 1 mil whereas the maximum trace size can be 10 mils. The Cu used can typically be from 0.25 Oz to 2 Oz. A thinner or thicker Cu can be used in parts of the device. Cu thickness can be controlled by controlling plating time and locations. The rigidity of flex can be controlled by controlling the number of layers and the use of layers in certain sections of the board (e.g., not using bottom Cu in the areas where flex needs to bend).

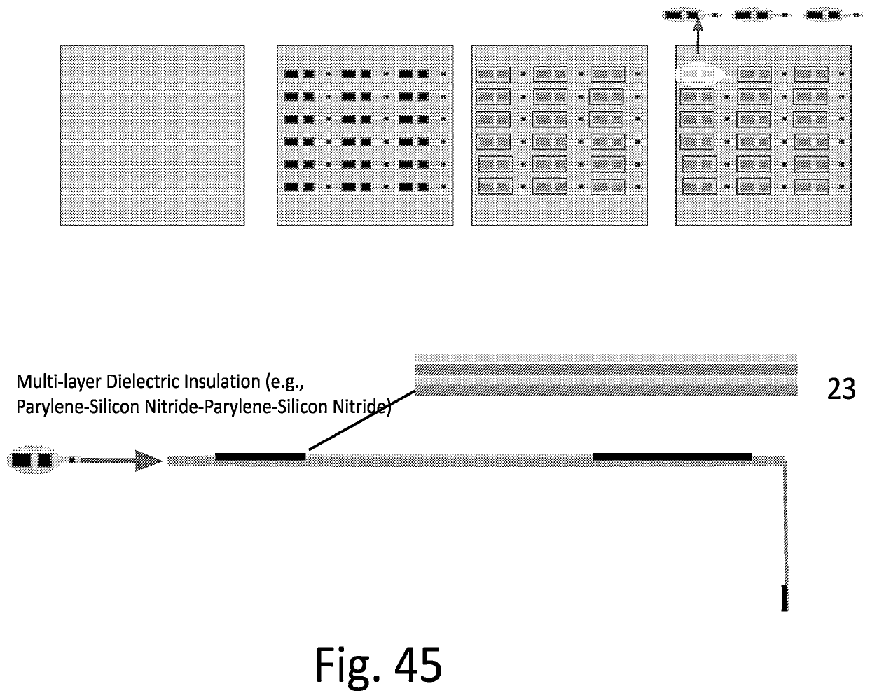

The transmitters shown so far need to be encased in some insulating casing for environmental protection. In some embodiments, this can be avoided. In some embodiments, the flexible PCB substrate can use materials not used typically in standard electronics industry to optimize device design. For example, a multi-layer insulating coating can be used to cover the electronics on a flexible transmitter and sensor such that only the sensing elements of the integrated sensing chip are exposed to the sensing solutions. An example of such an embodiment is shown in FIG. 45. An example of such a coating is a multi-layer dielectric coating using a stack of Silicon Oxide and Parylene to insulate the electronics from environmental variables. Alternatively, a thicker insulting coating (e.g., Rubber Silicone) can be used. The multilayer insulator can be from 5 um to 500 um whereas the thicker silicone casing can be from 100 μm to 1000 μm. This eliminates the need for a plastic casing. This enables an extremely thin and flexible transmitter that is gentle on the skin.

The transmitter can wirelessly communicate the data to a hub or smart device (e.g., a phone, a tablet, or a special separate device 4). In accordance with some embodiments, the hub or smart device can be connected (either by wire or wirelessly) to a cloud server via a network (e.g., the Internet, a private network such as virtual private network (VPN), or a public network). The transmitter uses a low-power wireless communication technology (e.g., Bluetooth, Zigbee) to communicate with the smart device. The transmitter may use a standard BLE profile (e.g., the CGM profile) to enable a simple interface with other devices (e.g., to form artificial pancreas). A secure BLE connection can be established between the transmitter and the host device using techniques like dedicated digital keys.

In various embodiments, the transmitter 2 can communicate with the monolithic integrated circuit 1 via the flexible connector 3. The communication can be bi-directional or unidirectional, wherein optionally bi-directional communication is sequential, meaning that first the transmitter sends a command (e.g., a tag) to the multianalyte monolithic integrated circuit 1 to trigger analyte measurement by the sensor. After the transmitter sends this tag, it can go into receiving mode (i.e., it waits for the sensor to send it the measured data). Error correction schemes can be employed to minimize the error in this communication. Parity-bit based designs and more advanced error correction codes can be used as well. Different types of modulation schemes can be employed for this communication.

The transmitter is programmed using a firmware to enable operations desired in a typical implementation. The firmware is designed to perform the operations necessary to control the operation of the transmitter hardware to match with system needs. The firmware is stored on a permanent memory block on the transmitter. The memory typically resides within the Bluetooth module of the transmitter. Many BLE modules have the option to wake-up the system using an NFC link. For example, the BLE module from Taiyo Yuden has embedded NFC capability to use wake-up in the field. The transmitter firmware is designed to keep it in low power mode until any such wake-up calls are received. This can enable the transmitter to remain in low power mode till the user is ready to operate the unit (e.g., after sensor insertion). Then the transmitter can be taken out of sleep mode to start a secure dedicated connection with a desired reader and start communicating the data.

After such wake-up, the transmitter firmware (designed as a BLE client) looks for a BLE master to connect to. If such a connection (device) is available, the transmitter establishes a connection to the device (e.g., smart reader). After this, the transmitter tests the available power level and upon validation of a stable power supply, it looks to see if a suitable sensor is attached (via the amount of current drawn). It also checks sensor readings to be within normal range with a normal rate of change (as programmed in its memory during manufacturing) to confirm if the sensor is operating properly (e.g., in warm-up period). After the warm-up period is complete (prep-programmed during manufacturing), the transmitter checks the reading values and the rates of change to determine if the sensor is operating properly.

Since the sensor is a smart CMOS device, the data coming from it is different than the data coming from the passive CGMs in other applications (just the sensor current). In this case, the header (preamble), the footer contains the sensor batch and type and are compared against the IDs stored on the transmitter to confirm that the sensor and the transmitter are indeed matching. Moreover, the error detection schemes (e.g., CRC) can quickly tell if the received data is properly encoded. The data from the sensors, including the temperature sensor, can then be compared against each other and the factory stored values (e.g., in a look-up table) to confirm if the sensor is completely operational and is properly inserted in the tissue. After this, the transmitter sends a tag to the sensor telling it to start sending the sensor data at a desired interval (e.g., once every 2 seconds). This data is then read by the transmitter, tested, and combined (e.g., averaged over 3 sensors) before sending it to the reader at a certain frequency (e.g., once/minute).

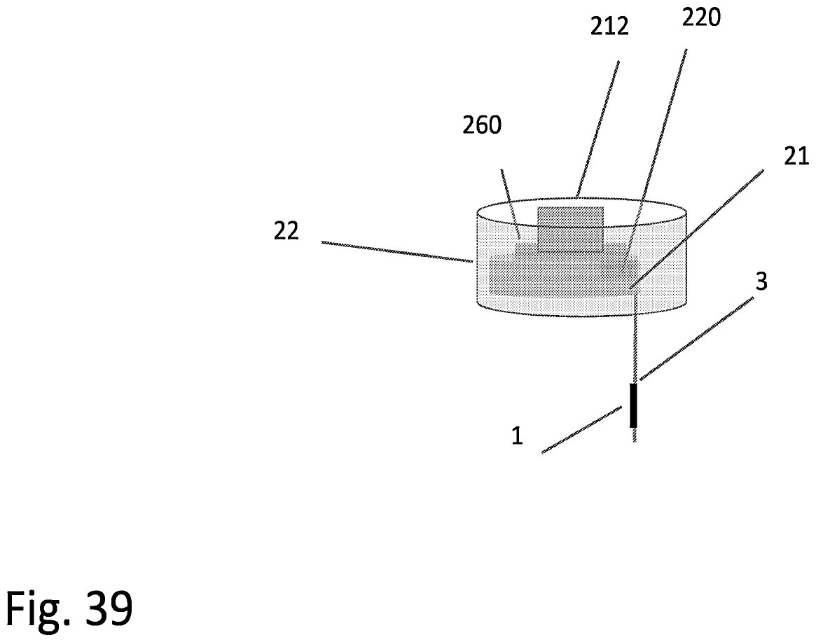

The transmitter 2 includes some programmable circuits (e.g., a microcontroller) that are programmed to control the operation of the hardware. A scheme for such a program known as the firmware is shown in FIG. 39.

### System Packaging and Operation

The sensing platform can be loaded inside an applicator device and the entire assembly can be sterilized (e.g., by Synergy Health (San Diego, CA)). To sterilize the device before embedding it inside the body, conventional methods of sterilization (e.g., steam, Ethylene Oxide) can be utilized. In one particular embodiment, Electron-beam (e-beam) sterilization can be used to sterilize the sensor as well as the applicator once the sensor is pre-loaded in the applicator. The underlying electronics can be designed to be resilient to e-beam radiation. The enzyme chemistry can be characterized to calibrate for any changes in the enzyme chemistry response due to sterilization. In one embodiment, 25 kGray of e-beam irradiation can be sufficient to sterilize the sensor without impeding its function. Sensors can be placed inside the applicator and then the whole assembly can be sterilized. In a different embodiment, the integrated sensor 1 and the connector 3 assembly, the transmitter 2, and the applicator can be sterilized separately using different methods (e.g., sensor 1 and connector 3 assembly using e-beam, transmitter 2 and applicator using ethylene oxide) followed by sterile assembly.

One or more sensors 1 can be placed in desired tissue locations using an applicator as noted above. As noted throughout the present disclosure, in embodiments, the implantable multi analyte monolithic integrated sensor circuit 1 can be used to measure glucose levels in the user. The readout procedure for collecting glucose data from the implantable multianalyte monolithic integrated circuit 1 can start with energizing the implantable multianalyte monolithic integrated circuit 1 through transmission of power signal (through wired connection) from the external transmitter 2. The external transmitter 2 can be configured to select the appropriate powering mode (continuous/intermittent) based on user or clinician input.

The external transmitter 2 can receive sensor data, display sensor data, store the data and relay it to a smart device 4, or send it to a remote server. External transmitter 2, smart device/communication device, or remote server can relay and process the sensor data in a manner commensurate with its processing, storage, or battery capability. The data processed in external transmitter 2, smart device/communication device, or remote server can be relayed to external transmitter 2, smart device/communication device, or remote server to provide, display, or store, information (e.g., blood glucose levels, daily trends) or predictions thereof or suggestions (e.g., behavioral changes, treatment changes) based on sensor data or predictions.

After the sensor assembly is removed from the packaging, the transmitter 2 needs to be turned on to power the sensor 1 and to communicate with the reader 4. Different schemes can be used for this purpose. For example, a photosensitive detector (e.g., a photodiode) can be used to detect the opening of the package. This signal can then be used to turn on the transmitter power supply and start the initialization and connection procedure.

In a different embodiment, a wake-up-in-the-field method using an NFC pairing capability embedded in the transmitter can be used to turn on the transmitter operation after the user opens the sensor packaging. The transmitter is paired with an NFC enabled device to send it the turn-on signal. The transmitter then starts the initialization process.

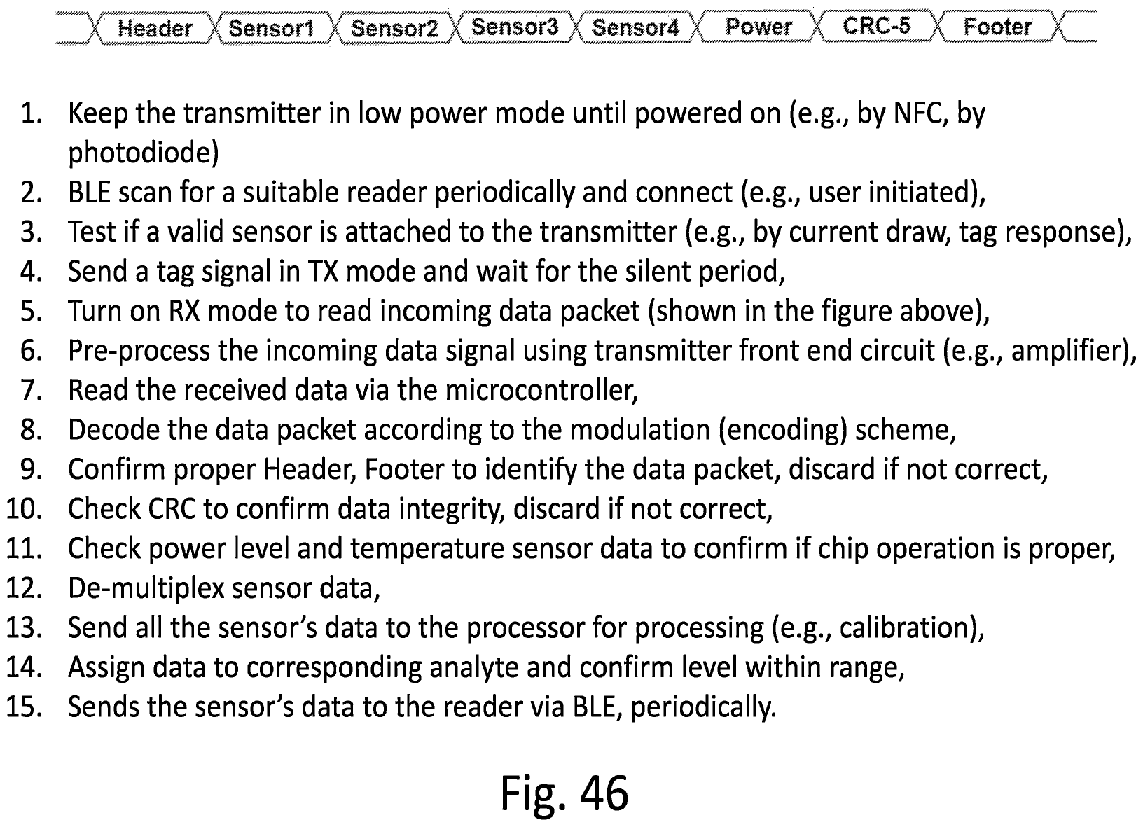

FIG. 46 shows an algorithmic scheme to program the processor (e.g., a microcontroller) in the transmitter (which acts as the brain of the transmitter) with a firmware to control its operation. It shows that the transmitter (microcontroller) is programmed to stay in a low power (e.g., deep sleep) state. After the user opens the packaging and performs a turn-on operation (e.g., either by using an NFC device to pair with it) or the transmitter automatically detects the user's intent to use it (e.g., by detecting a change in background conditions like light via a photodiode), it performs an initialization sequence which includes a self-test as well as a scan for the reader via BLE. Once it finds a matching reader, it connects with it via BLE. Next, it tests if a good sensor is connected by performing electrical measurements (e.g., voltage drop, current draw) and by sending a command signal and testing the response. After that, it starts transmitting the tag signal, waits, and starts reading the corresponding data from the sensor chip in RX mode. The analog frontend of the transmitter pre-processes the incoming data signal separating the power and data signals (via mux/demux) and sends it to the microcontroller, which processes the data (e.g., decodes according to the modulation scheme, confirms proper header, footer), checks if it detects any error (via error detection/correction code), checks power level, and de-multiplexes the data, e.g., separates the data from all sensors (e.g., 3 electrochemical and 1 temperature) sensors. It then sends the data to a microprocessor for further processing. It also tells the reader which data corresponds to which sensor and hence to which analyte.

The system can be easily expanded by having multiple sensors on a single device. For example, a device can have 8 sensors in total: 3 sensors each for glucose and lactate, one for background reference, and one for system integrity (negative control). Another system may have 9 sensors in total, 3 each for glucose, lactate, and ketones. An example of such a design is shown in FIG. 36. The integrated sensing electrodes provide sufficiently large current, and the integrated electronics have a high resolution. Hence, a large number of sensors can be added to the design. In another embodiment, the system can be extended to have 3 sensors each for glucose, lactate, and ketones in addition to having a background reference sensor and a negative control sensor.

Application of multivariate calibration to single analyte sensor, or a multi-analyte sensor, with sensing elements that have non-identical sensitivity will be explained here. Such multivariate calibration can be performed using a matrix method (as explained in UNCERTAINTY ESTIMATION AND FIGURES OF MERIT FOR MULTIVARIATE CALIBRATION (IUPAC Technical Report) Pure Appl. Chem., Vol. 78, No. 3, pp. 633-661, 2006. Incorporated herein by reference in its entirety), a machine learning method, or any multi-parameter model that could be developed by those skilled in the art.

In an embodiment where each of the sensing elements are coated with chemistry for one analyte and have different sensitivities (e.g., due to different coating thickness), a relative sensitivity matrix can be generated and used to discern the change in signal due to analyte and the change in signal due to interference or noise (e.g. drift, instrument noise, foreign body response, sensor encapsulation)

Similarly, in an embodiment where a multitude of sensing elements are functionalized with a multitude of sensing chemistries, provided that there is a difference in relative sensitivities to different analytes, the relative sensitivities can be used to calibrate the sensor for different analytes and interferents.

This can be generalized to larger arrays and allow sensing elements to shrink beyond technical, manufacturing, financial ability to selectively functionalize each sensing element and achieve sensitivity to more analytes than the patterning technology allows.

An embodiment could forgo patterning completely granted that each of the sensing elements are functionalized with different sensitivity to analytes due to the variations induced or inherent in the functionalization process. This would save manufacturing complexity yet require calibration of each sensor as opposed to a representative subset of sensors.

### EXAMPLES

### Example 1—Sacrificial Layer Patterning

Materials used include Polyacrylic acid from Polysciences—03326-250 Poly (acrylic acid), 25% soln. in water [MW˜345,000]—(“PAA”), Glucose oxidase enzyme (“Gox”) Glutaraldehyde, AZ 300 MIF Developer, Photoresist AZ5214E Lithography Mask, UV light source, Argon gas, DI Water. The process starts by diluting PAA solution from 25% to 5% using DI water. Next, we deposit 20 ul of solution onto the sample (e.g., CMOS device) and Spin @ 500 rpm for 10 seconds until a thin layer forms. The sample is stored in incubator (35 Degree Celsius) for 1 hour for the PAA layer to crosslink. Next, we deposit and spin Photoresist AZ5214E @ 2500 rpm for 20 seconds and heat the slide on hot plate @ 95 degree Celsius for 1 minute. After that, the sample is exposed under UV light through a chrome mask for 1 minute and developed in AZ 300 MIF Developer for 1 minute. The sample is then rinsed in water for few seconds and dried using argon gas. At this stage, the pattern appears in Photoresist and the underlying PAA as the developer contains water. Next, a mixture of Gox, HSA, and glutaraldehyde is deposited onto patterned PAA and spun @2500 rpm for 30 seconds. The sample is then kept in the incubator (35 Degree Celsius) for 10 minutes to allow the hydrogel to completely form. Next, the sample is submerged into DI water for 5 minutes until the PAA layer dissolves and removes the hydrogel above it, only leaving the hydrogel in desired locations on the device.

This example showed that a sacrificial layer can be sued to pattern hydrogels at a size scale used in the current disclosure. The scheme can be extended to covering devices at both packaged and wafer level.

### Example 2—Photo Patternable Hydrogel

In this example, a photo definable hydrogel was formed by using Poly (ethylene glycol) diacrylate (PEG-DA) mixed with a Photo-initiator (e.g., 2-hydroxy-2methyl-propiophenone). The GOX and HSA solution in DPBS was mixed with a solution of PEG-DA and photo-initiator. The solution used PEG-DA 1 ml mixed with 20 uL of Photo-initiator and 1 mL of the Gox:HSA mixture (1:1) in DPBS. The solution was vortexed and allowed to sit for 5 to 10 minutes before depositing 2 uL of it on a test device @ 2500 rpm. The layer thus formed was exposed by UV light using a chrome mask and was developed in water. The exposed part remained while the unexposed part was washed away. This shows that photo crosslinked hydrogels can be formed at the size scale used in this disclosure. The gel stuck well to the sample and had good uniformity and consistency.

### Example 3—Sensor Manufacture

The sensor circuits were designed in CAD tools using process design kits (e.g., TSMC 180 nm PDK) and were sent to a CMOS foundry (TSMC) for fabrication. After the fabricated sensors were received, they were inspected to match the dimensions and similar physical features with the submitted design. Afterward, they were tested for communication with a corresponding transceiver (also known as a transmitter). Moreover, those were placed on a hot plate and the temperature was varied to see if their data changed to indicate the change in temperature. After this, lithographic postprocessing was started to replace the top metal with more suitable metals. Briefly, AZ5214E photoresist was spun at 4000 rpm, baked at 95 degrees C. for 5 minutes, and exposed using i-Line (e.g., 365 nm UV radiation) exposure in a mask aligner (e.g., MA6) for 5 seconds. Next, a post-exposure bake at 120 degrees C. for 5 minutes was performed followed by a flood exposure for 3 seconds. Next, the resist was developed in the AZ300 developer. This was followed by sputtering of Ti (e.g., 20 nm) followed by Pt (100 nm). After sputtering, a conformal coating was achieved on the sensor electrodes. The next step of post-processing was lift-off to remove metal layers from the unwanted regions by soaking the coated devices in Acetone followed by agitation in an ultrasonic bath. Afterward, another lithography followed by silver deposition, liftoff, and Chlorine exposure through a wet solution (e.g., Ferric Chloride) was performed to create silver-based reference electrodes (e.g., Ag/AgCl). Ag/AgCl RE is more suitable for some applications (e.g., open circuit potential measurements). In a different embodiment, the Pt RE was used as Quasi RE without any Silver deposition. In some embodiments, Pt was deposited in the presence of oxygen plasma to have a thin layer of Pt/PtOx metal on the RE which is a more stable RE material than Pt. Next, the dies were cut using mechanical saw dicing to singulate (separate) the multiple sensing chips. Next, several sensing chips (9 in one example) were die attached to a flexible PCB substrate using a biocompatible (e.g., 31CL) epoxy, followed by baking at 100 C for 1 hour to cure the epoxy. Next, the sensor pads were wire-bonded to corresponding pads on the flexible PCB using 1 mil gold wire in a K&S wire bonder. The wire bonds were then encapsulated in an insulating material (e.g., 31CL) which was then cured at 100 C for 1 hour. Afterward, a connector was soldered to the other end of the flexible PCB to form an interface with the transmitter.

### Example 4—3× Hydrogen Peroxide Sensing

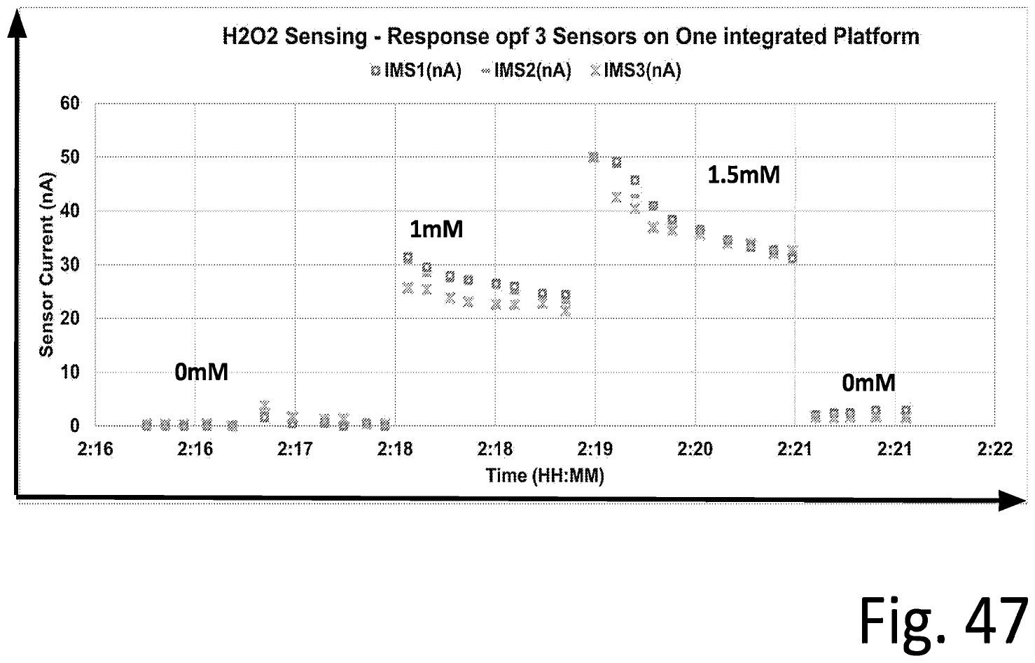

As an example, a sensing chip was fabricated on a CMOS substrate using a 3-electrode design as mentioned in the paragraph above. The sensors were tested in different concentrations of hydrogen peroxide as it is the most common analyte generated by the oxidase-based enzymes in the presence of their substrate e.g., Glucose Oxidase in the presence of glucose, Lactate Oxidase in the presence of Lactate. The sensing chip was connected to the transmitter via the flexible PCB for testing the underlying solid-state sensing platform. The sensor was powered up by the transmitter and the initial current was allowed to stabilize. Once a stable baseline was achieved, the test solution was spiked with small volumes of a stock H₂O₂ solution to create an increasing peroxide concentration in the solution. The testing results as shown in figure ABC indicate that the sensor current increased when peroxide concentration was increased in a statistically significant manner. Similar results were obtained from several sensing chips to establish the validity of the sensor. All 3 on-chip sensors could measure hydrogen peroxide in a similar manner, with small changes due to changes in manual postprocessing which can be eliminated by using automation as utilized in the CMOS process. The average of the sensors as well as the median can be used to reduce noise effects to generate a more accurate signal. The graphs shown in FIG. 47 show these results for one example.

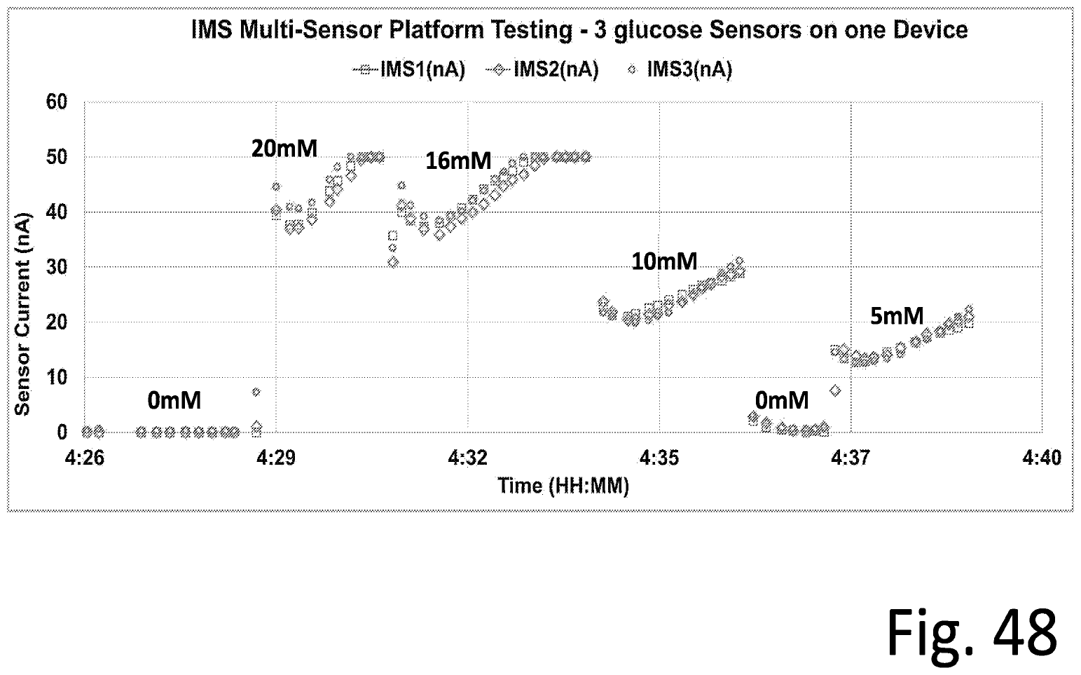

The results in FIG. 47 show that the sensor is quite sensitive to hydrogen peroxide and generates a significantly high current (in 10s of nano amperes) proportional to peroxide concentration. It shows that for the integrated sensor having 3 district sensors on-chip, all 3 are sensitive to peroxide with small variation among them due to process variations (e.g., surface cleaning after packaging). These variations can be substantially decreased by increasing process control, and by using trimming and adding algorithmic features (e.g., calibrating the individual sensors to different slope factors to minimize the differences in current readings). This scheme allows for redundant sensing of and calibration for the same analyte (e.g., glucose), and sensing of different analytes (e.g., glucose, lactate, ketones, creatinine, urea, etc.) on the same chip. An example of the 3 peroxide sensors all coated with the same chemistry (e.g., GOx) is shown in FIG. 48. It shows that all 3 sensors can measure glucose concentration with very small difference among the sensors. The testing was done using small droplets and the droplet drying changes the signal during testing of the same solution. This effect can be minimized by testing in a flow chamber.

### Example 5—Glucose-Hydrogen Peroxide

Glucose is an important analyte for sensing body function. Hydrogen Peroxide can be an indicator of oxidative reactions near a foreign body like the sensing platform. Sensing both together can help the device monitor the extent of oxidative species at the sensor site as well as the glucose concentration.

For glucose sensing, the enzyme (Glucose Oxidase or GOx) was immobilized on the sensor in a hydrogel (e.g., a cross-linked protein matrix) at a thickness of 3 μm. This was done through the immobilization of an enzyme GOx in a hydrogel created by Human Serum Albumin (HSA) with glutaraldehyde as the crosslinking agent. The solutions were made by mixing GOx and HSA (1200 mg, and 1000 mg respectively) in 15 ml DPBS and a crosslinking agent solution of 1% w/w glutaraldehyde (GAD) in DPBS. The GOx-HSA solution and the GAD solution were mixed in a 2:1 ratio by volume. A small volume (e.g., 0.1 microliters) solution from the mixture was dispensed on the sensing chip, followed by spinning at 1000 rpm to more precisely control the thickness of the hydrogel layer. The local deposition of a small volume allowed keeping the enzyme away from the sensor meant to sense Hydrogen Peroxide only. This step was repeated with just an HSA hydrogel (no GOx) to cover the peroxide sensor. Next, a polymer layer (2.5% Polyurethane in THF prepared with HMDI, Jeffamine ED-600, DMS-A15 Mn 3000, DEG, Dibutyltin bis(2-ethylhexanoate) prepared as described in U.S. Pat. No. 5,882,494 herein incorporated by reference in its entirety) was deposited on the enzyme layer and spun at 500 rpm. It serves as a way to control glucose and oxygen diffusion to optimize the sensor response. The sensors were then allowed to stabilize in PBS (Sigma Aldrich, St. Louis MO) for 72 hours and characterized for analyte response. The sensing platform was connected to the transmitter and the assembly was tested in a saline solution. The multi-analyte data was read via the IMS transmitter and the IMS reader application on a PC.

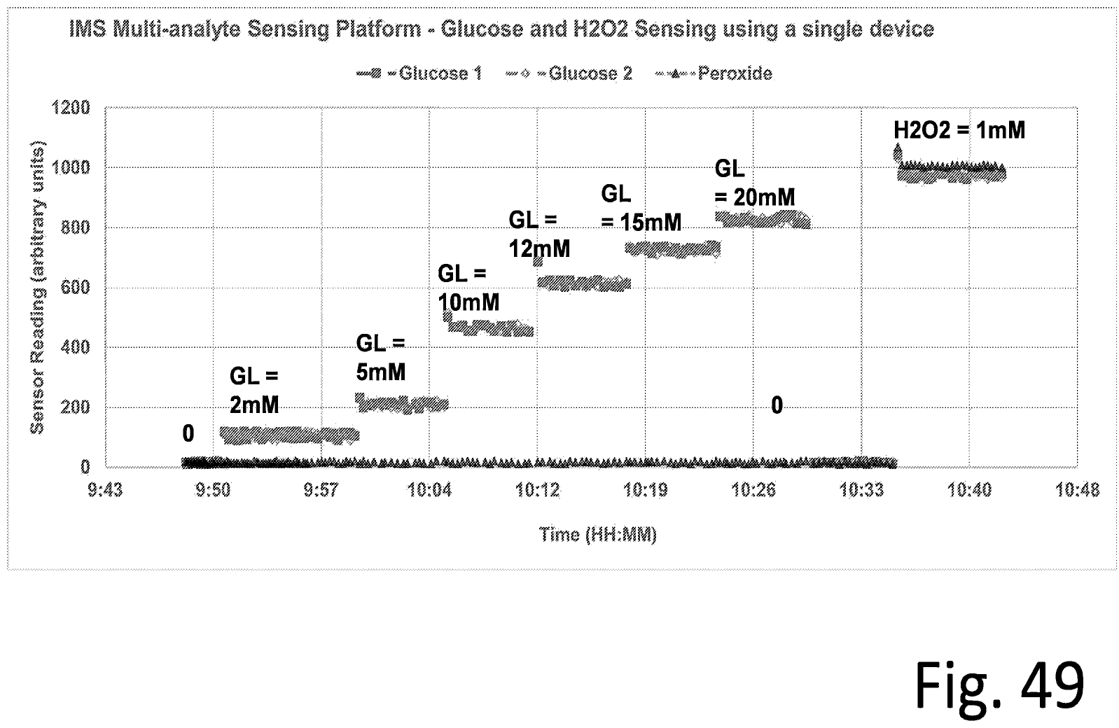

An example of results from a device measuring glucose on two sensors (glucose 1 and glucose 2) and hydrogen peroxide on a third sensor (peroxide) is shown in FIG. 49. One method to pattern the enzyme layer is using a sacrificial layer (e.g., PAA). Another method is using doppler dispensing. Droplet dispensing was used in this example to selectively cover sensors 1 and 2 with enzyme without covering sensor 3. The glucose sensors respond to a change I glucose concentration in the test solution whereas all three sensors respond to a change in Peroxide concentration in the test solution as the underlying sensors are sensitive to hydrogen peroxide. A mathematical analysis of the sensors and the overlapping signal can be done to determine the concentration of both glucose and hydrogen peroxide in a test solution. This combinatorial method can be extended in the case of devices having multiple sensors with multiple coatings to detect multiple analytes. This is known as multivariate calibration to those skilled in the art and explained further in Example 9.

### Example 6—Glucose-Ketones

After Post-processing is done as described in example 1, different enzymes (e.g., GOx and KDH) were immobilized on the sensor in a hydrogel (e.g., a cross-linked protein matrix) at a thickness of 3 μm. This was done through immobilization of the enzyme GOx (Glucose Oxidase) and Ketone (3-hydroxybutyrate) Dehydrogenase (KDH) in a hydrogel created by Human Serum Albumin (HSA) with glutaraldehyde (GAD) as the crosslinking agent. The solutions were made by mixing GOx/KDH and HSA (120 mg, and 100 mg respectively) in 1.5 ml DPBS and a crosslinking agent solution of 1% w/w glutaraldehyde in DPBS. The glucose sensor was made as in the previous example (example 3). For the ketone sensor, a Nicotinamide adenine dinucleotide (NAD+) coenzyme was also added to the solution in an 8:1 (w/w) ratio to the KDH enzyme. The solution was allowed to crosslink after mixing. Once suitable viscosity was achieved (e.g., after 30 minutes of mixing), 0.1 microliters of respective enzyme solutions (e.g., GOx solution, KDH solution) was dispensed on the respective WE on the sensor. Next, a polymer layer (2.5% Polyurethane in THF prepared with HMDI, Jeffamine ED-600, DMS-A15 Mn 3000, DEG, Dibutyltin bis(2-ethylhexanoate) prepared as described in U.S. Pat. No. 5,882,494 herein incorporated by reference in its entirety) was deposited on the enzyme layer and spun at 500 rpm. It serves to control glucose, and oxygen diffusion to optimize the sensor response. The sensors were then allowed to stabilize in PBS (Sigma Aldrich, St. Louis MO) for 72 hours. Next, the flexible PCB panel was laser cut to separate individual sensors for the next steps.

The sensing platform was connected to the transmitter and the assembly was tested in a saline solution. The multi-analyte data was read via the IMS transmitter and the IMS reader application on a PC.

During testing, the sensor was powered up and the initial current was allowed to stabilize. Once a stable baseline was achieved, the test solution was spiked with small volumes of a stock analyte (e.g., glucose/ketone) solution to create an increasing glucose concentration in the solution. The reference concentration was measured using a contour SMBG meter for glucose and with Freestyle Optium Neo for Ketones. As the solution was spiked with more and more analytes (e.g., glucose/ketone), the analyte concentration in the test solution was increased sequentially. Similar results were obtained from several sensors to establish the validity of the sensor.

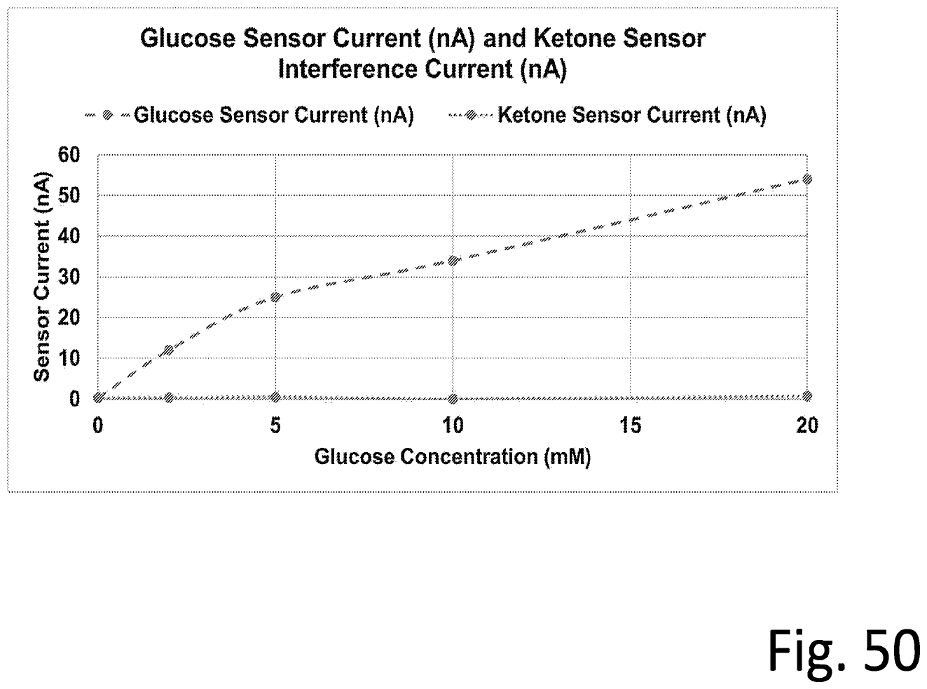

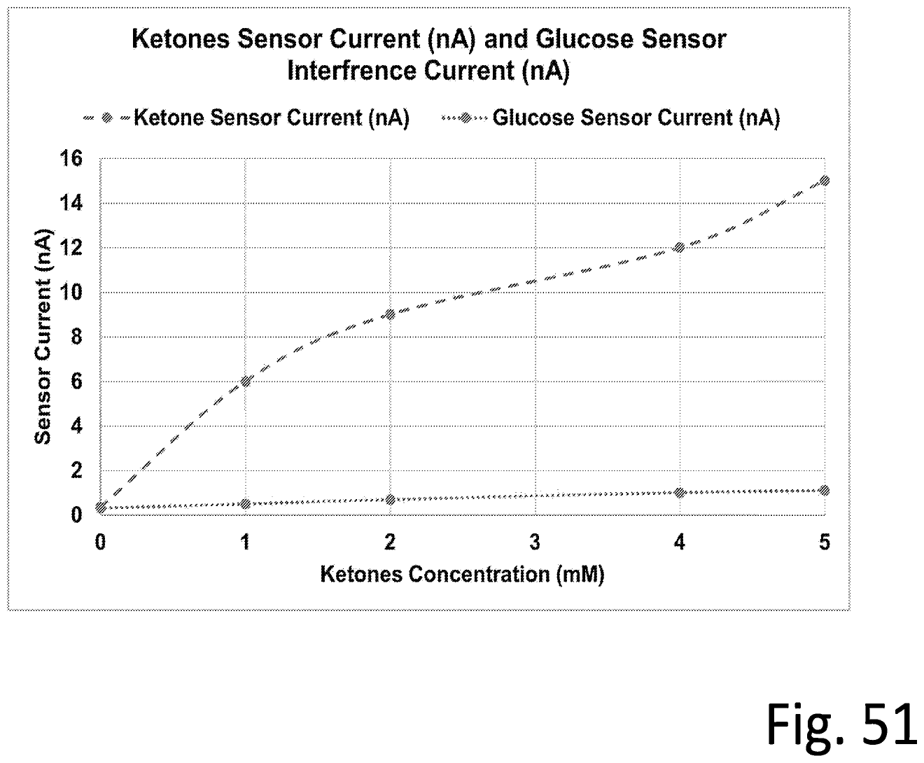

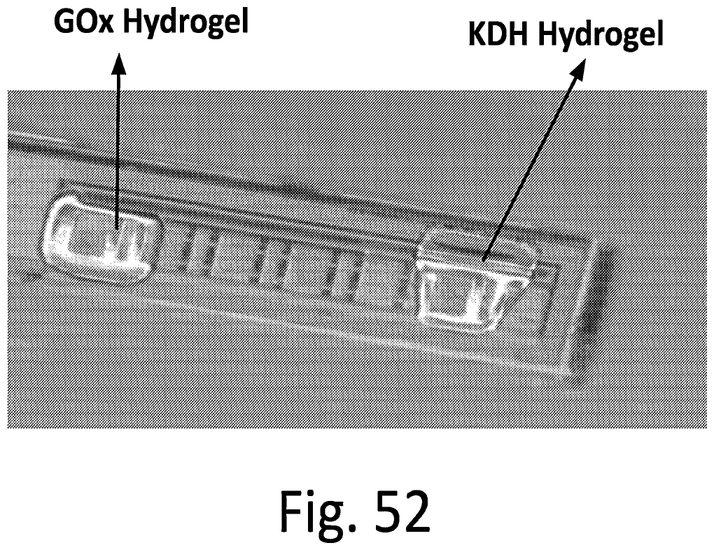

FIG. 50 shows the response of a multi-analyte sensor in which one sensing element is functionalized with GOx (to detect Glucose) and another with KDH (to detect Ketone). The selective coating was done by pipetting the enzyme solutions on the respective electrodes as an example. Nano/pico droplet dispensing can provide finer control on this deposition compared to pipetting. Nevertheless, it shows the platform can measure both glucose and ketone on their respective WE without one interfering with the other substantially. Hence, this shows the platform's ability to detect two analytes at the same time in the same solution using two different WE coated with their respective enzymes. The data from both analyte sensors is multiplexed by the on-chip circuit and is communicated over the single communication channel to the transmitter where it is read, decoded, stored, and analyzed. The graph in FIG. 50 shows that the changes in glucose concentration are detected by the glucose sensor signal and has negligible effect on the ketone sensor and the graph in FIG. 51 shows that changes in Ketones concentration are detected by the Ketones sensor and has a very small effect on the glucose sensor. In this example, both sensors were in a single well. The interference can be further reduced by designing the CMOS ASIC such that both sensors are in a different reaction well defined by top metal, top insulation, and additional insulating materials if desired. This is achieved by using a deeper well design and by increasing the separation between the sensors as well as by using finer chemistry deposition methods which avoid interference among the sensors even more. Nonetheless, the SNR is still acceptable in this application even without these modifications. FIG. 52 shows an example of a CMOS device coated with both GOx and KDH chemistries.

### Example 7—Glucose-Lactate

For glucose sensing, the enzyme (Glucose Oxidase or GOx) was immobilized on the sensor in a hydrogel (e.g., a cross-linked protein matrix) as described in example 2. For lactate sensing, the enzyme lactate oxidase (LOx) was immobilized on a different electrode on the sensing platform. This was done through the immobilization of an enzyme LOx in a hydrogel created by Human Serum Albumin (HSA) with glutaraldehyde as the crosslinking agent. The materials were obtained from Sigma-Aldrich (e.g., LOx: L9795, Bovine Serum Albumin or BSA: A9418, Glutaraldehyde or GAD) for the surface chemistry G7776, PU: 81367, Tetrahydrofuran or THF: 439215). Then, a mixture of LOx and BSA powders was made (50% weight/weight) and was suspended in a Phosphate Buffer Saline (PBS) solution (10% weight/volume). Finally, the GAD solution is mixed with the LOx:BSA solution to result in a final GAD concentration of 0.1% in the mixture. The solution was to let sit for 1 hour to crosslink in situ. Next, a small volume (e.g., 0.1 ul) of the resulting solution was pipetted on the sensor followed by spinning at 1000 rpm to form the enzyme hydrogel layer. Next, a polymer layer (2.5% Polyurethane in THF prepared with HMDI, Jeffamine ED-600, DMS-A15 Mn 3000, DEG, Dibutyltin bis(2-ethylhexanoate) prepared as described in U.S. Pat. No. 5,882,494 herein incorporated by reference in its entirety) was deposited on the enzyme layer and spun at 500 rpm. The sensors were then allowed to stabilize in PBS (Sigma Aldrich, St. Louis MO) for 72 hours.

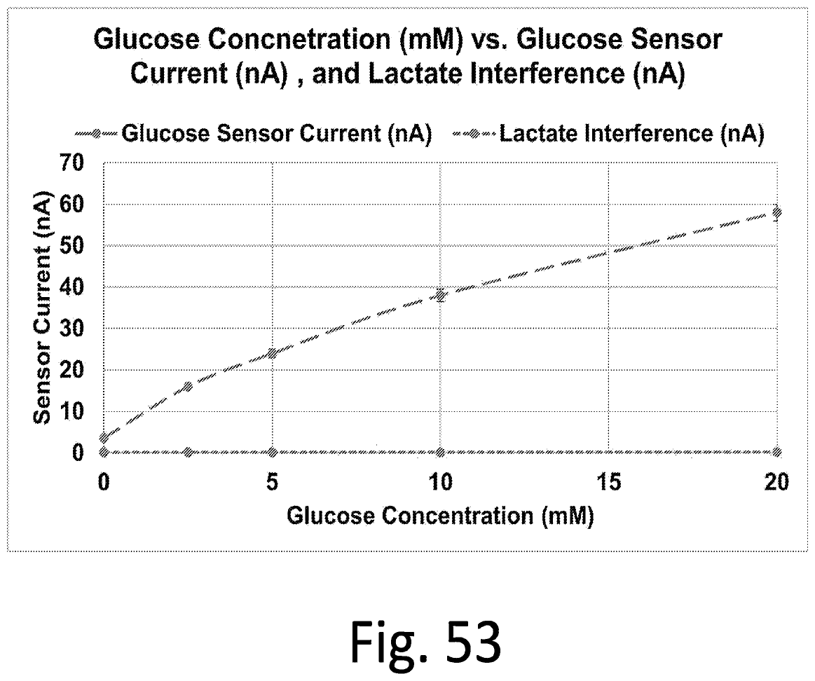

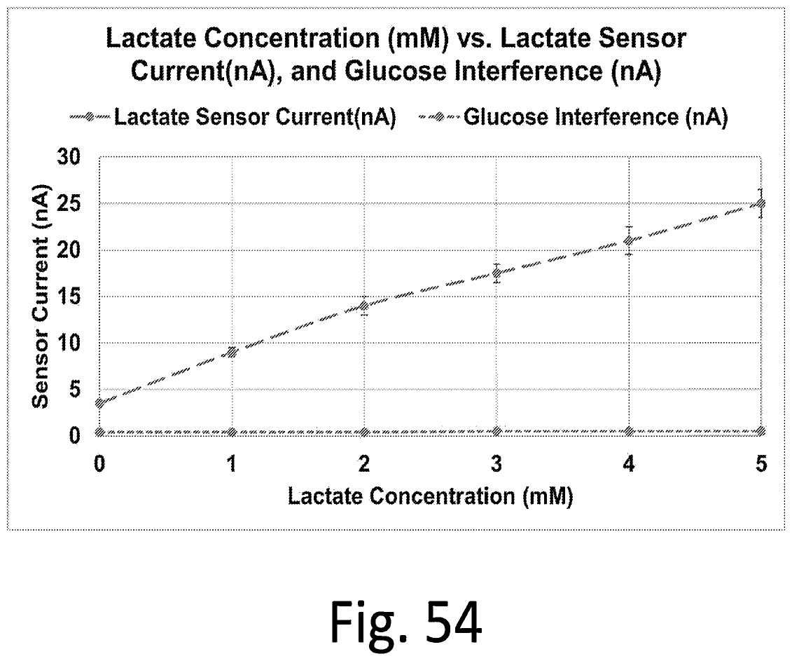

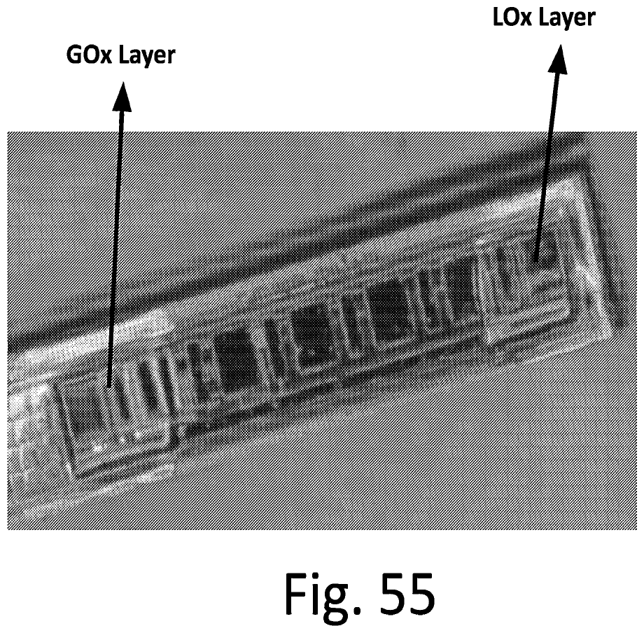

FIG. 53 shows the response of the glucose-lactate multi-analyte sensor to increasing glucose concentrations without any lactate in the solution, followed by FIG. 54 showing the sensor response to increasing lactate concentration without any glucose in the solution. This example shows that the sensors can independently and selectively sense the target analyte without any interference from the other analytes. FIG. 55 shows an example of a CMOS device coated with both GOx and Lox chemistries.

### Example 8—Glucose-Creatinine

The glucose-sensing electrodes were functionalized as in the previous examples. The creatinine-sensing electrodes were functionalized with a solution of creatinase and sarcosine oxidase and HSA (20 mg, 60 mg, 80 mg respectively) with 1% w/w glutaraldehyde in 0.6 mL DPBS. The solution was to let sit for 15 minutes to crosslink in situ. Next, a small volume (e.g., 0.1 ul) of the resulting solution was pipetted on the sensor followed by spinning at 1000 rpm to form the enzyme hydrogel layer. Next, a polymer layer (2.5% Polyurethane in THF prepared with HMDI, Jeffamine ED-600, DMS-A15 Mn 3000, DEG, Dibutyltin bis(2-ethylhexanoate) prepared as described in U.S. Pat. No. 5,882,494 herein incorporated by reference in its entirety) was deposited on the enzyme layer and spun at 500 rpm. The sensors were then allowed to stabilize in PBS (Sigma Aldrich, St. Louis MO) for 72 hours. The sensor was tested by spiking PBS test solution with different concentrations of creatinine. Similar to other examples, the sensor showed successful sensing of glucose and creatinine via the respective sensing electrodes without any significant interference.

### Example 9—Glucose-Hydrogen Peroxide in Humans

The sensors were prepared as in example 2. Then, the sensors were sterilized (e.g., by Synergy Health (San Diego, CA)) using electron-beam sterilization using 25 kGray of e-beam irradiation. Afterward, the sensor was connected to a clean transmitter in a flow hood. The sensor is placed in the upper arm using the applicator (aka the injector). After the sensor is placed in the skin, its data is read via the IMS transmitter and the IMS reader application on a PC.

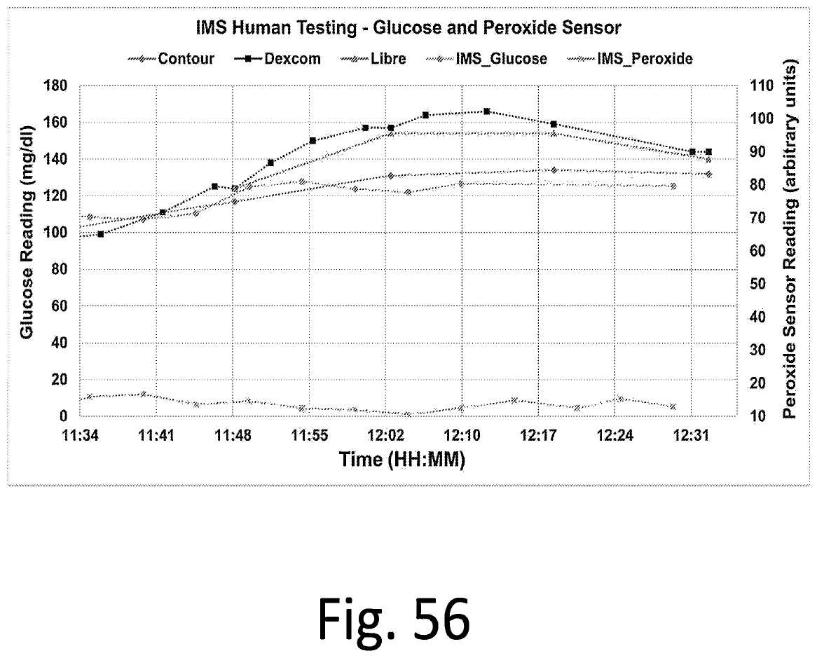

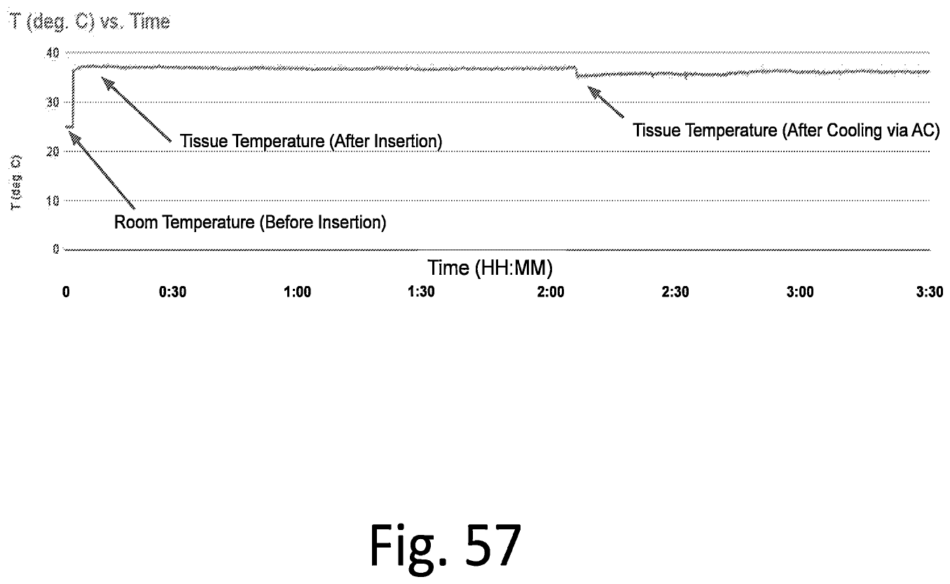

As seen by the data in FIG. 56, the glucose sensor tracked glucose changes in the body. The peroxide sensor was reading the background peroxide level which was low and stable and hence this sensor was reading low. This indicates that the sensor doesn't cause oxidative stress in the tissue. This example validates the multi-analyte sensing capability of the platform in humans. In addition, these experiments also validated tissue temperature monitoring in addition to the chemical analyte monitoring using the integrated sensing device presented here. An example of temperature monitoring in the human using the presented device is shown in FIG. 57. The data shows the device can detect sensor insertion instantaneously due to sudden change in temperature. Moreover, it can also detect change in tissue temperature due to change in environmental conditions (e.g., turning on an Air conditioner). The temperature sensor can also instantaneously detect sensor removal from the tissue (not shown here) due to a sudden change in temperature.

Embodiments of the various aspects described herein can be illustrated by the following numbered paragraphs.
1. A monolithically integrated sensor chip configured for insertion into a patient comprising:
   at least two working electrodes for sensing two or more analytes and generating signals representative of analyte concentration, each working electrode paired with different respective potentiostats at independent working potentials, and
   a communication and power unit connected to the potentiostat, which power and transmits data representative of said analyte concentrations.
2. The monolithically integrated sensor chip of paragraph 1, wherein the independent working potentials are different working potentials.
3. The monolithically integrated sensor chip of any proceeding paragraph, wherein a potentiostat is connected within half a millimeter to the entirety of at least one working electrode.
4. The monolithically integrated sensor chip of any proceeding paragraph, further comprising a temperature sensor.
5. The monolithically integrated sensor chip of any proceeding paragraph, further comprising an digital to analog converter configured to set the independent working potentials of each respective at least two working electrodes.
6. The monolithically integrated sensor chip of any proceeding paragraph, wherein at least one working electrode is coated with a chemistry sensitive to one or a multitude of ketone bodies.
7. The monolithically integrated sensor chip of any proceeding paragraph, wherein at least one working electrode is coated with a chemistry sensitive to lactate.
8. The monolithically integrated sensor chip of any proceeding paragraph, wherein at least one working electrode is coated with a chemistry sensitive to glucose.
9. The monolithically integrated sensor chip of any proceeding paragraph, further comprising an analog to digital converter, wherein the analog to digital converter is electrically connected to the potentiostat.
10. The monolithically integrated sensor chip of any proceeding paragraph, wherein the chip is from 30 microns to 600 microns in thickness, 500 microns to 10,000 microns in length, and in a range from 100 microns to 4,000 microns in width.
11. The monolithically integrated sensor chip of any proceeding paragraph, wherein at least one working electrode is patterned to increase surface area.
12. The monolithically integrated sensor chip of any proceeding paragraph, wherein the at least one working electrode is patterned by forming holes.
13. The monolithically integrated sensor chip of any proceeding paragraph, wherein all potentiostats are configured to be continuously powered by a battery.
14. The monolithically integrated sensor chip of paragraph 1-12, wherein the communication or power unit is configured to operate wirelessly.
15. The monolithically integrated sensor chip of paragraph 1-12, wherein the communication or power unit is configured to operate via a wired connection to a transmitter.
16. The monolithically integrated sensor chip of paragraph 9, furthering comprising a multiplexer electrically connected between the potentiostats associated with at least two working electrodes and the analog to digital converter.
17. The monolithically integrated sensor chip of paragraph 4, further comprising a control logic programmed to process information from the at least two working electrodes by taking into account information from the temperature sensor.
18. An analyte monitoring system comprising:
   the monolithically integrated sensor chip of paragraph 1-13, and 15-17;
   a transcutaneous connector comprising at least one conductive path; and
   a transmitter for containing a battery, the transmitter for placement on top of patient skin.
19. The analyte monitoring system of paragraph 18, wherein the potentiostat is placed at a depth of 0.3 to 10 mm beneath the patient skin.
20. The analyte monitoring system of paragraph 18 or 19, wherein the analyte monitoring system is a continuous analyte monitoring system.
21. A monolithically integrated sensor chip configured for insertion into a patient comprising:
   an integrated sensing and data acquisition unit for sensing two or more analytes and generating signals representative of analyte concentration, said integrated sensing and data acquisition unit having at least two working electrodes, a first working electrode configured to work via a potentiostat and a second working electrode configured to work via voltammetry; and
   an integrated communication and power unit connected to the integrated sensing unit, which powers the integrated sensing and data acquisition unit and transmits data representative of said analyte concentrations.
22. The monolithically integrated sensor chip of paragraph 21, wherein the independent working potentials are different working potentials.
23. The monolithically integrated sensor chip of paragraph 21-22, wherein the potentiostat is connected within half a millimeter to the entirety of the first working electrode.
24. The monolithically integrated sensor chip of paragraph 21-23, further comprising a temperature sensor.
25 The monolithically integrated sensor chip of paragraph 21-24, further comprising an digital to analog converter configured to set the independent working potentials of each respective at least two working electrodes.
26. The monolithically integrated sensor chip of paragraph 21-25, wherein at least one working electrode is coated with a chemistry sensitive to one or more ketone bodies.
27. The monolithically integrated sensor chip of paragraph 21-26, wherein at least one working electrode is coated with a chemistry sensitive to lactate.
28. The monolithically integrated sensor chip of paragraph 21-27, wherein at least one working electrode is coated with a chemistry sensitive to glucose.
29. The monolithically integrated sensor chip of paragraph 21-28, further comprising an analog to digital converter, wherein the analog to digital converter is electrically connected to the potentiostat.
30. The monolithically integrated sensor chip of paragraph 21-29, wherein the chip is from 30 microns to 600 microns in thickness, 500 microns to 10,000 microns in length, and in a range from 100 microns to 4,000 microns in width.
31. The monolithically integrated sensor chip of paragraph 21-30, wherein at least one working electrode is patterned to increase surface area.
32. The monolithically integrated sensor chip of paragraph 21-31, wherein at least one working electrode is patterned by forming holes.
33. The monolithically integrated sensor chip of paragraph 21-32, wherein all potentiostats are configured to be continuously powered by a battery.
34. The monolithically integrated sensor chip of paragraph 21-33, wherein the communication and power unit are configured to operate wirelessly.
35. The monolithically integrated sensor chip of paragraph 21-32, wherein the communication and powered unit is configured to operate via a wired connection to a transmitter.
36. The monolithically integrated sensor chip of paragraph 29, furthering comprising a multiplexer electrically connected between the potentiostats associated with at least two working electrodes and the analog to digital converter.
37. The monolithically integrated sensor chip of paragraph 24, further comprising a control logic programmed to process information from the at least two working electrodes by taking into account information from the temperature sensor.
38. A continuous sensor assembly comprising a flexible substrate and a monolithically integrated sensor chip comprising at least two working electrodes and surrounded on all sides by the flexible substrate except for wherein a top portion of the monolithically sensor chip is open to an analyte.
39. The continuous sensor assembly of paragraph 38, wherein the monolithically integrated sensor chip is of paragraph 1-20 or 21-37.
40. The continuous sensor assembly of paragraph 38 or 39, wherein the flexible substrate has a rigidity enforcing member.
41. The continuous sensor assembly of paragraph 40, wherein the rigidity enforcing member is a metal and has a thickness of 5 to 60 microns.
42. The continuous sensor assembly of paragraph 41, wherein the thickness of the rigidity enforcing members is about 30 microns.
43. A method of packaging a monolithically integrated sensor chip, said method comprising:
   bonding a front of the monolithically integrated sensor chip to a conductor located on a flexible substrate, and
   covering the entirety of a back of the monolithically integrated sensor chip with a flexible material,
   wherein the flexible substrate and the flexible material cover all of the sides and back of the monolithically integrated sensor chip and only a portion of the front of the monolithically integrated sensor chip.
44. The method of paragraph 43, wherein the monolithically integrated sensor chip is of paragraph 1-20 or 21-37
45. A method of post-processing a monolithically integrated sensor chip, said method comprising lithographic patterning, metal etching, physical vapor deposition, and dicing such that parts of the CMOS sensor chip are coated with two different suitable material including two different noble metals while the remaining of the integrated sensor chip is not coated with either material.
46. The method of paragraph 45, wherein the post-processing is done at wafer scale.
47. A method of functionalizing a monolithically integrated sensor chip comprising at least two noble metal coated working electrodes, said method comprising:
   A) coating a portion of the monolithically integrated sensor chip with a water-dissolvable sacrificial layer;
   B) coating a portion of the monolithically integrated sensor chip with a mask;
   C) patterning the mask to open the mask over a first noble metal coated working electrode;
   D) transfer to the sacrificial layer by exposure to water creating an opening over the first noble metal coated working electrode;
   E) coating the first working electrode(s) with a first hydrogel; and
   F) coating the wafer in an excess of water.
48. The method of paragraph 47, further comprising:
   G) coating a portion of the monolithically integrated sensor chip with a water-dissolvable sacrificial layer;
   H) coating a portion of the monolithically integrated sensor chip with a mask;
   I) patterning the mask to open the mask over a second noble metal coated working electrode;
   J) transfer to the sacrificial layer by exposure to water creating an opening over the second noble metal coated working electrode;
   K) coating the second working electrode(s) with a second hydrogel different from the first hydrogel; and
   L) coating the wafer in an excess of water to form a monolithically integrated sensor chip with two working electrodes with two different coated hydrogels.
49. The method of paragraph 47 or 48, wherein the sacrificial layer is water soluble polyacrylic acid (PAA).
50. The method of paragraph 47-49, wherein the mask is a photo patternable material.
51. A method of attaching a monolithically integrated sensor configured to measure at least two analytes to a flexible electronics connector for placement within a human subject, said method comprising:
   attaching the monolithically integrated sensor configured to measure at least two analytes to the electronic connector by placing a conductor between a contact pad of the monolithically integrated sensor and a contact pad of the electronics connector,
   wherein the overall thickness of the integrated sensor and flexible electronics connector after attachment is less than 1 mm and protects the contact pads to avoid contact with human fluids but provides for access of human fluids to integrated sensing elements of the integrated sensor permitting measurement of the at least two analytes.
52. The method of paragraph 51, wherein the overall thickness of the integrated sensor and flexible electronics connector after attachment is less than 150 microns.
53. A method of adding at least two functional chemistry layers sensitive to at least 2 different analytes on a monolithically integrated sensor on a CMOS wafer to selectively coat the sensing electrodes, said method comprising:
   coating a first functional chemistry layer on sensing electrode(s) sensitive to a first analyte on the CMOS wafter by pico droplet deposition, spin coating, lithographic patterning, spray coating, stencil coating, or dip coating, and
   coating a second functional chemistry layer on sensing electrode(s) sensitive to a second analyte on the CMOS wafer by pico droplet deposition, spin coating, lithographic patterning, spray coating, stencil coating, or dip coating.
54. The method of paragraph 53, wherein the first functional chemistry layer is coated at a thickness of less than 100 μm.
55. The method of paragraph 53, wherein the first functional chemistry layer is coated at a thickness of less than 10 μm.
56. A transmitter system for placement on patient skin comprising an integrated circuit and a power source both located on a flexible electronics substrate and used to power and communicate with a subdermal sensor configured to measure one or more analytes, wherein the transmitter is smaller than 3 cm in the largest dimension and weighs less than 20 grams.
57. The method of paragraph 56, wherein the transmitter is encased in a thin housing with a thickness of less than 5 mm that protects it from environment without the use of hard casing.
58. A transmitter system for placement on patient skin comprising an integrated circuit and a power source both located on a rigid-flexible printed circuit board including a rigid substrate and a flexible substrate, said integrated circuit and power source used to power and communicate with a subdermal sensor configured to measure one or more analytes, wherein the rigid substrate is smaller than 3 cm in the largest dimension and weighs less than 20 grams, wherein the sensor is connected to the flexible substrate, and wherein the rigid and the flexible substrates are connected with conductive vias without the use of an interfacial connector.
59. The method of paragraph 58, wherein the transmitter is encased in a thin housing with a thickness of less than 5 mm that protects it from environment without the use of hard casing.

While specific embodiments of the invention have been described above, it will be appreciated that the invention may be practiced otherwise than as described and that that the described embodiments are for all purposes exemplary, not limiting. Various modifications can be made to the described embodiments without departing from the scope of the present invention which is defined by the appended claims.

1.-59. (canceled)

60. A monolithically integrated sensor chip configured for insertion into a patient comprising: at least two working electrodes for sensing two or more analytes and generating signals representative of analyte concentration, each working electrode paired with different respective potentiostats at independent working potentials, and a communication and power unit connected to the potentiostat, which powers and transmits data representative of said analyte concentrations.

61. The monolithically integrated sensor chip of claim 60, wherein the independent working potentials are different working potentials.

62. The monolithically integrated sensor chip of claim 60, wherein a potentiostat is connected within half a millimeter to the entirety of at least one working electrode.

63. A monolithically integrated sensor chip configured for insertion into a patient comprising: an integrated sensing and data acquisition unit for sensing two or more analytes and generating signals representative of analyte concentration, said integrated sensing and data acquisition unit having at least two working electrodes, a first working electrode configured to work via a potentiostat and a second working electrode configured to work via voltammetry; and an integrated communication and power unit connected to the integrated sensing unit, which powers the integrated sensing and data acquisition unit and transmits data representative of said analyte concentrations.

64. The monolithically integrated sensor chip of claim 63, wherein the potentiostat is connected within half a millimeter to the entirety of the first working electrode.
